(19) United States
(12) Patent Application Publication
(10) Pub. No.: US 2009/0012612 A1
(43) Pub. Date: Jan. 8, 2009

(54) DEVICES AND METHODS FOR PUSH-DELIVERY OF IMPLANTS

(76) Inventors: David WHITE, Atherton, CA (US); Janine C. Robinson, Half Moon Bay, CA (US); Michael Hogendijk, Mountain View, CA (US)

Correspondence Address: MORRISON & FOERSTER LLP, 755 PAGE MILL RD, PALO ALTO, CA, 94304-1018, US

(21) Appl. No.: 12/212,587

(22) Filed: Sep. 17, 2008

Related U.S. Application Data
(63) Continuation-in-part of application No. 11/862,095, filed on Sep. 26, 2007.
Application No. 11/862,095 is a continuation-in-part of application No. 12/099,296, filed on Apr. 8, 2008.
(60) Provisional application No. 60/911,056, filed on Apr. 10, 2007.
(60) Provisional application No. 60/975,444, filed on Sep. 26, 2007.

(51) Int. Cl. A61F 2/02; A61B 17/58

(52) U.S. Cl. 623/11.11; 606/99

(57) ABSTRACT

Devices and treatments for a joint include a resilient elongate orthopedic device inserted into a joint space using a delivery instrument. The orthopedic device is housed in a delivery cavity of the delivery instrument delivered to an implantation site using a push mechanism. The delivery instrument optionally comprises a guide or tongue structure to facilitate positioning of the delivery instrument about the implantation site.

### CROSS-REFERENCE TO RELATED APPLICATIONS

This application is a) a continuation-in-part of U.S. application Ser. No. 11/862,095, filed Sep. 27, 2007, which claims priority under 35 U.S.C. § 119(e) to U.S. Provisional Ser. No. 60/911,056, filed Apr. 10, 2007, and to U.S. Provisional Ser. No. 60/975,444, filed Sep. 26, 2007, and is also b) a continuation-in-part of U.S. application Ser. No. 12/099,296, filed Apr. 8, 2008, all of the above which are hereby incorporated by reference in their entirety.

### BACKGROUND OF THE INVENTION

Today, there are an increasing number of patients with osteoarthritis, rheumatoid arthritis, and other joint degenerative processes. Osteoarthritis is by far the most common type of arthritis, and the percentage of people who have it grows higher with age. An estimated 12.1 percent of the U.S. population (nearly 21 million Americans) age 25 and older have osteoarthritis of one form or another. Although more common in older people, it usually is the result of a joint injury, a joint malformation, or a genetic defect in joint cartilage. The incidence and prevalence of osteoarthritis differs among various demographic groups: osteoarthritis tends to start for men before the age of 45, and after the age of 45 it is more common in women. It is also more likely to occur in people who are obese or overweight and is related to those jobs that stress particular joints.

Arthritis is a degenerative process that affects the musculoskeletal system and specifically the joints, where two or more bones meet. It most often occurs in the joint of the hands and wrists (particularly in the fingers and thumbs, between the phalanges, the metacarpals and/or the carpals), feet (in the toes, between phalanges, metatarsals and/or tarsals), ankles, elbows, shoulders, knees, hips, and the spine (particularly at the neck and lower back). Joint problems can include inflammation and damage to joint cartilage (the tough, smooth tissue that covers the ends of the bones, enabling them to glide against one another) and surrounding structures. Such damage can lead to joint stiffness, weakness, instability and visible deformities that, depending on the location of joint involvement, can interfere with the basic daily activities such as walking, climbing stairs, using a computer keyboard, cutting food and brushing teeth. This ultimately results in moderate to severe pain. Drug regimes can provide temporary relief from the pain but do not slow down the crippling affects. Drugs may also subject patients to serious side effects and risks, such as the increased cardiovascular risks associated with osteoarthritis drugs Vioxx and Bextra, which were withdrawn from the market. Drugs used to treat other forms of arthritis, such as corticosteroids, are associated with osteoporosis and hyperglycemia and can lead to increased risks of bone fracture and diabetes, for example. When pharmacologic therapy and physical therapy no longer provide adequate relief, only surgical options remain.

The extreme result or end point in traditional treatments is an open surgical procedure to implant a spacer or to perform total joint replacement with a prosthetic device. Current joint replacement therapies (spacers or a total prosthesis) require the joint capsule to be surgically opened and the bone surfaces to be partially or totally removed. Both modalities present various drawbacks. For example, U.S. Pat. No. 6,007,580 to Lehto et al. describes an implantable spacer that must be fixed at one or both ends to the bone of either end of the knuckle (e.g. the metacarpal-phalangeal (MCP) joint). The spacer must be implanted by opening of the joint capsule and must be affixed at one or both ends to the corresponding bone faces.

Various spacers in the art can cause inflammation, while total joint replacement can limit the range of motion and also compromise the strength and stability of the joint. These surgeries are highly invasive and require the joint capsule to be surgically opened, and the incision itself can result in inflammation and infection. Due to the invasiveness of the procedure, prolonged healing times are required. Furthermore, the invasive nature of these surgeries sometimes precludes a second joint replacement or spacer when the first joint device wears out or fails.

It would be desirable as well as beneficial if there were an intermediary step or alternative treatment before subjecting patients to drastic joint replacement and/or long-term drug therapy.

### BRIEF SUMMARY OF THE INVENTION

Various embodiments disclosed herein relate generally to the treatment of osteoarthritis, rheumatoid arthritis, and other degenerative joint processes, and include but are not limited to minimally invasive implantable devices to reduce bone-to-bone contact in a joint.

Systems and methods for treating degenerative joint conditions include an orthopedic device comprising a resilient elongate member, which may be implanted in a joint space using a suture. The suture is passed through the joint space and used to pull the orthopedic device into the joint space. The suture may be inserted using a percutaneously inserted needle or other type of needle-based delivery instrument. The orthopedic device may be restrained to a reduced profile that permits minimally invasive implantation, but changes to an enlarged profile when positioned at an implantation site. The orthopedic device may comprise a shape-memory material, and may comprise an open or closed shape configuration.

In one example, a joint treatment system is provided, including a delivery instrument comprising a housing, a housing cavity, a delivery opening in communication with the housing cavity, a slidable actuator joined to a push member, and a tongue member protruding from the housing in proximity to the delivery opening, wherein the push member has a movement path, and an non-linear orthopedic device located in the housing cavity, the non-linear orthopedic device comprising a first end, a second end, a non-linear body therebetween, wherein the non-linear orthopedic device is oriented in the housing cavity so that the non-linear body is in closer proximity to the delivery opening than both the first and second ends. In some examples, the on-linear orthopedic device is an arcuate orthopedic device. Optional features of the delivery instrument include a mounting member located in the housing cavity between the delivery opening and at least a portion of the slidable actuator, onto which the non-linear orthopedic device may be mounted. The non-linear orthopedic device may be oriented so that a transverse dimension relative to the movement path of the push member is greater than a corresponding transverse dimension of the delivery opening. In certain examples, the push member may be configured to pass through at least a portion of the mounting member and/or configured with a distal end having a complementary shape to a surface of the non-linear orthopedic device. In some further examples, the non-linear orthopedic device may be configured with a first position and a second position in the housing cavity, wherein the second position is closer to the delivery opening. The tongue member may have any of a variety of optional features and configurations, including but not limited to a tapered configuration, and/or a blunt tip section having a maximum transverse dimension that is smaller than the larger transverse dimension of the orthopedic device. In some embodiments, the delivery instrument comprises at least two tongue members, and in further embodiments, at least one of the tongue members may be biased toward another tongue member, and may even be configured such that at least two of the tongue members are biased into contact each other. One or more tongue members may also comprise a cutting structure. The tongue member may also be configured to displace away from the delivery opening by the non-linear orthopedic device.

In another example, a joint treatment system is provided, including a delivery instrument comprising a movable actuating assembly and a movement passage having a cross-sectional area with minimum first dimension and a second dimension transverse to the first dimension, and an non-linear elongate orthopedic device releasably coupled to the delivery instrument, the non-linear elongate orthopedic device having a first end, a second end, and a non-linear body therebetween and a non-linear longitudinal axis between the first and second ends, wherein the non-linear elongate orthopedic device is oriented with respect to the delivery instrument such that a portion of the non-linear longitudinal axis is transverse to the movement passage of the movable actuating assembly. The delivery instrument may further comprise a housing with a housing cavity and a delivery opening in communication with the housing cavity, and/or a tapered delivery opening. Also, the portion of the non-linear longitudinal axis of the orthopedic device that is transverse to the movement passage of the movable actuating assembly may be located between the movable actuating assembly and the delivery opening. In use, the cross-sectional area with the minimum first dimension and the second dimension transverse to the first dimension may be located at the delivery opening. In some examples, the movable actuating assembly may comprise a slidable member and a push member, and the delivery instrument may further comprise a first guide member distal to the cross-sectional area with the minimum first dimension and the second dimension transverse to the first dimension. The first guide member may be a flat and/or may be flexible. In one particular example, flat guide member comprises a tongue member with a tapered configuration and a rounded distal tip. The delivery instrument may also further comprise a mounting member between at least a portion of the movable actuating assembly and the cross-sectional area with the minimum first dimension and the second dimension transverse to the first dimension, and the non-linear elongate orthopedic device may be releasably mounted on the mounting member. In some examples, at least a portion of the movable actuating assembly may be movably positionable through the mounting member. The delivery instrument may also optionally comprise a second guide member about the delivery opening, and in some instances, at least one of the first and second guide members are at least partially biased toward the other guide member, and at least one of the first and second guide members may even be configured to separate from the other guide member with passage of the orthopedic device between the first and second guide members.

In another example, a method for treating a joint is provided, comprising positioning an orthopedic device about a joint, wherein orthopedic device comprises a first end, a second end, and a non-linear body therebetween and a non-linear longitudinal axis between the first and second ends, and pushing against the non-linear body such that at least a portion of the non-linear body enters the joint before the first and seconds ends. The positioning of the orthopedic device about the joint may be using a delivery system containing or holding the orthopedic device. The delivery system may include a guide assembly, which may be inserted into the tissue surrounding the joint, and even at least partially into the joint space. The method may also include increasing access to the joint space along at least one dimension of tissue adjacent to the joint space while inserting the guide assembly. The guide assembly may be configured, such that guide assembly may be bent or flexed when used. In some examples, bending the guide assembly comprises bending a first guide member of the guide assembly, and in examples comprising at least two guide members, bending the first guide member may comprise bending or flexing the first guide member away from a second guide member of the guide assembly, and/or bending or flexing the second guide member from the first guide member. Bending the first guide member may occur while passing or forcing the non-linear body against a surface of the first guide member, which may occur in some examples while pushing the non-linear body. After implantation, the guide member may be withdrawn from the tissue surrounding the joint.

### BRIEF DESCRIPTION OF THE DRAWINGS

These and other features will now be described in connection with various embodiments herein, in reference to the accompanying drawings. The illustrated embodiments, however, are merely examples and are not intended to limit the claimed subject matter.

FIG. 1A is a schematic top view of one embodiment of an orthopedic device comprising a substantially straightened configuration.

FIG. 1B is a schematic top view of one embodiment of an orthopedic device comprising an open hoop arcuate configuration.

FIG. 1C is a schematic top view of one embodiment of an orthopedic device comprising a nautilus-style spiral arcuate configuration.

FIG. 1D is a schematic top view of one embodiment of an orthopedic device comprising a closed polygonal configuration.

FIG. 1E is a schematic top view of the orthopedic device of FIG. 1D in a collapsed delivery configuration.

FIG. 1F is a schematic top view of one embodiment of an orthopedic device comprising a closed circular configuration.

FIG. 1G is a schematic top view of the orthopedic device of FIG. 1F in a collapsed delivery configuration.

FIG. 2 is a schematic cross-sectional view perpendicular to a longitudinal axis of an embodiment of an orthopedic device comprising an elongate core and an articular layer surrounding at least a portion of the core.

FIG. 3A is a schematic cross-sectional view along a plane substantially parallel to and passing through a longitudinal axis of an embodiment of an orthopedic device having a substantially straightened configuration and comprising an elongate core and an articular layer surrounding at least a portion of the core.

FIG. 3B is a schematic cross-sectional view along a plane substantially parallel to and passing through a longitudinal axis of an embodiment of an orthopedic device having an open hoop arcuate configuration, the device comprising an elongate core and an articular layer surrounding at least a portion of the core.

FIG. 3C is a schematic cross-sectional view along a plane substantially parallel to and passing through a longitudinal axis of an embodiment of an orthopedic device having a nautilus-style spiral arcuate configuration, the device comprising an elongate core and an articular layer surrounding at least a portion of the core.

FIG. 3D is a schematic cross-sectional view along a plane substantially parallel to and passing through a longitudinal axis of an embodiment of an orthopedic device having an open hoop arcuate configuration, the device comprising one or more elongate cores wrapped, braided or folded along a length of the device and an articular layer surrounding at least a portion of the core.

FIG. 3E is a schematic cross-sectional view along a plane substantially parallel to and passing through a longitudinal axis of an embodiment of an orthopedic device having a nautilus-style spiral arcuate configuration, the device comprising one or more elongate cores wrapped, braided or folded along a length of the device and an articular layer surrounding at least a portion of the core.

FIG. 3F is a schematic planar cross-sectional view of the orthopedic device of FIG. 1F.

FIG. 3G is a schematic planar cross-sectional view of the orthopedic device of FIG. 1G.

FIG. 4A is a schematic side view of an embodiment of an elongate core comprising one or more substantially linear or straight members.

FIG. 4B is a schematic side view of an embodiment of an elongate core comprising one or more wave, curve or zig-zag members disposed in one or more planes.

FIG. 4C is a schematic side view of an embodiment of an elongate core comprising one or more members in a braided or weave configuration.

FIG. 5A is a schematic top view of an embodiment of an elongate core comprising an open hoop arcuate configuration and one or more end pieces.

FIG. 5B is a schematic top view of an embodiment of an elongate core comprising an open hoop arcuate configuration and one or more bends or hooks.

FIG. 5C is a schematic top view of an embodiment of an elongate core comprising an open hoop arcuate configuration and one or more features bent in or out of the primary plane of the device.

FIG. 5D is a schematic side view of an embodiment of an orthopedic device comprising a multi-planar spiral configuration.

FIG. 5E is a schematic side view of an embodiment of an orthopedic device comprising a multi-planar arcuate configuration.

FIG. 5F is a schematic side view of an embodiment of an orthopedic device comprising a “W”-shape configuration.

FIGS. 6A to 6K are schematic cross-sectional views of various embodiments of elongate cores.

FIGS. 6L to 6S are schematic superior and cross-sectional views of various embodiments of orthopedic devices with non-circular cross-sectional shapes.

FIGS. 6T to 6W are schematic superior and cross-sectional views of various embodiments of an orthopedic device with a membrane member.

FIG. 6X is a schematic superior view of additional embodiment of orthopedic device with membrane member.

FIG. 6Y is a schematic superior view of an embodiment of an orthopedic device comprising a textured surface.

FIG. 6Z is a schematic superior view of an embodiment of an orthopedic device comprising one or more retaining structures.

FIG. 7A is a schematic perspective view of an embodiment of an orthopedic device comprising a plurality of independent or inter-connectable discrete elongate members.

FIG. 7B is a schematic perspective view of an embodiment of an orthopedic device comprising a plurality of independent or inter-connectable discrete elongate members in a “W”-shape configuration.

FIG. 8 is a schematic perspective view of an embodiment of an orthopedic device comprising a plurality of independent or inter-connectable discrete members.

FIG. 9A is a schematic side view of an embodiment of an elongate core comprising a plurality of inter-connectable discrete members in a substantially straightened configuration.

FIG. 9B is a schematic side view of an inter-connectable discrete member of FIG. 9A.

FIG. 9C is a schematic side view of an embodiment of an elongate core comprising a plurality of inter-connectable discrete members according to FIG. 9A in an arcuate open loop configuration.

FIG. 10A is a schematic side view of an orthopedic device delivery system according to one embodiment of the present invention comprising a handle and a plunger.

FIG. 10B is a schematic side view of an orthopedic device delivery system according to one embodiment of the present invention comprising a substantially straight cannula or needle with a lumen.

FIG. 10C is a schematic side view of an orthopedic device delivery system according to one embodiment of the present invention comprising an arcuate cannula or needle with a lumen.

FIG. 10D is a schematic side view close up of a distal end of an orthopedic device delivery system according to one embodiment of the present invention comprising a blunted delivery cannula.

FIG. 10E is a schematic side view of an orthopedic device delivery system according to one embodiment of the present invention comprising an angular tip.

FIG. 11 is a schematic side view of an orthopedic device delivery system according to one embodiment of the present invention comprising an implantable orthopedic device, a cannula, and a plunger.

FIG. 12A is a schematic side cross-sectional view of an orthopedic device delivery system according to one embodiment of the present invention prior to implantation in a joint.

FIG. 12B is a schematic top cross-sectional view orthogonal to FIG. 12A of two embodiments of orthopedic device delivery systems similar to the system of FIG. 12A prior to implantation in a joint, wherein on embodiment comprises a substantially straight cannula and the other embodiment comprises an arcuate cannula.

FIG. 13A is a schematic side cross-sectional view of an orthopedic device delivery system according to the embodiment of the present invention shown in FIG. 12A upon partial insertion of the orthopedic device into the joint.

FIG. 13B is a schematic top cross-sectional view orthogonal to FIG. 13A of two embodiments of orthopedic device delivery systems according to FIG. 12B upon partial insertion of the orthopedic device into the joint.

FIG. 14A is a schematic side cross-sectional view of an orthopedic device delivery system according to the embodiment of the present invention shown in FIG. 12A upon deployment of the orthopedic device into the joint.

FIG. 14B is a schematic top cross-sectional view orthogonal to FIG. 14A of two embodiments of orthopedic device delivery systems according to FIG. 12B upon deployment of the orthopedic device into the joint.

FIG. 15A is a schematic side cross-sectional view of an orthopedic device delivery system according to the embodiment of the present invention shown in FIG. 12A upon deployment of the orthopedic device into the joint and removal of the delivery cannula.

FIG. 15B is a schematic top cross-sectional view orthogonal to FIG. 15A of two embodiments of orthopedic device delivery systems according to FIG. 12B upon deployment of the orthopedic device into the joint and removal of the delivery cannula(s).

FIG. 16A is a schematic side view of an orthopedic device according to one embodiment of the present invention comprising a tether and a loop structure in a substantially straightened configuration.

FIG. 16B is a schematic side view of the orthopedic device of FIG. 16A in an arcuate configuration.

FIG. 16C is a schematic side view of an orthopedic device according to one embodiment of the present invention comprising one or more tethers in an arcuate configuration.

FIG. 17 is a schematic side view of an orthopedic device according to one embodiment of the present invention comprising a looped arcuate configuration and at least one anchor.

FIG. 18 is a schematic side view of an orthopedic device removal system according to one embodiment of the present invention comprising an implantable orthopedic device, a cannula, and a snare.

FIGS. 19A and 19B are schematic perspective and side views of a portion of an interface in an orthopedic device delivery and removal system according to one embodiment of the present invention comprising an implantable orthopedic device and a plunger connectable with a device interface.

FIGS. 20A to 20C are schematic side views of a portion of an interface in an orthopedic device delivery and removal system according to another embodiment of the present invention comprising an implantable orthopedic device and a plunger connectable with a device interface.

FIG. 21A is a schematic perspective view of an orthopedic device delivery system according to one embodiment of the present invention comprising a loading device for storing the orthopedic device in an arcuate configuration.

FIG. 21B is a schematic side view of the orthopedic device delivery system comprising a loading device of FIG. 21A.

FIG. 21C is a schematic side view of an orthopedic device delivery system according to one embodiment of the present invention comprising a loading device comprising a needle and a loop for storing the orthopedic device in an arcuate configuration.

FIG. 22A is a schematic side view of an orthopedic device delivery system according to one embodiment of the present invention comprising a loading device cassette and a cannula or needle with a channel.

FIG. 22B is a schematic side view of the orthopedic device delivery system of FIG. 22A with an orthopedic device being advanced from the loading device cassette and into the lumen of the cannula or needle.

FIG. 23 is a schematic perspective view of an orthopedic device delivery system according to one embodiment of the present invention comprising a cassette and a needle with a lumen.

FIG. 24 is a schematic side view of an orthopedic device delivery system according to one embodiment of the present invention comprising a plunger.

FIG. 25 is a schematic perspective view of an orthopedic device delivery system according to one embodiment of the present invention comprising a cassette barrel with an orthopedic device groove.

FIG. 26 is a schematic side view of an orthopedic device delivery system according to one embodiment of the present invention comprising a cassette barrel with an orthopedic device groove.

FIG. 27A is a partial cut-away schematic side view of an orthopedic device delivery system according to one embodiment of the present invention comprising a cassette, barrel and plunger.

FIG. 27B is a partial cut-away schematic side view of an orthopedic device delivery system according to one embodiment of the present invention comprising a cassette, barrel and plunger.

FIGS. 28A to 28E are partially exploded cut-away schematic side views of an orthopedic device delivery system according to one embodiment of the present invention comprising a cassette, barrel and plunger.

FIGS. 29A and 29B are schematic perspective views of one embodiment of a needle with an expandable distal tip for orthopedic device delivery.

FIGS. 30A and 30B are schematic side views of one embodiment of a balloon to assist in orthopedic device delivery.

FIGS. 31A to 31D are partial cut-away schematic side views of an orthopedic device delivery system according to one embodiment of the present invention comprising a plunger, a loading device and a cannula.

FIG. 32A is a schematic rear view orthogonal to FIG. 31A of an embodiment of a knob configured to work with the loading device of the embodiment of the orthopedic device delivery system of FIG. 31A.

FIG. 33A is a schematic side view of an embodiment of a knob configured to work with the loading device of the embodiment of the orthopedic device delivery system of FIG. 31A.

FIGS. 34A to 34C are partial cut-away schematic side views of an orthopedic device delivery system according to one embodiment of the present invention comprising a cannula, a loading device, and a handle with a pistol grip configuration.

FIGS. 35A to 35C are partial cut-away schematic side views of an orthopedic device delivery system according to one embodiment of the present invention comprising a cannula, a loading device, a delivery knob, and a handle.

FIG. 36A is a schematic front view orthogonal to FIG. 35A of the orthopedic device delivery system of FIG. 35A.

FIG. 36C is a partial cut-away schematic front view orthogonal to FIG. 35A of the orthopedic device delivery system of FIG. 35C.

FIGS. 37A to 37C are partial cut-away schematic side views of an orthopedic device delivery system according to one embodiment of the present invention comprising a cannula, a loading device, a handle and a finger-loop trigger.

FIGS. 38A to 38C are partial cut-away schematic side views of an orthopedic device delivery system according to one embodiment of the present invention comprising a cannula, a loading device, a proximal delivery knob and a handle.

FIGS. 39A to 39B are partial cut-away schematic side views of an orthopedic device delivery system according to one embodiment of the present invention comprising a cannula, a loading device, a delivery knob and a handle.

FIGS. 40A to 40C are partial cut-away schematic side views of an orthopedic device delivery system according to embodiments of the present invention comprising a cannula, a handle and a push-button actuated push rod.

FIGS. 41A to 41C are partial cut-away schematic bottom views of an orthopedic device delivery system according to one embodiments of the present invention comprising a cannula, a handle a loading device and a removable tissue piercing device.

FIG. 42A is a side elevational view of another embodiment of a push delivery instrument for an orthopedic device; FIGS. 42B and 42C are front elevational views of the push delivery instrument in FIG. 42A; FIG. 42D is a longitudinal cross-sectional view of the push delivery instrument in a retracted position.

FIGS. 43A and 43B are schematic superior elevational views of an actuating member and an orthopedic device in an extended position and a post-extension retracted position.

FIGS. 44A and 44B are side and superior elevational views of another embodiment of a push delivery instrument; FIG. 44C depicts the partial deployment of an orthopedic device from the push delivery instrument in FIG. 44B.

FIGS. 45A to 45C schematically depict cross-sectional views of the push delivery instrument and orthopedic device in FIG. 42D during an implantation procedure.

Throughout the figures, the same reference numerals and characters, unless otherwise stated, are used to denote like features, elements, components or portions of the illustrated embodiments. In certain instances, similar reference number schemes are used whereby the reference numerals referred to as “AA” in reference numeral “AAxx” correspond to a figure while the “xx” is directed to similar or interchangeable features, elements, components or portions of the illustrated embodiments in different figures. In certain instances, similar names may be used to describe similar components with different reference numerals which have certain common or similar features. Moreover, while the subject invention will now be described in detail with reference to the figures, it is done so in connection with the illustrative embodiments. It is intended that changes and modifications can be made to the described embodiments without departing from the true scope and spirit of the subject invention as defined by the claims.

### DETAILED DESCRIPTION

As should be understood in view of the following detailed description, this application is generally directed to systems and methods for minimally-invasive treatment of bone joints, in both medical and veterinary settings (including both small and large animal veterinary medicine). Bone joints contemplated for various embodiments of the orthopedic systems and methods include, but are not limited to, hands (fingers and thumbs, between phalanges, metacarpals and/or carpals), feet (in the toes, between phalanges, metatarsals and/or tarsals), wrists, elbows, shoulders, knees, hips, and the spine (particularly at the neck and lower back). In some embodiments, an orthopedic device comprises a shape memory body that is inserted into the joint space, which may restore proper joint alignment and joint mobility affected by degenerative processes. In some embodiments, the orthopedic device has a generally arcuate or rectilinear configuration, which may enhance self-centering positioning of the orthopedic device when deployed.

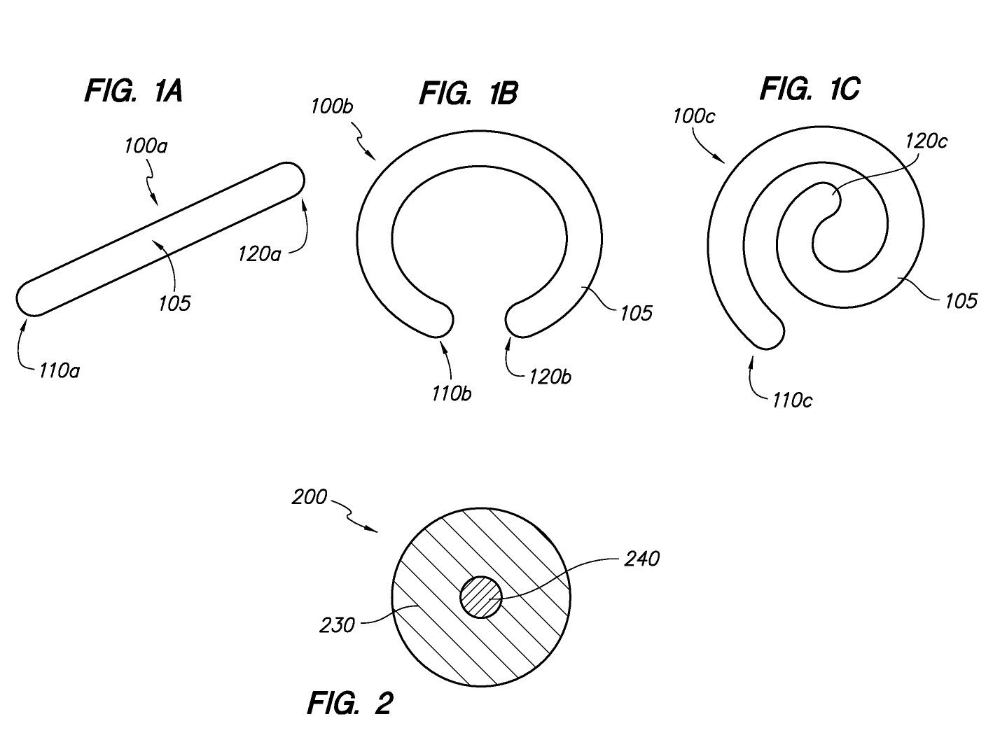

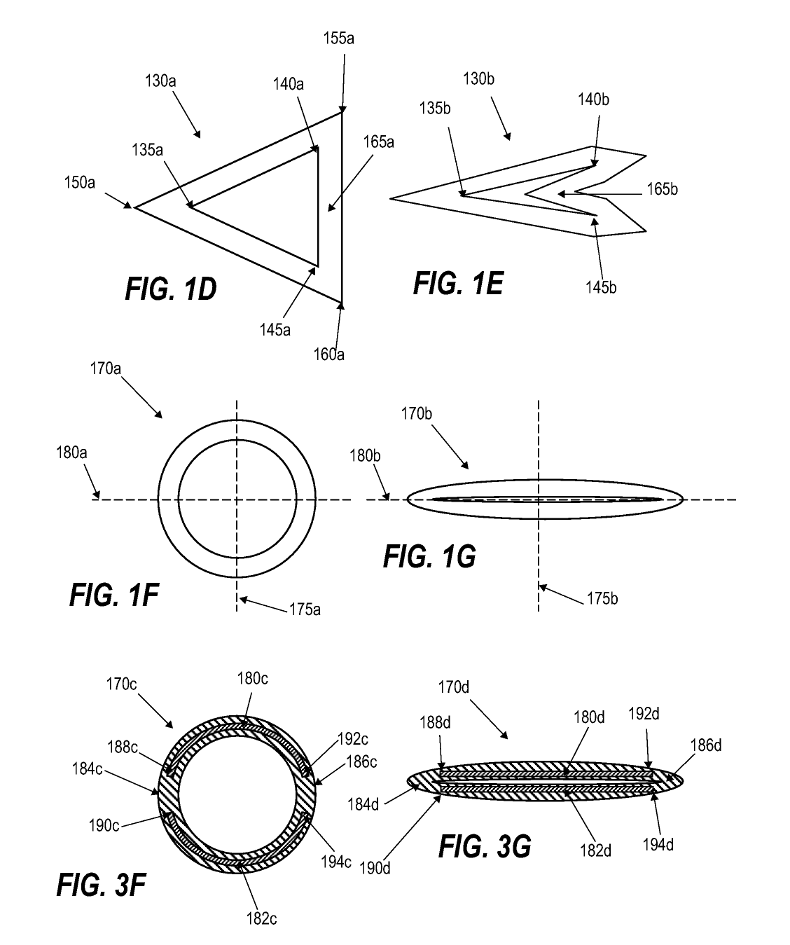

Referring to FIG. 1A, in one embodiment, the orthopedic device 100a comprises a resilient or flexible elongate body 115a with a proximal end 110a and a distal end 120a, and adapted to undergo configurational change. For example, the elongate body 115a of orthopedic device 100a may have a straight configuration as depicted in FIG. 1A, but may also have, for example, an arcuate “C”-shape configuration as shown in FIG. 1B, and/or a spiral-shape configuration in FIG. 1C. The change from one configuration to another may, for example, facilitate implantation of the orthopedic device in a minimally invasive manner, and/or facilitate force redistribution in the joint during movement or positioning.

In one particular embodiment, the distal end 120a of the orthopedic device 100a may be advanced or inserted into the body of a patient first, before the proximal end 110a of the orthopedic device 100a is inserted. In some embodiments, the orthopedic device 100a has a shape or configuration that facilitates its loading into a lumen within a needle, cannula, or other device for delivering the orthopedic device to the implantation site. The straightened configuration of orthopedic device 100a may be used for delivery of the orthopedic device 100a from a substantially straight needle. As the device 100a exits the needle or cannula, the configuration of the device 100a may change to assume the arcuate or spiral configurations of FIGS. 1B and 1C. In another example, the elongate body 115a of the orthopedic device 100a may be bent or biased to a curve to permit delivery from curved or other non-linear needles or cannulas. Thus, the orthopedic device need not have a linear delivery configuration as depicted in FIG. 1A. The orthopedic device 100a may also be configured with a lumen or one or more apertures to facilitate delivery over a delivery structure, such as a rigid or flexible guidewire. Once implanted into the joint, the orthopedic device may be configured to re-expand to its pre-delivery configuration, or may expand to a different configuration. The deployment configuration may be different, depending upon the base configuration of the orthopedic device, and/or whether the orthopedic device has a resilience or bias to one or more particular configurations. The resulting configuration may also result from anatomical restrictions, for example, resulting from the dimensions of the joint capsule, or the geometry of the articular surface. The deployment configuration in the joint capsule may vary in use, depending upon the position joint, the body position of the patient (e.g. standing or lying down) and other conditions which may alter the forces acting on the joint and the orthopedic device. In one example, an orthopedic device has an arcuate configuration that is less-curved, or has a larger major diameter, than the device as fully deployed in the joint, or has an enlarged configuration with at least one dimension that is larger than the corresponding joint space dimension when deployed in the joint space.

In one embodiment, the orthopedic device is configured and implanted to permit its displacement and/or deformation within the joint. In some instances, the movement and/or deformation facilitates the conformation of the orthopedic device to the natural movement of the bones through the range of motion of the joint. For example, the orthopedic device may be implanted into a joint without any attachment to adjacent tissue and constrained only by the joint capsule and/or ligaments within the joint. Because the device is not fixed in place (e.g. attached to either end of bones in a joint), the device may “float” between the ends of the bones in a joint. In some embodiments, a floating design and implantation procedure may provide a mechanical advantage over that of a fixed-type orthopedic device that is rigidly attached to bone tissue by redistributing forces acting on the joint.

For example, the “open ring,” “hoop” or “coil” configuration, or any “open” embodiment, including open polygons of an orthopedic device, may permit a greater range of deformation than closed structures. An open design may facilitate the distribution of the loading, shearing and/or compressive forces seen by the articulation and/or loading of the joint. Thus, in certain open embodiments of orthopedic devices that are flexible, such as orthopedic device 100b, the open configuration may offer reduced or minimal resistance to shape change. Thus, the orthopedic device 100b can spring open or closed as force is applied to the device or to the joint, but still maintain a bearing, cushion, slidable, or articulate surface. However, orthopedic devices with a closed configuration may also be used and may also have deformation properties.

In some embodiments, the gap between the proximal and distal ends of an orthopedic device with an open configuration could be extended to the entire length of the orthopedic device, e.g. when a device is completely straightened. However, various embodiments of an orthopedic device may be configured with functional operating ranges allow varying degrees of flexion and gap widening to support loads and articulation in the joint. In some embodiments, the functional operating range is based upon the amount of stress and strain that the orthopedic device can undergo with significant plastic change (e.g. less than 5%). In some embodiments comprising a shape memory material such as nickel-titanium, the functional operating range may lie within the range of pseudoelastic deformation of the shape memory component, e.g. a Nitinol core that can undergo strain up to about 8%. In one embodiment, the functional flexion in an open orthopedic device allows for a change in the gap between the open ends of the orthopedic device in situ to flex in a range from about 0.5 to about 6 times or more the distance between the gap when the orthopedic device is in its natural state, either pre-implantation or in situ. In one embodiment, the deformation or flex range is roughly from about 2 to about 6 times or greater the natural gap distance, and in another embodiment the flex range is about 3 to about 5 times greater. In one example, the orthopedic device has a flex range with an upper limit of about 4 times. In one embodiment the functional gap can be as wide as a first dimension, diameter, or width of the over all orthopedic device. Thus, orthopedic device 100b may allow for the redistribution of the compressive and/or shearing forces, as well as the resulting wear along the device. In certain embodiments, the orthopedic devices comprise arcuate configurations, such as an open circle or continuous spiral configurations, rather than closed configurations like a complete ring or closed circular shape. The open configurations may result in increased dissipation or redistribution of loading and compression forces though at least one or two deformations in the orthopedic device. First, an open ring allows for dynamic loading response as force that is applied to the joint is partially dissipated by the force necessary to radially-outwardly deform the open ring or spiral into a larger radius profile. In one embodiment, the operating range of radial deformation of an arcuate orthopedic device is in the range of about 0 to about 50% of the orthopedic device profile diameter within the joint. Second, as discussed above, the compression of the articular layer may result in cross-sectional deformation into a flatter shape, which may also dissipate force or pressure in the joint.

In one embodiment, the orthopedic device 100b is sized to snugly fit into the joint capsule itself. In some specific embodiments, one or more portions of the orthopedic device is sized and/or configured to conform to the dimensions of the joint capsule. This fit may facilitate the seating or centering of the orthopedic device 100b with respect to the axis of the bones of the joint, such as in a proximal or distal interphalangeal (PIP/DIP) joint of a finger or an MCP joint of a knuckle.

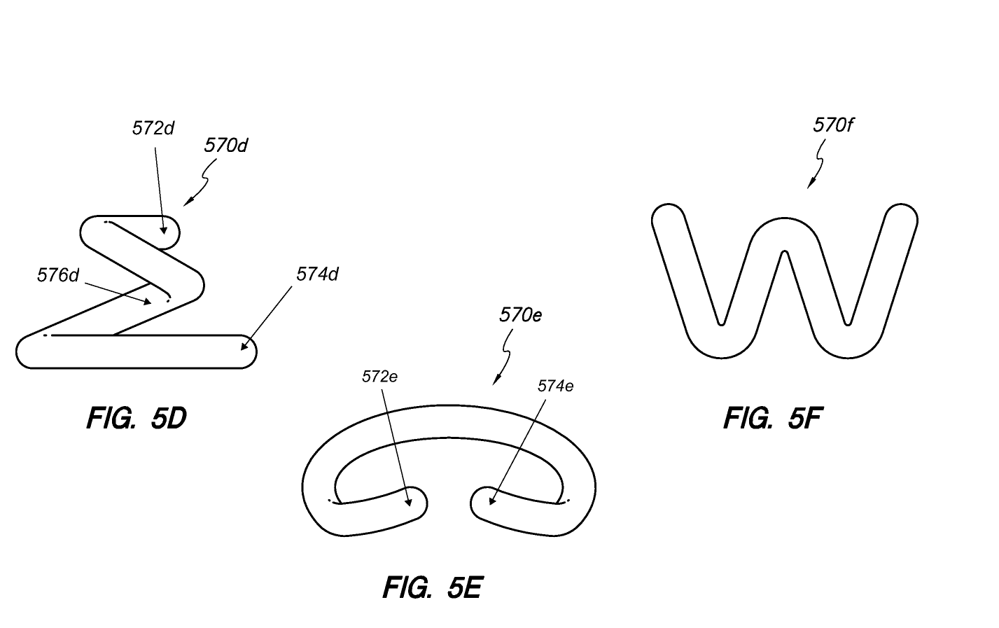

As used herein, “arcuate” may refer to curved or rounded configurations or shapes, but can also include generally arcuate configurations and shapes that have some straight aspect or element with curved or rounded configurations or shapes. As used herein, arcuate and generally arcuate shapes can include open or closed “C”, “O”, “S”, spiral, nautilus, “Q” and other generally arcuate shapes which can be planar or non-planar. Certain embodiments of the orthopedic device may have open or closed rectilinear configurations, which can include polygons such as triangles, squares, rectangles, diamonds, rhombuses, pentagons, hexagons, octagons and other shapes with generally straight edges, and further including shapes and configurations that are generally rectilinear having some curved edge or corners or segments among rectilinear shapes. As used herein, rectilinear and generally rectilinear shapes can include “N”, “M”, “W”, “Z”, “T”, “Y”, “V”, “L”, “X” and other generally rectilinear shapes. FIG. 5F, for example, depicts an embodiment of a rectilinear orthopedic device 570f, comprising a “W”-shape configuration. Various embodiments of generally arcuate or generally rectilinear shapes can include shapes with both rectilinear and arcuate portions, such as a “P”, “R”, “B”, and “U”.

Embodiments of the orthopedic device may have three major dimensions, which can correspond to a first major dimension, a second major dimension and a third major dimension. In one embodiment, the first major dimension, second major dimension and third major dimension correspond to a width, a height and a thickness. Certain embodiments have a thickness which corresponds to the smallest dimension, which may generally correspond to the spacing between articulating surfaces of tissue such as bone or cartilage in a joint. In one embodiment, the width and height can be the same, such as with a circular or square-shape orthopedic device. In other embodiments, the height and width may be different, as with an oval shape or a rectangle or other shape with non-equal height and width. In some embodiments, the orthopedic implant can be implanted in joints of varying sizes, in which the first major dimension and second major dimension may have a range of about 0.0394 to about 4.0 inches (or about 1.0 to about 101.6 mm) and the third major diameter may have a range of roughly about 0.001 to about 0.50 inches (or about 0.025 to about 15 mm). Orthopedic devices having other dimensions may also be used, including but not limited to orthopedic devices configured for larger joints such as the knee, hip, ankle, and shoulder, for example.

As mentioned previously, certain embodiments of the orthopedic device may have a narrowed configuration or a reduced profile to fit in a lumen of a delivery tube or delivery device, or through a small opening in a joint capsule. In one embodiment, a narrowed configuration comprises the reduction of the first major dimension, second major dimension or third major dimension, or a combination thereof. In some embodiments with narrowing configurations, one or more dimensions are reduced while one or more other dimensions are increased. In one embodiment, the orthopedic device can be moved into a narrowed configuration by pinching, squeezing or restraining the device so that parts of the orthopedic device overlap, such as a “C”-shape body being collapsed into an alpha shape (α), a gamma shape (γ), a twisted shape, a helix, and/or a multi-planar configuration, as illustrated in the embodiments of FIGS. 5D and 5E, for example. In one embodiment, the orthopedic device may be manipulated into a straightened or a substantially straightened configuration. In one embodiment, the orthopedic device may have a substantially straightened configuration, including a completely straightened, linear configuration, as well as configurations in which at least a part of the orthopedic device is straightened or partially straightened, configurations in which arcuate orthopedic devices can be made less-arcuate and configurations in which rectilinear orthopedic devices can be made less-rectilinear.

Referring back to FIG. 1A, some embodiments of the orthopedic device may have a relatively uniform width or diameter along its elongate length. However, in other contemplated embodiments, the width of the device body can vary along its length. For example, some orthopedic devices may have one or more tapered sections along a portion of its length, or be tapered along the device's entire length. The tapered section may have a linear or a non-linear taper configuration, and embodiments with two or more tapered sections need not taper in the same direction. The width, or other dimensions of the orthopedic device, can vary from large to small or small to large, making the device thicker in some portions than in others. In one embodiment, the device may be radially compressible along part or over the entire length of the device. In one embodiment, the device may be compressed such that its cross-sectional area is reduced, so that the device may exit a delivery system and expand to a larger cross-sectional area. In one embodiment, the device can be axially compressed or axially stretched along part or over the entire length of the device.

In one embodiment, the orthopedic device 100a comprises a shape memory material. For example, the shape memory material can be made from a heat set or heat-shape shape-memory material, such as Nitinol, or a shape memory plastic, polymeric, or synthetic material, such as polycarbonate urethane. One example of this type of a polyurethane or polyurethane-urea polymer shape memory material is described in United States Patent Publication 2002/0161114 A1, which describes a shape memory polyurethane or polyurethane-urea polymer including a reaction product of: (A) (a) silicon-based macrodiol, silicon-based macrodiamine and/or polyether of the formula (I): A-[(CH2)m-O-]n-(CH2)m-A′, wherein A and A are endcapping groups; m is an integer of 6 or more; and n is an integer of 1 or greater; (b) a diisocyanate; and (c) a chain extender; or (B) (b) a diisocyanate: and (c) a chain extender, where the polymer has a glass transition temperature which enables the polymer to be formed into a first shape at a temperature higher than the glass transition temperature, and where the polymer is maintained in the first shape when the polymer is cooled to a temperature lower than the glass transition temperature, so that the polymer is capable of resuming its original shape on heating to a temperature higher than the glass transition temperature. Various embodiments may include a shape memory polymer alone, or a blend of two or more of the shape memory polyurethane or polyurethane-urea polymers or at least one shape memory polyurethane or polyurethane-urea polymer defined above in combination with another material. Other embodiments relate to processes for preparing materials having improved mechanical properties, clarity, processability, biostability and/or degradation resistance and devices or articles containing the shape memory polyurethane or polyurethane-urea polymer and/or composition defined above.

In other embodiments, the orthopedic device may comprise any of a variety of rigid, semi-rigid or flexible materials, which may be metallic or non-metallic, polymeric or non-polymeric, bioresorbable or non-bioresorbable, lipophilic, hydrophilic or hydrophobic, for example. These materials may include but are not limited to stainless steel, cobalt-chromium, titanium, pyrolytic carbon, any of a variety of ceramic or hydroxyapatite-based materials, polymers such as PTFE, silicone, nylon, polyethylene, polypropylene, polycarbonate, polyimide, polycarbonate, polyurethane, PEEK, PEKK and PEBAX, any of a variety of bioresorbable materials such as PGA, PLA, PLGA, PDS and the like, as well as chitosan, collagen, wax and alginate-based materials, and animal-derived materials such as small intestine submucosa (SIS).

In one embodiment, the orthopedic device 100a comprises an articular layer 105, blanket or jacket. The articular layer 105 is sized and configured to be placed within a body, such as in a joint, as a layer between bones of the joint to provide a slidable articulation surface and/or a cushion. In some embodiments, the articular layer can range from about 0.001 to about 0.5 inches thick (or about 0.025 to about 13 mm). The orthopedic device may or may not include a core, backbone or other support structure, which may support the articular layer or contribute or impart certain features or characteristics to the orthopedic device. Support structures, such as the core, are described in greater detail below.

In one embodiment, the articular layer 105 is configured to be compressed by forces acting on the joint. For example, in one embodiment an articular layer may be compressed from a substantially circular cross-sectional shape to an oval, elliptical, or football shaped cross-sectional shape. As the compression occurs, the amount of surface coverage of the articular layer with respect to bony joint contact, resulting in reduced in relative pressure across the joint. In one embodiment, the operating range of compression of an orthopedic device is in the range of about 0 to about 50% of the cross-sectional diameter or other dimension along the axis of compressive force.

The articular layer 105 may comprise one or more layers of material, and any of a variety of materials may be used for each layer. In certain embodiments of the orthopedic device 100a, the body of the orthopedic device 100a comprises an articular layer with shape-memory properties, with or without any backbone or other type of support structure. The shape-memory properties may include but are not limited to temperature-induced configuration changes as well as stress-induced pseudoelastic properties. In certain embodiments, the articular layer 105 materials may include but are not limited to silicone, PTFE or ePTFE, ultra high molecular weight polyurethane or and any implantable grade material, or other materials disclosed above. The articular layer 105 can be compliant and/or compressible, or may have a non-compressible construction. In certain embodiments, the articular layer 105 can have any of a variety of durometers (material hardness) from about 30 to about 90 Shore A, for example. In certain embodiments, the articular layer 105 may comprise a porous material, which may have a closed or open-pore structure. The porous coatings, layers or structures may include but are not limited to macroporous or nanoporous coatings or structures. In some instances, a porous coating may facilitate tissue ingrowth and/or augment the inflammatory response to the orthopedic device, if any. In another embodiment, the coating material can form a casing (or covering) that is spongy or harder or less compliant. The pores of the material could be loaded with one or more therapeutic agents. The casing could form a scaffold for tissue ingrowth and could be used in joints with certain wear characteristics, but is not limited to use with these joints. In some embodiments, the articular layer 105 may be coated with a secondary surface layer, such as another polymer of a different material property, or an anti-friction high wear material such as Parylene, or other similar materials which are known to the art as providing for a low friction surface.

In certain embodiments, the articular layer 105 may contain a material or a drug to inhibit or promote inflammation, joint deterioration etc., or a material or drug to encourage tissue regeneration or device encapsulation. For example, certain embodiments of the articular layer 105 may be coated with or contain one or more therapeutic agents, such as a long-acting steroid or a disease-modifying anti-rheumatic drug (DMARD). DMARDs include but are not limited to agents such as gold, D-penicillamine, methotrexate, azathioprine and cyclophosphamide, leflunomide, etanercept, infliximab, minocycline and certain anti-malarial agents used for arthritis treatment, for example. The therapeutic agents need not be limited to joint-specific therapy agents, however. In other embodiments, the therapeutic agent may include an antibiotic (e.g. a macrolide, a cephalosporin, a quinolone, an aminoglycoside, a beta-lactam or beta-lactamase inhibitor, a lincosamide, or glycopeptides antibiotic, etc.), a sclerosing agent (e.g. bleomycin, tetracycline, talc, alcohol, sodium tetradecyl sulfate, etc.), or other type of inflammation-inducing agent, a growth factor (e.g. connective tissue growth factor, cartilage-derived retinoic acid sensitive protein), and other agents. In some embodiments, one or more therapeutic agents may be injected or infused into a joint space, separate from the orthopedic device, using any of a variety of forms (aqueous solution, suspension, oil, foam, a separate drug eluting disc or other structure, etc.).

In other embodiments, the articular layer may comprise a plurality of surface projections and/or pores, which may cause a mechanical irritant response when implanted and may induce an inflammatory response. The projections and/or pores may be grossly visible on the surface of the articular layer, or may be nano- or micro-sized structures. The projections may comprise discrete surface structures or aggregated structures, including but not limited to hooks, barbs, tubes, rods, cones, spheres, cylinders, loops, pyramids, or a mix thereof. These structures may have a size in the range of about 5 nm to about 5 mm or more, sometimes about 50 nm to about 3 mm, and other times about 500 nm to about 1 mm, and in still other times about 1 μm to about 500 μm.

In one embodiment the coating and or covering can be used to stimulate a thrombotic or coagulant response, and/or organization of tissues or fluids it contacts. For example, the coating or covering may comprise a hemostatic agent such as chitosan, zeolite, fibrinogen, anhydrous aluminum sulfate, titanium oxide, one or more clotting factors or other constituents of the blood clotting cascade.

In some embodiments, one or more therapeutic agents may be mixed with a polymer material which may either biodegradable or non-biodegradable. Thus, release of the therapeutic agents may occur by elution from the polymer material, and/or by degradation of the polymer material. For example, an orthopedic device may comprise a material or reservoir being drug loaded and dissolvable through features provided in a jacketing or coating material, such as through micro holes, pores, or some other feature. In certain embodiments the articular layer is provided with reservoirs, depots, cavities, wells, pockets, porous materials, bubbles or capsules for drug delivery. In one specific example, the orthopedic device could be a drug-loaded element that slowly dissolves to elute a drug of some sort through a casing that is spongiform or porous. This would leave behind the casing after the ring has dissolved. In some embodiments, timed drug delivery could be configured for more controllable dosing. For example, about 75% to about 90% of a therapeutic agent may be released or dissolved over a timeframe of anywhere from about 4 hours to about 4 months or more, sometimes from about 24 hours to about 6 weeks or more, other times from about 72 hours to about 4 weeks, and still other times from about 2 weeks to about 4 weeks. In other embodiments, the casing would maintain the space filling or cushioning feature desired and/or allow for tissue organization or in-growth.

The therapeutic agent may be provided on an outer surface or an inner surface of the articular layer, or within a volume or layer of the articular layer. As mentioned previously, the articular layer may comprise one or more rate control layers to alter the rate of therapeutic agent release. The rate control layer may comprise, for example, polymer layers with a reduced permeability or smaller pore structure.

In one specific embodiment, a coating may comprise a xenograft, allograft or autograft biological covering, from a live and/or cadaveric donor, or a biological material grown from a tissue culture. For example, tissue harvested directly from the patient could be harvested using a laparoscope or other tissue removal and collection system and then affixed to the core, articular layer, preshaped ring or backbone and secured to the orthopedic device. The tissue may include but is not limited to omental tissue, ligamentous or tendinous tissue, cartilage tissue, bone tissue and the like. The graft material may retain the native tissue structure or may have undergone additional mechanical processing (e.g. crushing, blending, etc.) or biological processing (treatment with glutaraldehyde or other cross-linking agents, sterilization with electron-beam, gamma irradiation or ethylene oxide, etc.) The device could then be loaded into a delivery cannula and inserted and ejected (deployed) in the same fashion as the delivery systems employed and described herein. In some embodiments, the articular layer 105 comprises a cartilage replacement material, or a natural or synthetic cartilage.

In another embodiment, an orthopedic device is covered with a material, biological agent, or other coating that expands in volume with contact to fluids. The fluids may be the endogenous fluid found in the joint itself, and/or externally added fluids. Expandable materials may permit the insertion of a device of a diameter that is smaller than the fully expanded finished diameter. For example, a coating on the backbone or the articular layer could be hydrophilic in that it could transition from one configuration or diameter (small for insertion) to a larger configuration or diameter when contacting either the body fluid or some fluid provided from an outside source, such as saline.

In one specific embodiment, the expandable or swellable covering may comprise a composite or matrix with a polymer and a biological material i.e. tissue, including but not limited to cartilage, collagen, ligaments, muscle, etc. In one embodiment, the scaffold could be a polymer-based material. In various embodiments, the casing or covering of the orthopedic device is configured to swell from the small insertion dimension or diameter after implantation to a larger finished dimension or diameter. In some alternate embodiments, such as those disclosed in U.S. application Ser. No. 12/099,296, filed Apr. 8, 2008, the orthopedic device may comprise an inflatable structure. The inflatable structure may be inflated with a gas, liquid, gel, or slurry which may or may not be curable to a solid state. The inflatable structure may also be expanded by filling the structure with a volume of solid structures, such as microspheres or other small structures.

In certain embodiments, the articular layer 105 is radiopaque, and can augment the visibility of the device when implanted as viewed by X-ray and/or fluoroscopic equipment. In one embodiment, the radiopacity of the articular layer 105 is provided by radiopaque markers or structures (not shown here) on or embedded in the layer 105, or by loading or doping the articular layer 105 with platinum, gold or other biocompatible metal.

In various embodiments, any of the features of the articular layer or coatings mentioned herein may be combined on the orthopedic device, either as different layers of the orthopedic device or as different sections or regions of the orthopedics device. In one embodiment, an articular layer or coating can provide for tissue ingrowth or fusion with bone, cartilage, or other tissue while another surface provides a low-friction surface to another side of the joint. Any combinations are possible. In some embodiments, adhesives or transitional polymer layers may be provided to facilitate the attachment of two or more other layers of the articular layer.

As described previously, the orthopedic device can have an arcuate, rectilinear or non-straightened configuration once it is implanted in a joint. Some non-limiting examples of arcuate configurations include an open ring (also called an open hoop or an open loop) such as is shown in the embodiment in FIG. 1B, and a nautilus-style spiral as is shown in the embodiment in FIG. 1C. Referring to FIG. 1B, the open hoop arcuate configuration of the orthopedic device 100b has a proximal end 110b and a distal end 120b in relation to insertion into the body of a patient, such as into a joint. In certain embodiments, the orthopedic device 100b of FIG. 1B may have similar attributes and characteristics of the orthopedic device 100a of FIG. 1A, such as shape memory and/or an articular surface 105. In certain embodiments, orthopedic device 100b is an arcuate configuration of orthopedic device 100a. In certain embodiments, the orthopedic device 100a is biased to the configuration as shown for orthopedic device 100b. The bias may be a preferred configuration for a flexible, pliable, bendable device. In certain embodiments, the orthopedic device of 100a may change to from one configuration to another (e.g. from the configuration of orthopedic device 100a in FIG. 1A to the configuration of orthopedic device of 100b in FIG. 1B) by a change in ambient or implantation site temperature, by a release from deformation stresses, or by the introduction of an activating medium or material. In certain embodiments, the orthopedic device is reversibly configurable between various shapes or geometries.

As mentioned previously, the orthopedic device may also comprise a closed shape that forms a complete perimeter along at least one section or portion of the device. In FIG. 1D, for example, the orthopedic device 130a comprises an expanded configuration with a closed triangular shape. Although the triangular shape in FIG. 1D comprises an equilateral triangular shape with uniform angles 135a, 140a and 145a, in other embodiments, one or more angles may be different from the other angles. Also, although the inner angles 135a, 140a and 145a, along with outer angles 150a, 155a and 160a have sharp angles, in other embodiments, one or more of these angles may be rounded. In its collapsed state, depicted in FIG. 1E, the inner angles 135b, 140b and 145b of the orthopedic device 130b may narrow to collapse its triangular shape into an arrow shape. The orthopedic device 130b may also have a bending section 165a that collapses from a straight configuration to a bent configuration to facilitate the reduction in the cross-sectional profile of the orthopedic device 165b. Although the orthopedic device 130b is depicted as generally collapsing within the plane of the orthopedic device 130a in its expanded configuration, in some embodiments, this and other orthopedic devices disclosed herein may also fold onto themselves or otherwise collapse out of plane to reduce their cross-sectional profile. In other embodiments, the orthopedic device may comprise other polygonal shapes or curvilinear shapes, with angles and/or sides that may be uniform or different, with angles that narrow or widen when changing from one configuration to another configuration. Although several embodiments described herein have a base configuration that is the expanded or deployed configuration, in other embodiments, the base configuration may be the delivery configuration. In still other embodiments, the orthopedic device may comprise a malleable or plastic material or structure with any bias toward one or more configurations.

FIGS. 1F and 1G illustrate another embodiment of an orthopedic device 170a/170b comprising a closed arcuate configuration. In its deployed configuration, the orthopedic device 170a comprises a closed circular configuration, but other embodiments, may comprise an oval or ovoid shape (e.g. one end being larger than the other end). To transform the device 170a to its delivery configuration, the orthopedic device 170a shortens along a first dimension 175a while lengthening along a second dimension 180a. In some embodiments, the delivery axis of the orthopedic device 170b may be transverse to the first dimension 175b, or parallel to the second dimension 180b.

One example of a nautilus-style spiral arcuate configuration is the embodiment of an orthopedic device 100c as shown in FIG. 1C. The orthopedic device 100c has a proximal end 110c and a distal end 120c in relation to insertion into the body of a patient, such as into a joint. In certain embodiments, orthopedic device 100c has many similar attributes and characteristics of orthopedic device 100a and/or 100b, such as shape memory and/or an articular surface 105. In certain embodiments, orthopedic device 100b is an arcuate configuration of orthopedic device 100a. In certain embodiments, the orthopedic device of 100a may be altered in to a configuration as shown for orthopedic device of 100c. The bias may be a preferred configuration for a flexible, pliable, bendable device. In certain embodiments the orthopedic device 100a, when unconstrained, can change to the configuration as shown for orthopedic device of 100c, or by a change in ambient or implantation site temperature or the introduction of an activating medium or material. In certain embodiments, the orthopedic device is reversibly configurable between various shapes or geometries.

In some embodiments, the orthopedic device is configured to float inside the joint, which may better conform to the natural movement of the bones through the range of motion of the joint. The nautilus-style spiral arcuate configuration depicted in FIG. 1C, for example, may also offer certain advantages described for the open hoop arcuate configuration, or hoop configuration, but also provides a larger bearing surface to the joint. With the extended length of the spiral configuration, the orthopedic device 100c is configured to provide more of an articulate surface, which may result in decreased pressure on the bones by dissipating forces over a larger surface area. The cross-sectional diameter multiplied by the number of winds in a spiral shape roughly equals the surface area coverage of the articular surface in conformation with the bones of the joint. For example, a small cross-sectional diameter of a spiral configuration allows for a plurality of windings in the spiral. This plurality of spiral windings can then adjust to the general surface area of either bone as the joint articulates.

As noted previously, some embodiments of the devices can have additional structures within it. For example, in FIG. 2 an orthopedic device 200 comprises an elongate core 240 and an articular layer 230 surrounding at least a portion of the core 240. Referring back to FIGS. 1A to 1C, various embodiments of orthopedic devices 100a, 100b and/or 100c can either have an elongate core or lack an elongate core. Other embodiments of orthopedic devices 100a, 100b and/or 100c may also either have an articular layer or lack an articular layer. Thus, the orthopedic device may consist of an elongate core, an articular layer, or both. In various embodiments directed to use in PIP, DIP and MCP joints, for example, the cross-sectional diameter or thickness of a core can range from roughly about 0.001 to about 0.50 inches (about 0.025 to about 15 mm) with some embodiments in a range of roughly about 0.005 to about 0.015 inches (about 0.13 to about 0.38 mm), and some embodiments in a range of roughly about 0.01 to about 0.0125 inches (about 0.26 to about 0.32 mm). In various embodiments, the cross-sectional outer diameter or overall thickness of an articular layer can range from roughly about 0.003 to about 0.50 inches (about 0.076 to about 12.7 mm) with some embodiments in a range of roughly about 0.039 to about 0.118 inches (about 1 to about 3 mm), and some embodiments in a range of roughly about 0.078 to about 0.098 inches (about 2 to about 2.5 mm). In some embodiments a ratio of core cross-sectional diameter (or thickness) to articular layer cross-sectional outer diameter (or thickness) can range from about 0 to about 500, and in other embodiments may have ranges of ratios from about 2 to about 30. Other dimensions with the same, similar or different ratios can be used in other parts of the patient's body. Orthopedic devices with cores having other dimensions may also be used, including but not limited to orthopedic devices configured for larger joints such as the knee, hip, ankle, and shoulder, for example.

As illustrated in the embodiment of FIG. 2, the orthopedic device 200 includes the elongate core 240 in addition to the articular layer 230. In some embodiments, the articular layer 230 surrounds, encapsulates, encloses or covers at least a portion of the core 240. In some other embodiments, the articular layer 230 can surround or encapsulate the entire elongate core 240. As used herein, “surround,” “encapsulate” and “enclose” include configurations in which a core is not completely surrounded, completely encapsulated or completely enclosed. For example, certain embodiments of an orthopedic device contemplate an articular layer which “surrounds” an elongate core with a continuous or non-continuous helical band, discontinuous tabs, or other intermittent articular layer structure.

In some embodiments, the articular layer 230 may have some or all of the features of other articular layer embodiments described herein. In one embodiment, the ratio of the cross-sectional size of the elongate core 240 to the articular layer 230 is in the range of about 10:1 to 1:10, sometimes in the range of about 5:1 to about 1:5 and other times with a ratio of about 2:1.

In one embodiment, the elongate core 240 comprises a shape memory material. The shape memory material may be made from a heat set/shaped shape-memory material, such as Nitinol, or a shape memory plastic, polymeric, synthetic material. For example, one embodiment of the elongate core 240 comprises a shape memory material including a shape memory polyurethane or polyurethane-urea polymer, as described above. In one embodiment the elongate core 240 comprises a metal “open” ring such as Nitinol encapsulated by an articular layer 230, or outer blanket, comprising silicone. In one embodiment the elongate core 240 comprises a hardened polymer. In one embodiment, the elongate core 240 is configured such that a heat set Nitinol with an arcuate configuration, such as an open ring configuration, a horseshoe configuration, or a spiral configuration, can be straightened for delivery through cooling or plastic deformation, then recovered to its original heat-set shape once released from a delivery system, such as one embodiment using a properly sized hypodermic needle. In one embodiment the elongate core 240 comprises a non-shape memory material which can be bent or deformed.

In certain embodiments, the elongate core 240 is coated or impregnated with a drug or other therapeutic agents as described previously with respect to the articular layer. The therapeutic agents of the elongate core 240 may be the same or different from the therapeutic agents of the articular layer or other layers or coatings of the orthopedic devices.

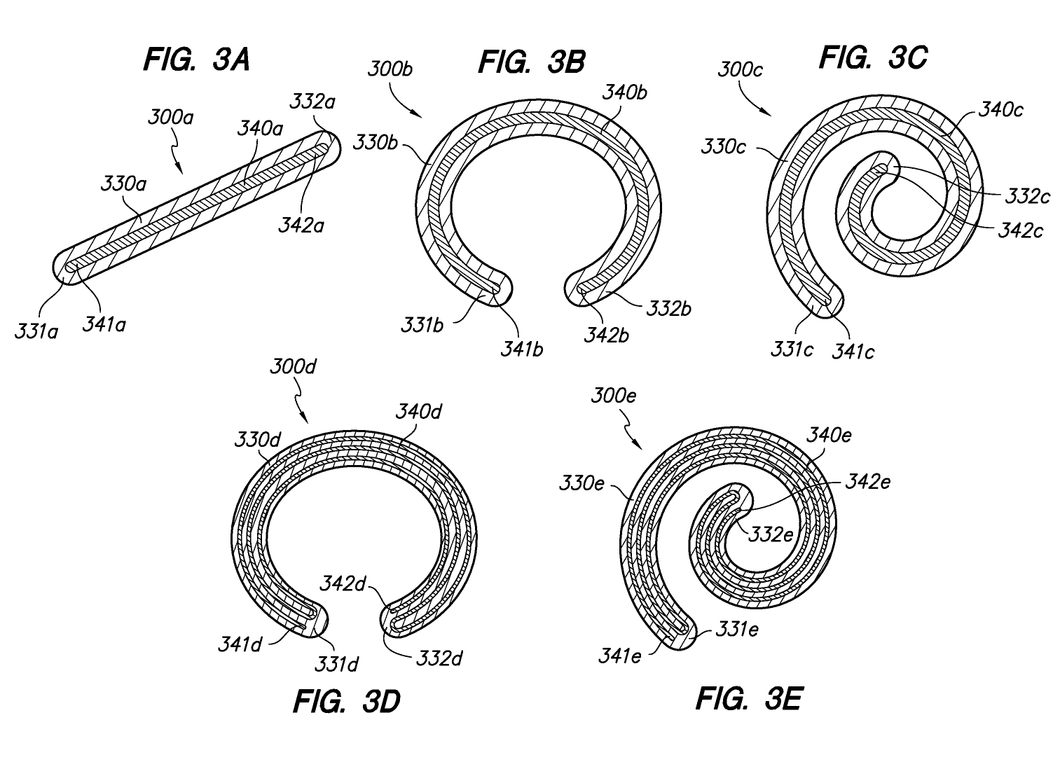

FIGS. 3A to 3E are longitudinal cross-sectional views of the orthopedic devices 100a-100c in FIGS. 1A to 1C with various configurations of optional support structures or cores. FIG. 3A is a schematic cross-sectional view of an orthopedic device 300a comprising a substantially straightened configuration. In this embodiment, the device comprises an elongate core 340a and an articular layer 330a surrounding at least a portion of the core 340a. The articular layer 330a has a proximal end 331a and a distal end 332a. The elongate core 340a has a proximal end 341a and a distal end 342a. In one embodiment, the orthopedic device 300a may be a cross-sectional view of the orthopedic device 100a described above, with a core 340a. FIG. 3B shows a device an elongate core 340b and an articular layer 330b surrounding at least a portion of the core 340b in an open hoop arcuate configuration. The articular layer 330b has a proximal end 331b and a distal end 332b, while the elongate core 340b has a proximal end 341b and a distal end 342b. In one embodiment, the orthopedic device 300b may be a cross-sectional view of the orthopedic device 100b described above. Certain embodiments of a spiral shaped device, such as is shown in FIG. 3C can have a single elongate core. For example, orthopedic device 300c comprises a nautilus-style spiral arcuate configuration, the device comprising an elongate core 340c and an articular layer 330c surrounding at least a portion of the core 340c, the articular layer 330c comprises a proximal end 331c and a distal end 332c, and the elongate core 340c has a proximal end 341c and a distal end 342c. In one embodiment, the orthopedic device 300c may be a cross-sectional view of the orthopedic device 100c described above.

In some embodiments, the elongate core may be wrapped around itself or comprise of a number of distinct or separate sections or segments, as shown in FIGS. 3D and 3E. FIG. 3D shows an orthopedic device 300d with an open hoop arcuate configuration. In one embodiment, the orthopedic devices 300d may be a cross-sectional view of the orthopedic device 100b described above, with an optional folded or overlapping core 340d. The device 300d comprises one or more elongate cores 340d wrapped, braided or folded back along a length of the device, and an articular layer 330d surrounding at least a portion of the core(s) 340d. The articular layer 330d has a proximal end 331d and a distal end 332d. The elongate core 340d in FIG. 3D comprises a unitary body with a proximal end 341d, a distal end 342d, an inner segment 350d, a middle segment 352d and an outer segment 254d. The segments 350d to 354d may be interconnected as depicted in FIG. 3D, but in other embodiments may one or more segments may be separated. The segments of the embodiments described herein may themselves have subsegments, e.g. the inner segment 350d may comprise a proximal segment and a distal segment. Also, the segments of a core may generally have a similar length, such as segments 350d to 354d in FIG. 3D, but one or more segments may also have a different length In some embodiments for example, two or more elongate cores 340d are situated in a roughly parallel or co-linear orientation, which can be twisted or braided or interlocked. Other embodiments of the orthopedic device need not be limited to a single elongate core or backbone, but may have a plurality of cores or backbones including a braided configuration, continuous overlaps, etc. FIG. 3E shows an orthopedic device 300e with a nautilus-style spiral arcuate configuration. In some embodiments, the orthopedic device 300e may be a cross-sectional view of the orthopedic device 100c described previously, but with an optional folded or overlapping core 340e. The device 300e comprises one or more elongate cores 340e wrapped or folded along a length of the device and an articular layer 330e surrounding at least a portion of the core(s) 340e. Although the core 340e generally extends from one end 331e of the orthopedic device 300e to the other end 332e, in other embodiments, the core 340e may extend out from the articular layer 330e at either or both ends 331e, 332e of the orthopedic device 300e, or anywhere between the two ends 331e, and 332e. In other embodiments, the core 330e may have a length that is substantially less than the length or the orthopedic device 300e. For example, the core may be provided only along the outer spiral portion of the orthopedic device, leaving the inner overlapping portion of the orthopedic device with a portion of the core. In other embodiments, only the inner portion of the orthopedic device may comprise a core, while the outer overlapping portion lacks a core.

FIGS. 3F and 3G depict one embodiment of the orthopedic device 170a and 170b depicted in FIGS. 1F and 1G configured with one or more optional cores. As shown in FIG. 3F, in the orthopedic device 170c in the expanded configuration comprises two separate cores 180c and 182c. In other embodiments, the orthopedic device may have a single core, or three or more cores, including but not limited to four cores, five cores, or six cores, for example. The cores may have substantially similar lengths or their lengths may be substantially different. The cores may also have substantially similar or different cross-sectional or elongate shapes. In some embodiments, the cores 180c and 182c may be separate but arranged in contact with each, or they may be separated by non-core sections 184c and 186c at one or both ends 188c, 190c, 192c and 194c of the cores 180c and 182c. In some embodiments, the non-core portions of an orthopedic device may facilitate a particular collapsed configuration. For example, the narrow oval configuration of the orthopedic device 170d in FIG. 3G illustrates how the non-core sections 184d and 186d may permit substantial bending in the collapsed state compared to portions of the orthopedic device 170d along the cores 180d and 182d. In some embodiments, the perimeter or length of the orthopedic device, whether having an open or closed configuration, may comprise a ratio of core to non-core portions in the range of about 0 to about 1, sometimes about 0.3 to about 1, and other times in the range of about 0.7 to about 0.95.

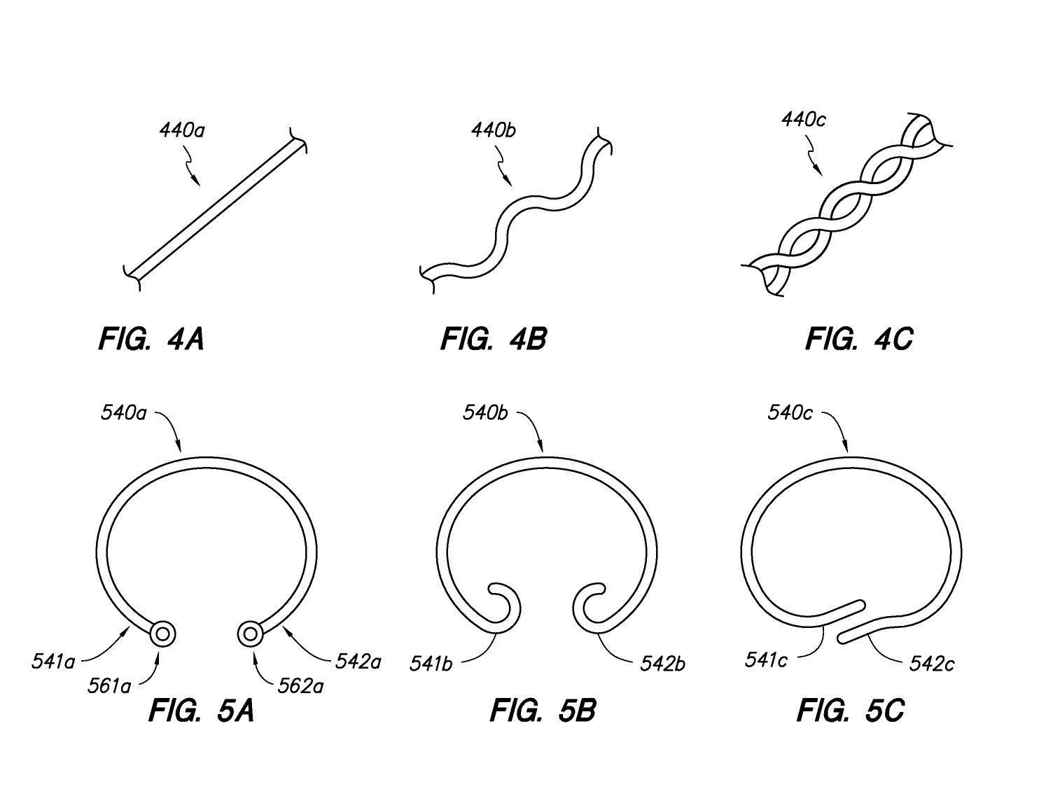

The shape of the elongate core can vary, as is shown in the embodiments of FIGS. 4A to 4C. FIG. 4A shows an elongate core 440a with one or more substantially linear or straight members. FIG. 4B shows an elongate core 440b with one or more wave, curve or zig-zag members that may be in one or more planes at any angle with respect to one another. FIG. 4C shows an elongate core 440c with one or more members in a braided or weave configuration. Any of these patterns can be used with any of the elongate cores disclosed herein.

Various embodiments of elongate cores can have different features along the length or ends of the core, as is shown in FIGS. 5A to 5C. An elongate core 540a with an open hoop arcuate configuration can have one or more end segments, as is shown in FIG. 5A. Such end segments can include proximal end segment 561a and/or distal end segment 562a. In some embodiments, the optional end segments 561a and/or 562a may be configured with an enlarged axial cross-sectional area compared to the portions of the core 540a between the end segments 561a, 562a. The end segments may have any of a variety of configurations, including but not limited to the ring or loop configurations depicted in FIG. 5A. In other embodiments, the end segments may have a T-tag configuration, a spherical or ovoid configuration, a helical or spiral configuration, or any other configuration. The orientation of the end segments may lie within the plane of the rest of the orthopedic device, or may be perpendicular, transverse or some non-planar orientation with respect to the orthopedic device. The configuration of each end segment, if any, may be the same or different. Each end segments may be embedded within the articular layer of the orthopedic device, but in some embodiments, some or all of the end segments may at least partially project from the articular layer or otherwise be exposed with respect to the articular layer. In one particular example, the end segments 561a and 562a of FIG. 5A may be exposed so that the ring configurations may be used to attach a suture or other structure to the orthopedic device. In other embodiments, the end segments 561a and 562a may help to resist relative separation or displacement of the articular layer and the core 540a, and/or to reduce the risk that ends of the core 540a may In various embodiments, the elongate core or cores 540a can have zero, one, two or more end segments. In one embodiment the end segment 561a or 562a is radiopaque or can be used as a marker for visualization of the ends of the orthopedic device. The end segments 561a and 562a may comprise the same or different material as the length of the elongate core 540a. In one embodiment, the end segments 561a and 562a are separate elements made of the same or different material as the length of the elongate core 540a and which are bonded, fused, welded, glued, or otherwise attached to the proximal end 541a and a distal end 542a, respectively.

Although not illustrated, it is contemplated that an elongate core 540a may have one or more medial segments anywhere along the length of the elongate core 540a. In various embodiments, elongate core 540a has end segments or medial segments to help improve stability of an articular layer or outer blanket, and need not be flat or planar, but can be biased out of the primary plane of the device at one end or both ends.

In another embodiment, an elongate core 540b may include one or more bends, such as proximal bend 541b and/or distal bend 542b as shown in FIG. 5B. In some embodiments, the bends may also include hooks. In various embodiments, the bends or hooks can be closed off to form a loop, as with certain embodiments of elongate core 540a. The bends 541b and/or 542b may be generally oriented radially inward, as shown in FIG. 5B, or radially outward. The bends need have the same orientation, however. For example, the elongate core 540c shown in FIG. 5C comprises a proximal segment 541c that is bent radially inward from the curvature of the elongate core 540c and a distal segment 542c that is bent radially outward with respect to the overall configuration of the elongate core 540c. In other embodiments, proximal segment 541c and/or distal segment 542c are bent radially inward, radially outward, and/or up or down from the primary plane of the elongate core 540c. FIG. 5E, for example, depicts an embodiment of an orthopedic device 570e with its ends 572e and 574e oriented out-of-plane in a relative upward direction. The orthopedic device 570e may optionally comprise an arcuate core (not shown) with ends that are bent out-of-plane. Thus, in embodiments, like the core the orthopedic device comprises ends which are biased or bent slightly towards or away from its center, the optional core or support structure of the orthopedic device may be similarly configured. In other embodiments, however, the general configuration of the core and the general configuration of the articular layer or the orthopedic device may be the same or may be different.

FIG. 5D schematically illustrates another embodiment of a non-planar orthopedic device. In this particular embodiment, the portions of the orthopedic device 570d at each end 572d and 574d may have a generally planar configuration, interconnected by a compressible axial member 576d. The axial member 576d may have a multi-angle configuration, as shown in FIG. 5D, but may also comprise a multi-curved or helical configuration, for example. In some embodiments, the planar ends 572d and 574d of the orthopedic device may facilitate the alignment of the orthopedic device 570d with the articulating surfaces of bones of a joint. In some embodiments, the orthopedic device with a multi-planar configuration may augment the shock absorbing characteristics of the orthopedic device, including orthopedic devices that undergo frequent or substantial axial loading, such as a knee joint. Here, the configuration of the axial member 576d may modify the axial loading characteristics of the joint relative to an orthopedic device with a generally planar configuration.

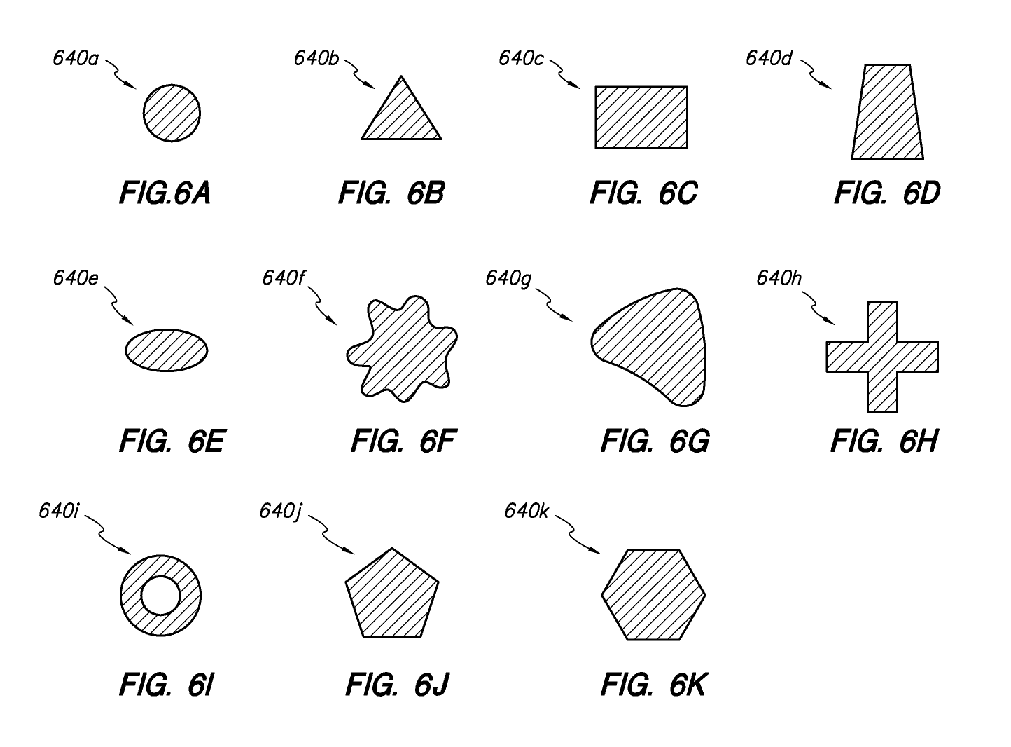

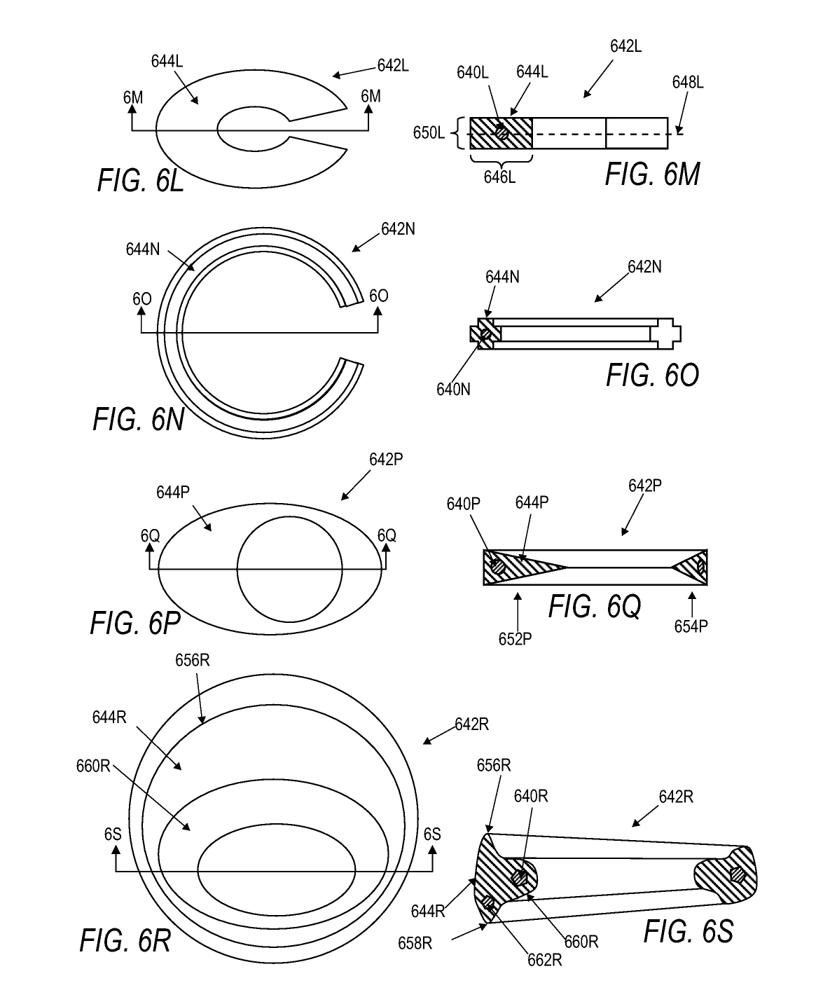

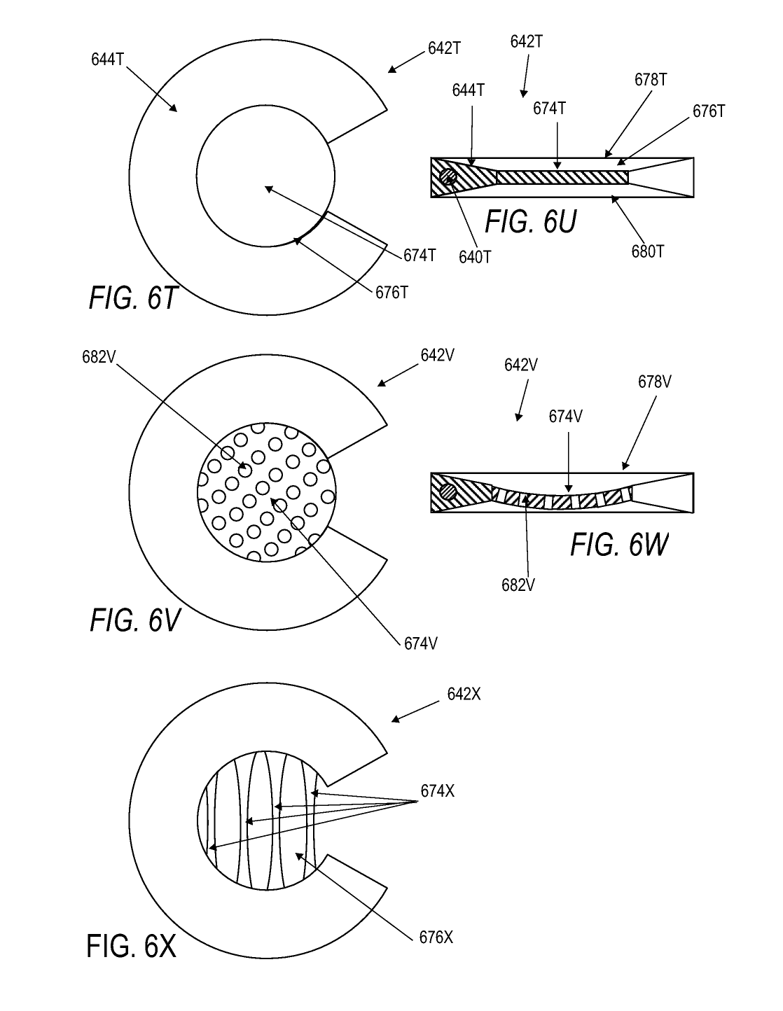

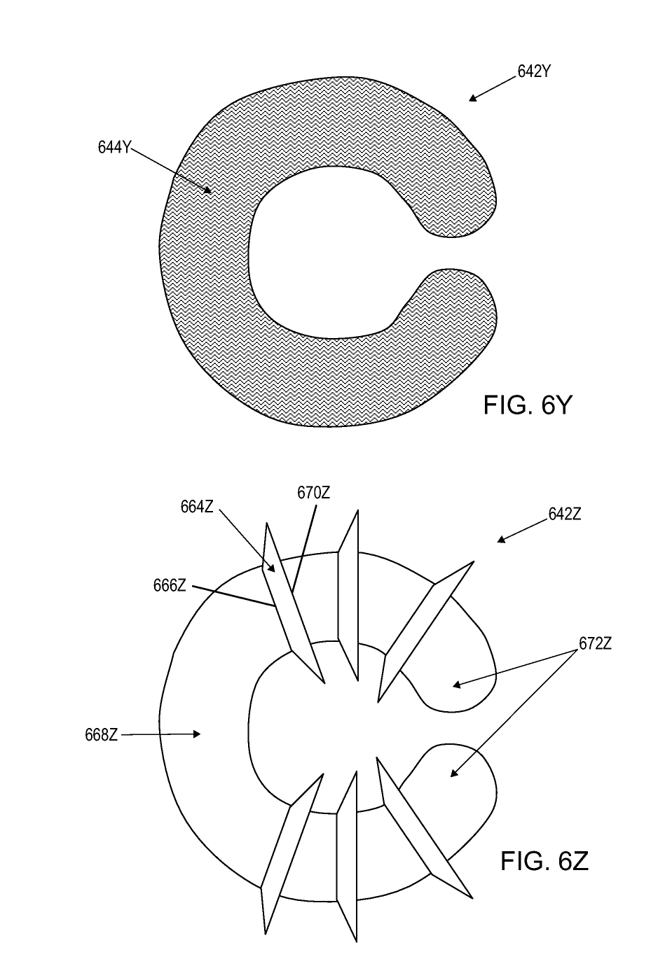

In embodiments of the orthopedic devices comprising elongate cores, the cores may have any of a variety of cross-sectional structures or profiles. For example, some cross-sectional profiles of various embodiments of elongate cores are shown in FIGS. 6A to 6K. The illustrated embodiments are not limiting, but merely examples of various possible cross-sectional profiles of any of the embodiments of elongate cores or orthopedic devices described herein. The illustrated embodiments shows a variety of possible cross-sectional shapes for embodiments of the device or the core of the device, including a square, ellipse, triangle, etc., and wherein the elongate core can be modified by twisting, and zig-zagging, and/or undergo one or more surface treatments such as abrading or pitting, for example.

FIG. 6A illustrates a cross-sectional view of an embodiment of a circular profile elongate core 640a, which can be rotated along a longitudinal axis of the core 640a. In various embodiments, the elongate core 640a is at least partially surrounded by an articular layer, wherein the elongate core 640a and/or the articular layer transition between a straight or slightly curved configuration to a more curved or arcuate configuration. During this change in configuration, elongate core 640a and the articular layer may rotate with respect to each other. In one embodiment, the elongate core 640a and the articular layer has some frictional engagement, which may interfere with rotation between the elements, resulting in some level of deformation. Furthermore, in one embodiment, both the elongate core 640a and the articular layer will have different material properties which are dependent on stiffness, durometer and other aspects of the respective materials. Depending on the desired orientation of an orthopedic device during delivery to a joint, the orientation of the elongate core 640a and/or the articular layer may be controlled by the configuration of the delivery device being used.

In certain embodiments, an elongate core may be configured with a non-circular cross-sectional shape. For example, FIGS. 6B to 6K illustrate cross-sectional views of a triangular profile elongate core 640b, a rectangular profile elongate core 640c, a trapezoidal profile elongate core 640d, an oval or elliptical profile elongate core 640e, a ridged profile elongate core 640f, a non-symmetric profile elongate core 640g, a cross or X-profile elongate core 640h, a lumen profile elongate core 640i, a pentagon profile elongate core 640j, and a hexagon profile elongate core 640k, respectively. In some embodiments, a non-circular profile may be used to resist or limit relative rotation or torsion of an articular layer and the core. Although several of the embodiments disclosed herein comprise one or more cores with an elongate configuration, in other embodiments, the cores may comprise a branching or interlinking structure that may have a generally planar or a generally non-planar structure. For example, some orthopedic devices may have a core with a “Y”-shape or “X”-shape branched configuration, with the arms or segments of the core arranged in a generally the same plane. Other orthopedic devices may also have a “Y”-shape or “X”-shape branched configuration but in a non-planar arranged, such as a three-leg or four-leg tripod arrangement, for example, where the intersection point of the “Y”-shape or “X”-shape is located in a different plane as one or more of the ends of the arms or segments. Embodiments of orthopedic devices having branched cores may or may not have articular layers are also branched, and embodiments of orthopedic devices with branched articular layers may or may not have branched cores.

In some embodiments, the articular layer of the orthopedic device may also comprise a non-circular cross-sectional shape. The cross-sectional shape of elongate core of such orthopedic devices, if any, need not have the same or similar the cross-sectional shape of the articular layer. In FIGS. 6L and 6M, for example, the orthopedic device 642L comprises an articular layer 644L with a rectangular axial cross-sectional shape and an elongate core 640L with a circular axial cross-sectional shape. The larger dimension 646L, if any, of the rectangular articular layer 644L may be generally oriented within the plane 648L of the orthopedic device 642L, while the shorter dimension 650L, if any, (or a dimension transverse to the larger dimension 646L) may be generally oriented transverse to the plane 648L of the orthopedic device 642L. In other embodiments, the orientation may be opposite, or may be at any other angle or orientation with respect to the plane of the orthopedic device, if any, or other geometric reference of the device, including but not limited to the longitudinal axis or a center axis of the orthopedic device, if any. The core 640L of the orthopedic device 640L may be generally centered along the larger dimension 646L and the shorter dimension 648L, i.e. at a position about 50% along the larger dimension 646L and the shorter dimension 648L. In other embodiments, the relative position of the core 640L may be located anywhere from about 0% to about 100% along a particular dimension, including about 10%, about 20%, about 30%, about 40%, about 60%, about 70%, about 80% and about 90%, for example. The relative position of the core may be generally uniform throughout the orthopedic device, or may vary depending upon the particular section of the orthopedic device. In further embodiments, where a portion of the core extends beyond an inner or bottom surface, or an outer or upper surface of the articular layer with respect to a particular dimension, the relative position may be expressed as a negative percentage or a percentage greater than 100%. In some embodiments, for example, the position of the core may be located at about −10%, about −20%, about −30%, about −40% or about −50% or lower, or about 110%, about 120%, about 130%, about 140% or about 150% or greater. The orthopedic device in FIG. 6L also illustrates that the C-shape or arcuate configurations described herein are not limited to generally circular devices, and may include generally oval devices.

FIGS. 6N and 6O depict another embodiment of an orthopedic device 642N, comprising an articular layer 644N with an “X”-shape cross-sectional shape along with a circular core 640N. FIG. 6P depicts another embodiment of an orthopedic device 642P, comprising a triangular articular layer 644P and a circular core 640P. In contrast to orthopedic devices 642L and 642N in FIGS. 6L and 6N, respectively, which depict open configurations, FIG. 6P illustrates an orthopedic device 642P with a closed configuration, as well as a cross-sectional shape that varies from one section 652P to another section 654P. Both features, however, need not be found in the same orthopedic device. In this particular example, one section 652P comprises an isosceles triangular shape while the other section 654P comprises an equilateral triangular shape. The different shapes of two or more sections of an orthopedic device, if any, may share one or more shape features (e.g., both may be triangular or polygonal), but in other embodiments, may be completely different (e.g. one section may have a small circular shape, while another section may have a large irregular octagonal shape).

FIG. 6R depicts still another embodiment of an orthopedic device 642R, comprising an articular layer 644R that has a non-polygonal cross-sectional shape that is non-uniform, along with a non-circular core 640R. As shown in FIG. 6S, one section 652R of the articular layer 644R comprises a superior protruding edge 656R and an inferior protruding edge 658R, both of which have a reduced profile in other section 654R of the device 642R. Furthermore, the inner protrusion 660R of one section 652R may also have a different profile compared to another section 654R. Still another feature of the device 642R is the presence of a second core 662R within the articular layer 644R. In this particular embodiment, unlike the primary core 640R, the second core 662R may be located in only a portion of the device 642R, such as the inferior protruding edge 656R, and may not extend along the entire circumference or perimeter of the device 644R.

FIGS. 6T and 6U depict another embodiment of an orthopedic device 642T, comprising a core 640T, an articular layer 644T and a span member 674T that crosses at least a portion of the inner region 676T of the orthopedic device 642T. In this particular embodiment, the span member 674T comprises a membrane having a generally uniform thickness and a planar configuration located generally midway between the superior surface 678T and the inferior surface 680T of the orthopedic device 640T. In other embodiments, the span member have a variable thickness, including one or more openings, depressions or grooves along one or more surfaces of the span member. In addition to planar configurations, the span member may have one or more regions with a non-planar configuration, including corrugated, concave, or convex regions, for example. The span member 674T may comprise the same or different material as the articular layer 644T, and may or may not be attached or embedded with reinforcement structures, e.g. wires, struts or meshes.

FIGS. 6V and 6W depict one example of an orthopedic device 642V with a span member 674V comprising a membrane structure with a convex configuration with respect to the superior surface 678V of the orthopedic device 642V. The span member 674V further comprises one or more through openings 682V arranged in a grid-like order, and with a generally cylindrical shape on cross-section, as shown in FIG. 6W. In other embodiments, one or more openings may have a non-circular shape (e.g. elliptical, ovoid, squared, rectangular, trapezoidal, or polygonal), have a non-uniform shape or diameter (e.g. tapered, toroidal), have a non-linear elongate configuration (e.g. angled or undulating), or any combination thereof.

FIG. 6X depicts another embodiment wherein a plurality of span members 674X are provided across the inner region 676X of the orthopedic device 642X. As shown in FIG. 6X, the span members 674X has an elongate configuration with a generally parallel orientation with respect to one another. In other embodiments, however, one or more span members may have a non-parallel or overlapping configuration with respect to another span member. Each of the span members 674X may be symmetrically oriented with respect to a midline through the orthopedic device, but may also be asymmetrically oriented. The span members 674X in FIG. 6X have any of a variety of cross-sectional shapes (e.g. circular, elliptical, ovoid, squared, rectangular, trapezoidal, or polygonal), and may have uniform or non-uniform cross-sectional areas or shapes along their elongate length.

As mentioned previously, the articular layer of the orthopedic device may comprise a smooth outer surface, or a porous or textured surface. In FIG. 6Y, for example, the articular layer 644Y of the orthopedic device 642Y comprises a textured surface with series of ridges having a repeating angular or oscillating pattern. As mentioned previously, in other embodiments, the textured surface may comprise other types of surface structures, including but not limited to discrete or aggregated microstructures or nanostructures, such as grooves, pores, indentations, hooks, barbs, tubes, rods, cones, spheres, cylinders, loops, pyramids, or a combination thereof. The surface of the orthopedic device or its articular layer may be completely or partially covered with the surface textures, and the density, spacing or size of the ridges or other surface structures may be uniform or non-uniform. In the embodiment depicted in FIG. 6Y, the ridges generally have the same orientation regardless of the particular section of the articular layer 644Y, but in other embodiments, the ridges, structures or textures may be aligned or oriented in any of a variety of other ways, including but not limited to with respect to the longitudinal axis of the orthopedic device 644P, or circumferentially around the device 644Y, for example.

In some embodiments, larger structures may be provided on the surface of the orthopedic device, in addition or in lieu of surface texturing. In FIG. 6Z, for example, an orthopedic device 644Z comprises a C-shape configuration with one or more ridges or flanges 664Z having a size that alters a gross dimension of the orthopedic device 644Z by about 5% or more. In this specific example, the flanges 664Z have a circumferential configuration around the body of the orthopedic device 644Z and are angled such that the narrow end 666Z of the flange 664Z is closer to the middle portion 668Z of the orthopedic device 644Z while the wider end 670Z of a flange 664Z is closer to the ends 672Z of the orthopedic device 644Z. In other embodiments, the larger structures may comprise large grooves or indentations, or other types of projecting surface structures. These larger structures may be rigid, semi-rigid or flexible, and each structure can have the same or a different configuration, size or material composition. In some embodiments, the flanges 664Z may resist migration or displacement of the orthopedic device 644Z within the joint space and/or out of the joint capsule.

In one embodiment, the articular layer can be at least partially attached to the outer surface of a portion of a backbone or core, either during or after implantation. In one non-limiting example, a core or backbone or wire of fixed length is implanted in a joint, then an articular layer or jacket is advanced over the core. In alternative embodiments, the articular layer is positioned in the joint first, followed by the insertion of the core through the articular layer. The core or backbone or wire is cut to size for a joint and is implanted in a joint, then an articular layer or jacket is advanced over the core. The articular layer or jacket may also be shaped or sized before being advanced over the core. In various embodiments, the core could have a feature such as a ball or hook at one or both ends (proximal and distal) so that when the articular layer is advanced over the proximal end of the core, the articular layer can abut against a distal feature or stop. In still other embodiments, the core may comprise a roughened outer surface, barbs, or other interference structures that resist separation from the articular layer. In an embodiment with a proximal feature such as a ball or cap, the articular layer may be trapped or held in position between the features to resist separation from the core. In other embodiments, heat bonding or adhesives may be used to attach the articular layer to the core. In one embodiment the articular layer can be implanted without a backbone or core.

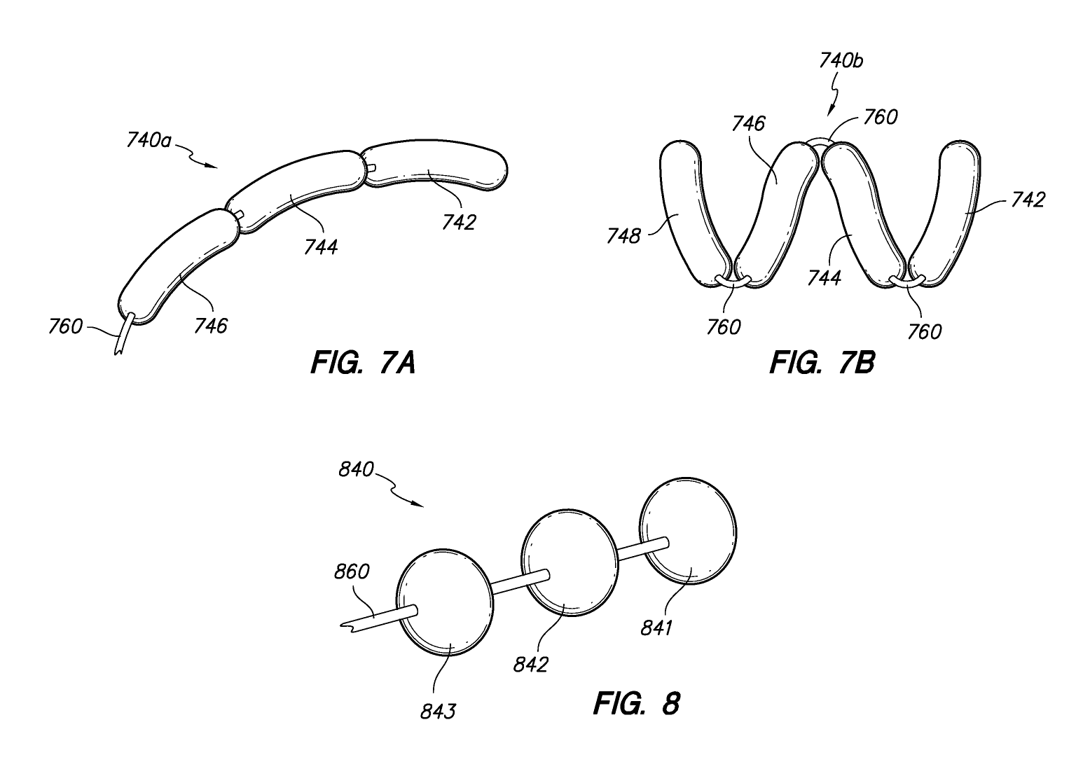

Some embodiments of an elongate core include a plurality of inter-connectable discrete elongate members, as shown in FIGS. 7 to 9C. In various embodiments, two or more discrete articular structures or members may be connected along a single core wire or a plurality of core wires or elements. In embodiments comprising a plurality of core elements, a separate core element may be used to connect each adjacent pair of articular members, or multiple core elements may be used. In other embodiments, one or more discrete articular members are configured to facilitate or permit rotation or spinning about the connector or core wire. In another embodiment one or more discrete elongate members are affixed to the connectors or core wire in a manner to reduce or prevent rotation of the elongate members with respect to connector or core wire. For example, multiple core wires may be beneficial in resisting rotation of an articular structure around a single core element. As illustrated in FIG. 7A, one embodiment of an orthopedic device 740a comprising a plurality of inter-connectable discrete elongate members 742, 744 and 746 which are linked by connector 760. In some embodiments, the connector 760 can be a single core member extending between all the discrete elongate members 742, 744 and 746, or it can be any number of discrete connecting members between the elongate members. In one embodiment, the connector is flexible or malleable such that orthopedic device can be arranged in a variety of non-linear configurations. In FIG. 7B, for example the orthopedic device may be manipulated to orthopedic device 740b with a plurality of independent or inter-connectable discrete elongate members 742, 744, 746 and 748 can having a “W”-shape generally rectilinear configuration. The connectors 760 can be configured to orient the elongate members such as 742, 744, 746 and 748 in any number of orientations or angles, in or out of plane. In some embodiments, the connectors 760 can have shape memory configurations or biases for particular orientations, depending on the doctor's preference or the device selected. The overall shape of an orthopedic device may comprise a “C”, “O” and “W”-shape, but the device and/or articular layer and/or elongate core can specific any shape or configuration or general class of shape or configuration as mentioned elsewhere herein. FIG. 8 illustrates an alternate embodiment where the orthopedic device 840 comprises non-elongate interconnected articular members 841, 842, and 843 which are linked by a connector 860 passing through each member 841, 842 and 843, for example. The articular members, may have any of a variety of other shapes and configurations, and need not have a uniform size and shape, or comprise the same material.

In some embodiments, the orthopedic device may be marked to indicate orientation of the device. For example, the orthopedic device can be marked with any of a variety of graphical or other detectable indicia, including but not limited to a symbol, text, colors, magnetic radiographic markers or inks, or other types of markings that can be sensed visually or otherwise with or without the assistance of sensors or other devices, to indicate a side or feature that should be directed to a specific location. In some embodiments, identifying the orientation of an orthopedic device when it is deformed to a substantially straightened configuration may be addressed by markings or other indicia on the device to provide an indication of the orientation of the device. The indicia can be helpful for checking proper function or delivery of the orthopedic device. In some embodiments, the device or a component thereof may comprise a material that has electroresistive property which may change when the device or component is stressed or deformed. Changes in these or other electrical properties may be used as assess the forces acting on the device.

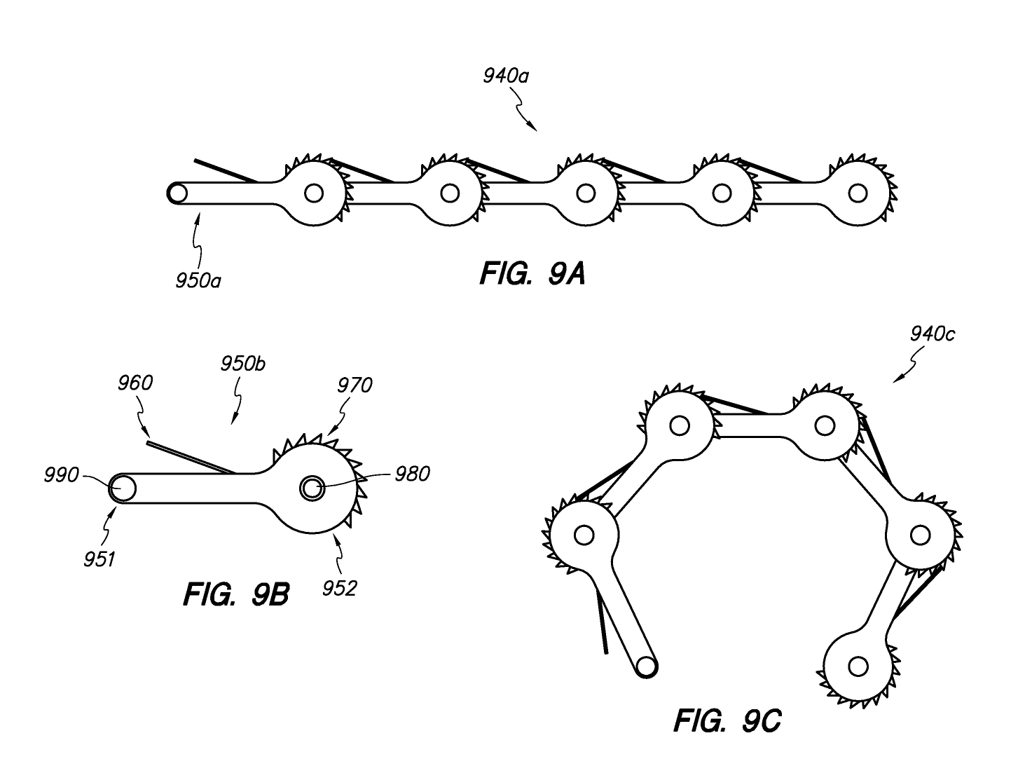

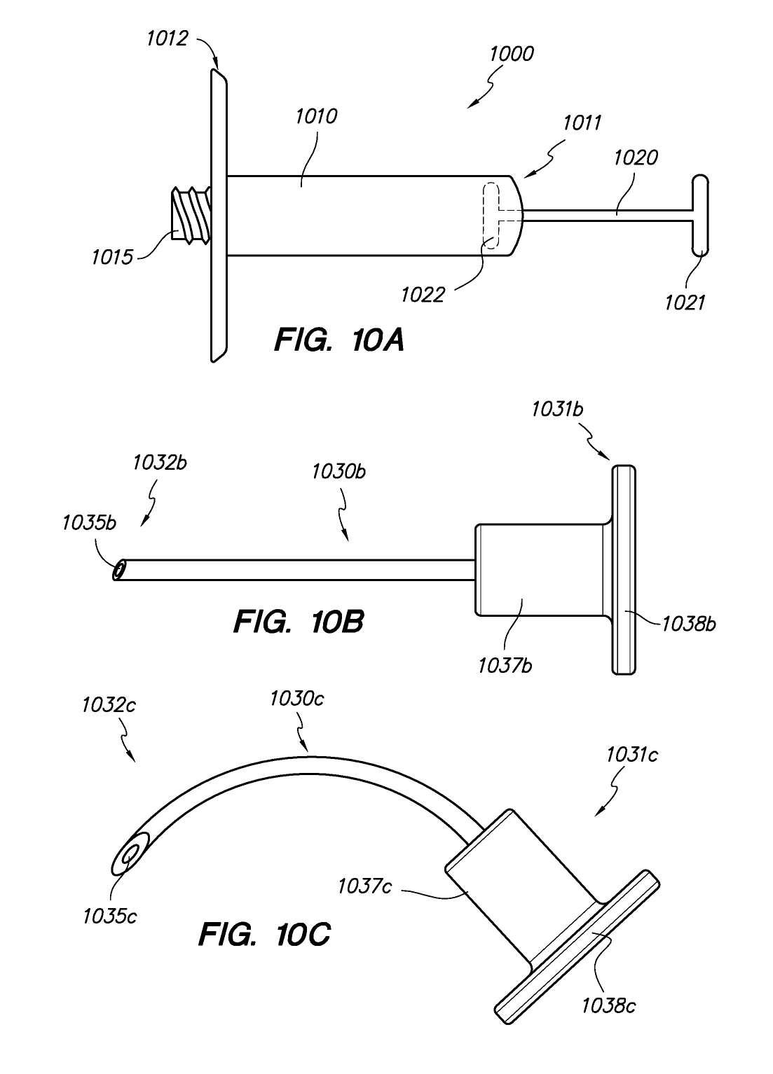

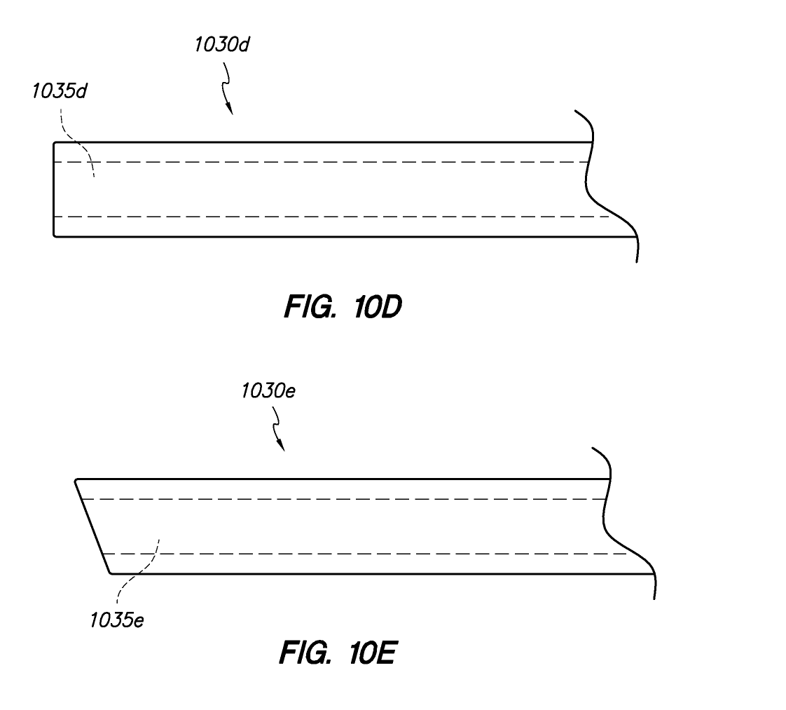

In some embodiments, the orthopedic device may comprise one or more articulations to facilitate configuration changes, in addition or in lieu of flexible interconnecting structures and/or materials. In one embodiment, for example, an elongate core 940a may comprise a plurality of interconnectable discrete members, or links 950a, in a substantially straightened configuration, as shown in FIG. 9A. The elongate core 940a may be described as a multi-link elongate core, multi-link core, multi-link orthopedic device, or multi-link orthopedic implant. The multi-link orthopedic device may comprise a series of rigid or flexible links configured to translate the multi-link core from a straight or slightly curved configuration into a curved orientation or configuration. The diameter of curvature of the device could be adjustable by the ratcheting features provided on each link 950a. In one embodiment the links 950a are made of a material that can undergo some level of elastic deformation. In another embodiment, the links 950a are made of a more rigid material. With embodiments of the device, core, or link that are made from a superelastic material such as Nitinol, the implant can be straightened from its curved, deployed or implanted configuration and placed in a needle or cannula. Using a curved delivery system, such as one shown in FIG. 10C below, would allow a more-rigid arcuate implant to be slightly straightened enough for insertion, but not enough to cause yielding.

FIG. 9B shows a side view of one link 950b. In one embodiment, link 950b is a link 950a of FIG. 9A. In one embodiment link 950b comprises a first end 951 and a second end 952. Various links 950b are inter-connectable between the second end 952 of a first link 950b and the first end 951 of a second link 950b′, and in one embodiment the interconnection is a hinged connection between a first link interface 990 and a second link interface 980. In other embodiments, other connections or joints may be used, such as a ball-and-socket joint, a pivot joint, or a saddle joint, for example. Each link connection need not be the same type of connection. In one embodiment, the first link interface 990 is a post and the second link interface 980 is a channel in which the post is captured to allow rotation. In another embodiment, the second link interface 980 is a post and the first link interface 990 is a channel in which the post is captured to allow rotation. In various other embodiments, other link interfaces allowing some rotation including snap fits, connectors, or other similar interfaces may be used. In the illustrated embodiment, the link 950b comprises a ratchet prong 960 and ratchet teeth 970. The ratchet teeth 970 of one link 950b interact with the ratchet prong 960 of a second link 950b′ to allow rotation with respect to links 950b and 950b′ while restricting or limiting rotation in the opposite direction.

Various link embodiments can be configured to an arcuate configuration, as in FIG. 9C, which shows an elongate core 940c with links in an arcuate open loop configuration. In one embodiment, the elongate core 940c is actuated and locked into an arcuate configuration by the ratcheting mechanism as described above. In one embodiment the ratchet locking is configured to be disengageable such that the prong is releasable from the teeth to allow the elongate core 940c to rotate in a straight or less-curved configuration.

The orthopedic devices described herein may be implanted using any of a variety of implantation procedures. Although certain embodiments are configured for minimally invasive implantation, surgical implantation using an open procedure is also contemplated. The orthopedic device described herein are may be implanted or be adapted for implantation into a variety of joints, including but not limited to the DIP and PIP joints of the hands and feet, the metatarsal-phalangeal joints, the tarsal-metatarsal joints, the metacarpal-phalangeal joints, the carpal-metacarpal joints, the ankle joints, the knee joints, the hip joints, the facet joints of the spine, the glenohumeral joint, the elbow joint, the temporomandibular joint and others.

In various embodiments of orthopedic devices described herein, the orthopedic devices are configured to have an arcuate shape in a joint. In certain embodiments, the orthopedic device can be straightened into a substantially straightened or less-curved configuration for implantation with an orthopedic device delivery system. For example, in one embodiment an arcuate orthopedic device can be straightened by cooling or chilling a shape-memory material in the orthopedic device and then inserting the orthopedic device into a tube, cannula, or hypodermic needle of specific design shape and cross section. The pre-loaded hypodermic needle is then attached to a handle through a coupling or interface such as a luer lock standard to the industry or any other attachment means. The physician then straightens the finger by applying force providing for a space or gap to occur in the joint. For example, the force can be provided by using his hands or a tool, to pull, stretch or spread the desired joint. In one embodiment, a sharp tool, such as a scalpel or trocar, can be used to pierce the joint tissue. In another embodiment, the delivery device needle can pierce the joint tissue. The needle is positioned mid-point between the posterior and anterior surfaces of the joint. The tip of the needle may be advanced into the joint within the joint capsule. Once inserted, the physician releases the device by advancing it out of the needle using an advancing mechanism, such as a handle and plunger. As used herein, a “plunger” may also be called a push rod, an advance rod, or an advance mechanism. Once deployed, the needle and handle may be removed from the joint. If more than one joint, such as a DIP, PIP or MCP joint, is treated, the deployed needle may be removed via the luer type connector and a second needle may be attached to the same handle, to permit repeating the procedure as needed.

One orthopedic device delivery system 1000, comprising a handle 1010 and a plunger 1020, that is suitable for delivering the orthopedic device implant is shown in FIG. 10A. In various embodiments, the orthopedic device delivery system 1000 can be provided in a number of mechanical configurations. In some examples, the orthopedic device delivery system 1000 may be used to completely advance the orthopedic device out of a channel, cannula, lumen, or needle, with non-limiting examples illustrated in FIGS. 10B and 10C. In various embodiments, the orthopedic device delivery system 1000 may be actuated by advancing the orthopedic device by a simple ram-type piston or hypodermic needle configuration, through the use of a lead screw, or through the use of a pneumatic or hydraulic type mechanism. In the illustrated embodiment, the handle 1010 comprises a distal handle region 1012 and a proximal handle region 1011, and the plunger 1020 comprises a distal plunger region 1022 and a proximal plunger region 1021. In one embodiment the distal handle region 1012 comprises a cannula interface 1015, such as a luer connector.

Embodiments of a cannula or needle can be straight or curved, as illustrated in FIGS. 10B and 10C respectively. A substantially straight cannula 1030b or needle with a lumen 1035b may be suitable for delivering the orthopedic device implant described herein in conjunction with the orthopedic device delivery system 1000 of FIG. 10A, for example. In one embodiment, the cannula 1030b comprises a distal cannula region 1032b and a proximal cannula region 1031b. The delivery cannula 1030b may be attached to a handle 1010 in an orthopedic device delivery system, such as orthopedic device delivery system 1000, with any of a number of attachment structures or assemblies, including but not limited to a standard luer type coupler, a bayonet, a luer mount, or a thread mechanism for attachment to the delivery handle 1010. In one embodiment, proximal cannula region 1031b comprises a flange 1038b and a luer connector 1037b. The needle or deployment cannula 1030b may be provided in many shapes and cross sections. In one embodiment, the cannula 1030b is sized and configured to interface with the orthopedic device in a specific orientation for delivery into a joint. This interface may be a key-slot, or other type of mechanical interface. In one embodiment, the distal cannula region 1032b may be provided at its distal end with an insertion feature such as a point, knife edge or blunt atraumatic edge. Another embodiment of orthopedic device delivery system comprising an arcuate cannula 1030c or curved needle is shown in FIG. 10C. It may have a lumen 1035c configured to deliver an orthopedic device implant described herein in conjunction with the orthopedic device delivery system 1000 of FIG. 10A, for example. In various embodiments, an arcuate cannula 1030c is similar to substantially straight cannula 1030b, except that arcuate cannula 1030c is more curved.

In some embodiments, the process or method of inserting an orthopedic device into a joint is preferably atraumatic. For example, a fluoroscopically placed stab incision may be followed by a cannula insertion for orthopedic device delivery. The stab incision may provide a path for a delivery needle or cannula to follow. The stab incision may also permit the cannula tip to be non-sharpened. For example, a joint such as a DIP, PIP or MCP joint can be physically identified for orthopedic device placement. The device can be fluoroscopically placed or inserted without fluoroscopy, then a cannula is inserted into the stab incision and the orthopedic device is delivered through the cannula in the incision to the joint.

Referring to the tip of a needle or cannula, FIGS. 10D and 10E illustrate two variant embodiments. A blunted delivery cannula 1030d with a lumen 1035d is shown in FIG. 10D. In certain embodiments, the blunted delivery cannula 1030d may be used in conjunction with a joint piercing tool (not illustrated here) such as a knife, scalpel, spike, trocar, or other sharp instrument for piercing tissue surrounding a joint in order and to create an access hole or port through which the orthopedic device can access the joint. An angular tip 1030e with a lumen 1035e is shown in FIG. 10E. In one embodiment, the angular tip 1030e is sharp enough to pierce tissue surrounding a joint in order to create an access hole or port through which the orthopedic device can access a joint. In another embodiment, the angular tip 1030e is atraumatic and may be used to guide the delivery device in a previously opened incision or natural opening in tissue. Minimally or atraumatic distal cannula regions 1032b, 1032c corresponding to any cannula, such as cannulas 1030b to 1030e are intended to be slid through the stab incision, such as made by a scalpel, thereby spreading the tissue which makes up the knuckle capsule as it goes in.

As described above, in various embodiments an elongate core is at least partially surrounded by an articular layer, wherein the elongate core and/or the articular layer actuate between a straight or slightly curved configuration to a more curved or arcuate configuration. During this change in configuration, elongate core and the articular layer may rotate with respect to each other. In one embodiment, the elongate core and the articular layer may have some frictional engagement, which may interfere with rotation between the elements, resulting in some level of deformation. Furthermore, both the elongate core and the articular layer may have different material properties which are dependent on stiffness, durometer and other aspects of the respective materials. Depending on the desired orientation of an orthopedic device during delivery to a joint, the orientation of the elongate core and/or the articular layer may be controlled by the configuration of the delivery device being used. In various embodiments, the shape, curvature, or tip of the cannula, needle, or lumen may be configured to control the specific orientation of the orthopedic device as it is being implanted. For instance, the point of a needle, trocar, or angle-tipped cannula such as an orthopedic device delivery system with an angular tip 1030e may be used to define the relationship of the orthopedic device and its orientation in a joint.

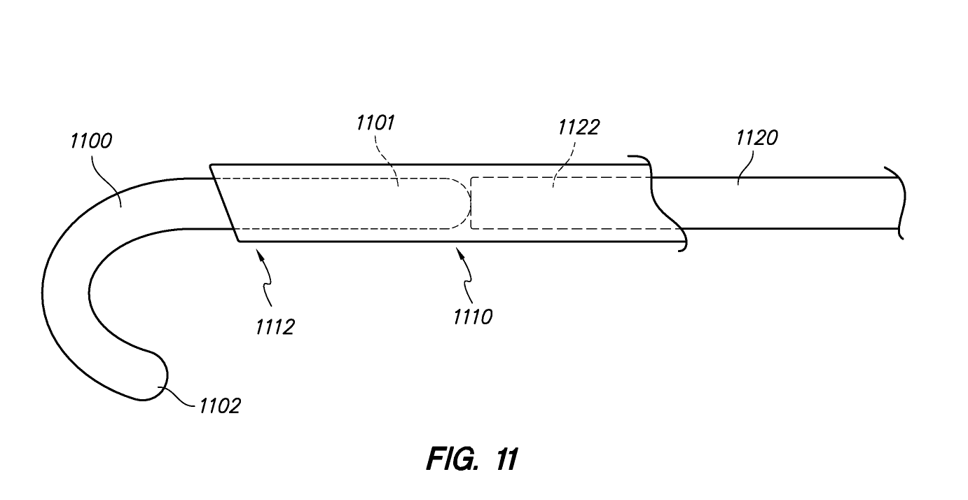

One embodiment for delivering the various orthopedic devices is shown in FIG. 11, where an implantable orthopedic device 1100 is advanced through a cannula 1110 by a plunger 1120. The orthopedic device 1100 comprises a distal end 1102 and a proximal end 1101, and may be similar to the embodiments of orthopedic devices described herein. The cannula 1110 may have a distal end 1112 that is configured to present the orthopedic device 1100 at the implant delivery site in a joint in the proper orientation. The plunger 1120 has a distal end 1122 which advances the orthopedic device 1100 out of the cannula 1110 and into the joint. In the illustrated embodiment, the distal end 1122 of the plunger 1120 may be used to push the proximal end 1101 of the orthopedic device 1100. In one embodiment, the plunger may be sized to match the cross-sectional diameter of the proximal end of the device and may also be provided with features to engage the device in a specific fashion. In other embodiments, the plunger may be configured to attach to a distal or medial portion of the orthopedic device and to pull or advance the device out of the cannula. In one embodiment, an orthopedic device delivery system is configured to deliver a multi-planar orthopedic device from a point corresponding to the distal tip of a cannula into joint. In one embodiment, the orthopedic device delivery system may be configured to deliver the orthopedic device 1100 in an orientation within a plane (“primary plane”) that may generally correspond to a plane of bony or cartilaginous articulation within a joint that is generally orthogonal to a longitudinal axis of at least one bone comprising part of the joint. As an orthopedic device is delivered into a joint, such as a metacarpo-phalangeal joint, the tissue surrounding the joint, including a joint capsule and various ligaments and tendons, may help to maintain the orientation of the orthopedic device in or near the primary plane by containing the orthopedic device around its outer periphery. In one embodiment, an angular tip at the distal end 1112 of the cannula 1110 may help to maintain the proper orientation of the orthopedic device 1100 within or near the primary plane and to reduce or eliminate undesired bias or deformation of the orthopedic device 1100.

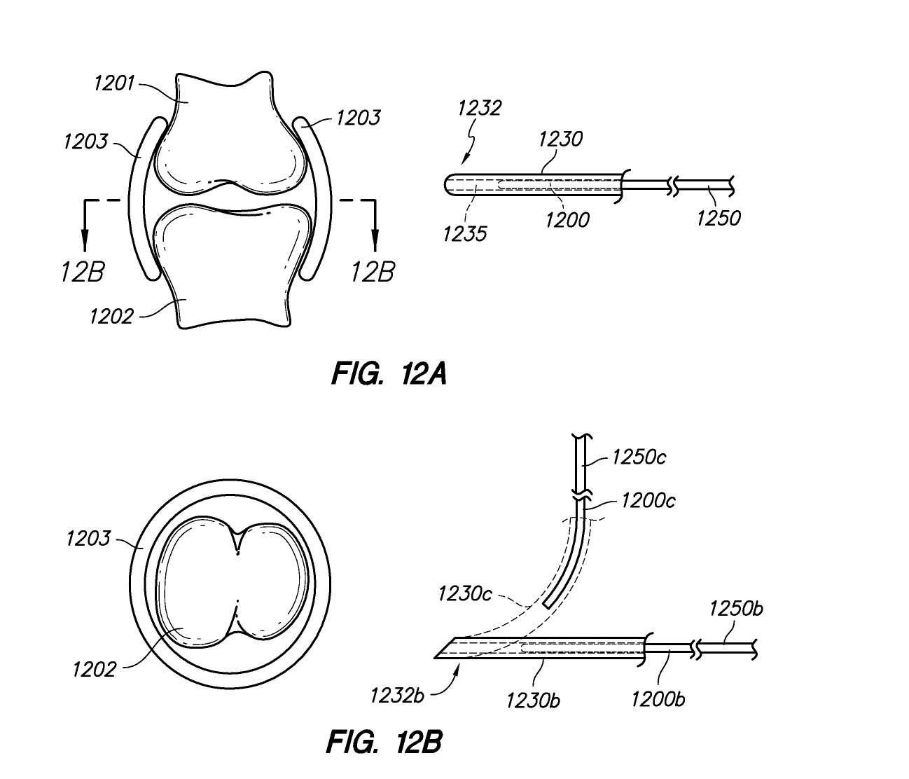

One method that may be used to deliver an orthopedic device 1200 to a joint using an orthopedic device delivery system is illustrated in FIGS. 12A to 15B. In these figures, a joint comprises a first bone 1201, a second bone 1202, and tissue 1203 surrounding the joint, such as a joint capsule and/or a ligament. The “A” figures illustrate a side view of the joint and the “B” figures illustrate a cross-sectional view orthogonal to the side view in “A.” The primary plane of the orthopedic device generally corresponds to the plane of the “B” when bones 1201 and 1202 are generally linear or aligned. When the bones 1201 and 1202 move with respect to each other, the primary plane may move as well, and may generally correspond to a plane normal to a point of contact between the bones 1201 and 1202 with the orthopedic device 1200. In one embodiment, the joint is a metacarpo-phalangeal joint, but in other embodiments, the joint may be an interphalangeal joint (e.g. DIP, PIP or IP joint), for example. In various embodiments, the point of insertion of a cannula into the joint can be anywhere along the periphery of the joint capsule of the joint, such as at a side, the top, or the bottom of the joint, which in one embodiment could correspond to the medial or lateral sides of a finger, the posterior surface of the finger (e.g. the dorsum of the hand) or the anterior surface of the finger (e.g. the palmar surface). A cannula 1230 with a distal end 1232 and a lumen 1235 is shown in both views. In the illustrated embodiment, the distal end 1232 of the cannula 1230 may comprise a feature or structure which helps maintain the proper orientation of the orthopedic device during delivery. As shown, in one embodiment, of the distal end 1232 comprises an angled tip. In each of FIGS. 12B, 13B, 14B and 15B, two embodiments of a cannula 1230b and 1230c are illustrated. While the cannulas 1230b and 1230c may be used one at a time, both cannulas 1230b and 1230c are illustrated (with cannula 1230b in solid lines and 1230c in dotted lines) to demonstrate that a straight or curved cannula, respectively, can be used to deliver the orthopedic device as described with respect to FIGS. 10B and 10C above. A plunger 1250 or other type of push member may be used to advance the orthopedic device 1200 into the joint using any of the advancing mechanisms described herein.

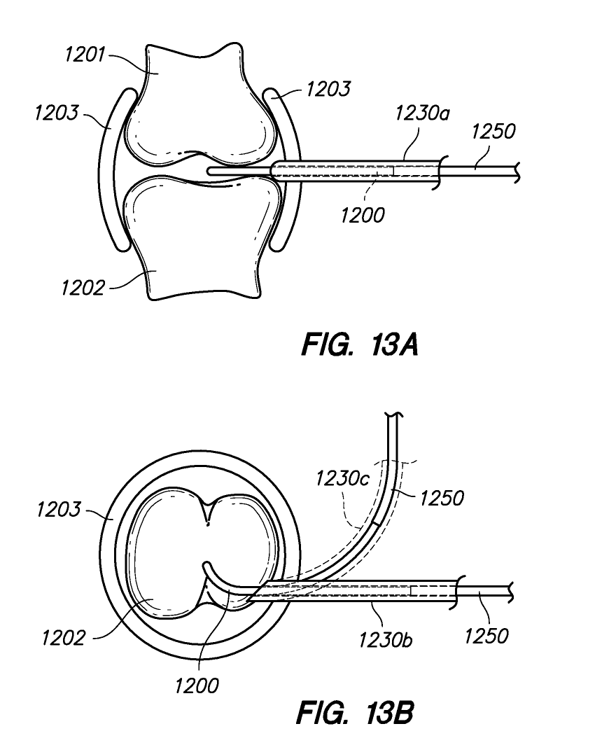

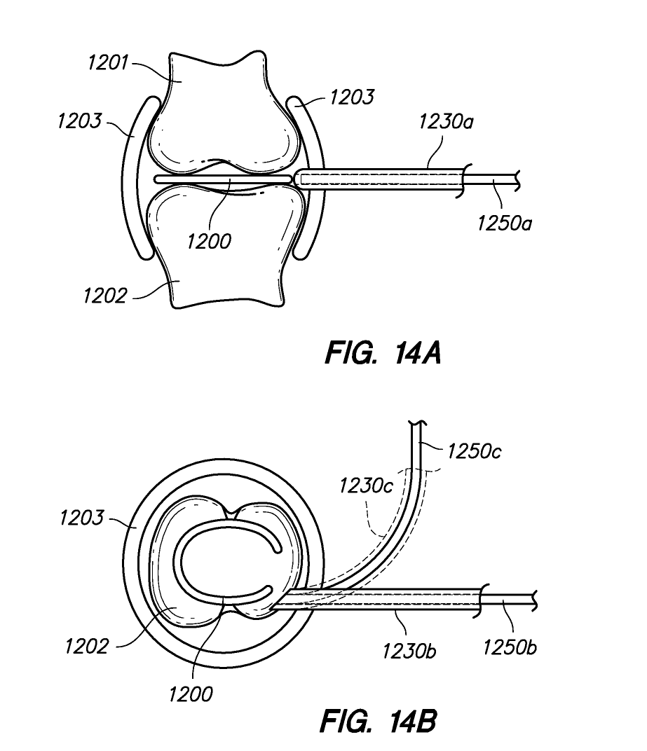

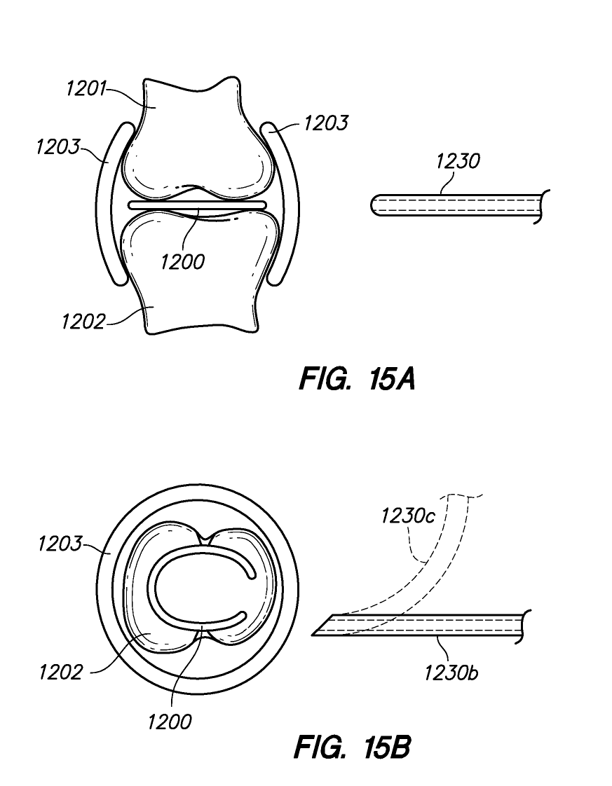

FIGS. 12A to 12B depict one embodiment of the delivery procedure prior to device implantation. Here, both a substantially straight cannula 1230b and another embodiment comprising an arcuate cannula 1230c are shown, while FIGS. 13A and 13B depict at least the partial insertion of the orthopedic device 1200 into the joint. In one embodiment a tool (not illustrated) may be used to pierce the tissue 1203 with a stab incision prior to insertion of the cannula 1230. In another embodiment, the cannula 1230 may be configured to directly pierce the tissue 1203. Once access into the joint is achieved, the plunger 1250 may be used to advance the orthopedic device 1200 into the joint, as shown in FIGS. 14A to 14B, with the orthopedic device 1200 in an arcuate configuration. The deployment of the orthopedic device 1200 into the joint and removal of the delivery cannula(s) 1230b or 1230c is illustrated in FIGS. 15A to 15B.

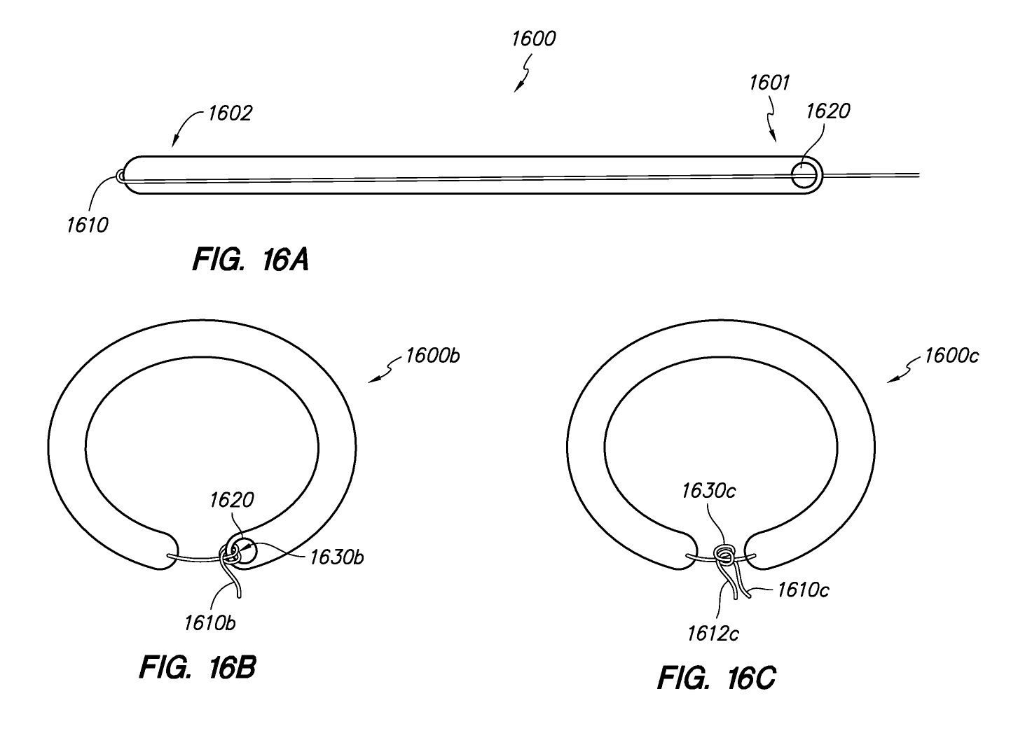

Other embodiments of orthopedic devices may have additional features which can be used to control the extent to which a device is open or closed. For example, one orthopedic device 1600 may comprise a tether 1610 and a loop structure 1620, shown in a substantially straightened configuration in FIG. 16A. The orthopedic device 1600 may exhibit similar characteristics as the previously described devices discussed herein. For example, the straightened configuration of the device 1600 may correspond to a configuration used for device delivery. In a deployed state, the device 1600 may have an open ring, arcuate, or other configuration or shape when it is not straightened for delivery or removal. The orthopedic device 1600 may comprise a proximal end 1601 and a distal end 1602. The distal end 1602 may be fixedly coupled to a suture or tether 1610 which is slidably coupled to an aperture or a loop structure 1620 located about the proximal end 1601. The tether 1610 may be a lanyard, suture, wire, or other structure which in one embodiment is unitary with the orthopedic device 1600. In one embodiment, the tether 1610 may be contiguous or integrally formed with an elongate core in the orthopedic device 1600. After the orthopedic device 1600 is deployed in a joint, it may assume an arcuate configuration as shown in FIG. 16B. In one embodiment, the tether 1610b may be pulled tight to bring the proximal end 1601 and distal end 1602 of the orthopedic device 1600 toward each other, and the tether 1610b may be optionally tied into a knot, plug, clip, clamp, mechanical fastener or other securing mechanism 1630b to form a substantially closed ring configuration for the orthopedic device 1600b. Depending on the degree of desired openness in the arcuate configuration of the orthopedic device 1600, the tether 1610b may be pulled and/or locked at different lengths to create a desired hoop or device size. Once the desired size is attained, the securing mechanism 1630b can be locked. The tether 1600b can then be cut proximate to the proximal side of the securing mechanism 1630b and removed from the joint. The tether 1600b may also be used for retrieval of a device that is improperly deployed in the joint, or for any other reason for removing the device. In some embodiments, for example, the tether may be manipulated to reposition an implant, to extract the implant, or it ma be cut and pulled out of the joint to pull the implant for retrieval of the device from the joint. In another embodiment, an orthopedic device 1600c comprises one or more tethers, such as tethers 1610c and 1612c as shown in FIG. 16C. The tethers 1610c and 1612c may also be secured to each other with a securing mechanism 1630c, as previously described with respect to securing mechanism 1630b. The tethers 1610c and 1612c can then be cut proximate to the proximal side of the securing mechanism 1630c. The tether 1610c and/or 1612c can also be used for repositioning or removal of the tethers or the tethers with the device from the joint, as described with tether 1600b.

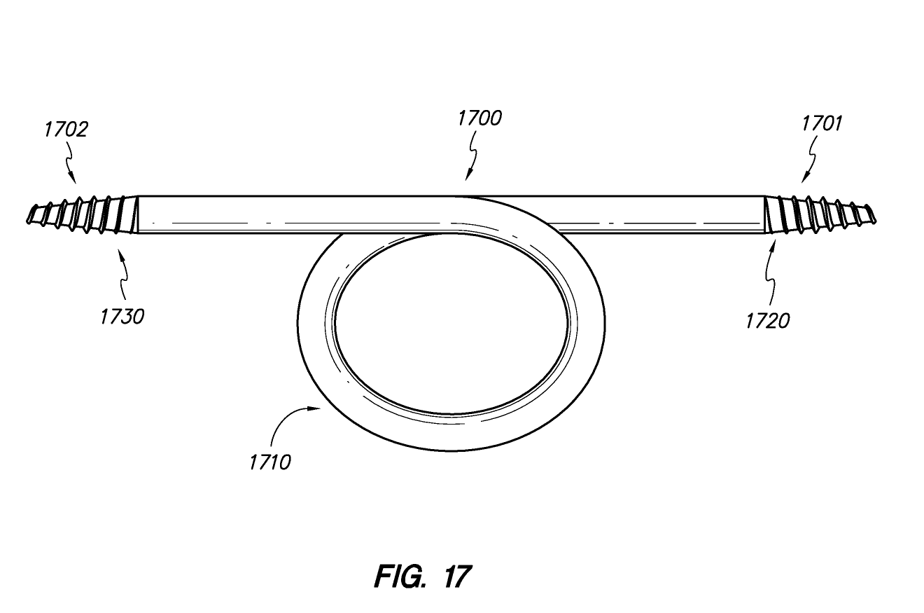

Another embodiment of an orthopedic device 1700 includes a looped arcuate configuration 1710 and at least one anchor, as is shown in FIG. 17. The orthopedic device 1700 may comprise a proximal end 1701 and a distal end 1702. In one embodiment, the orthopedic device 1700 may be configured so that the proximal end 1701 and distal end 1702 are crossing ends on substantially the same axis. In one embodiment, the orthopedic device 1700 optionally has a proximal anchor 1720 at the proximal end 1701 and/or a distal anchor 1730 the distal end 1702. In some embodiments, the orthopedic device 1700 has a substantially straight or less-curved configuration (not illustrated) for delivery. Once the orthopedic device 1700 is delivered to the joint, it may reconfigure toward its looped arcuate configuration 1710. As mentioned previously, in various embodiments, the anchors 1720 and 1730 may be contiguous or integrally formed with an elongate core of the orthopedic device 1700, the articular layer in the orthopedic device 1700, and/or are formed of separate elements and attached to the orthopedic device 1700. In various embodiments, the anchors 1720 and/or 1730 may have any of a variety of configurations, including threaded, tapered, cylindrical, barbed, hook-like, rib-like, and/or non-symmetric. One or more of the anchors 1720 and 1730 may also be dissolvable or drug eluting, for example. In one embodiment, the anchors 1720 and/or 1730 may be generally cylindrical and may be configured to be releasably attachable with a tool or plunger. In one embodiment, the anchors 1720 and/or 1730 are impregnated with a bonding material. The anchors 1720 and/or 1730 may be secured to any of a variety of tissue, including the tissue surrounding or in the joint, such as bone, cartilage, the joint capsule or the adjacent ligaments or tendons. In one embodiment, the anchors 1720 and/or 1730 may be bio-absorbable into surrounding tissue.

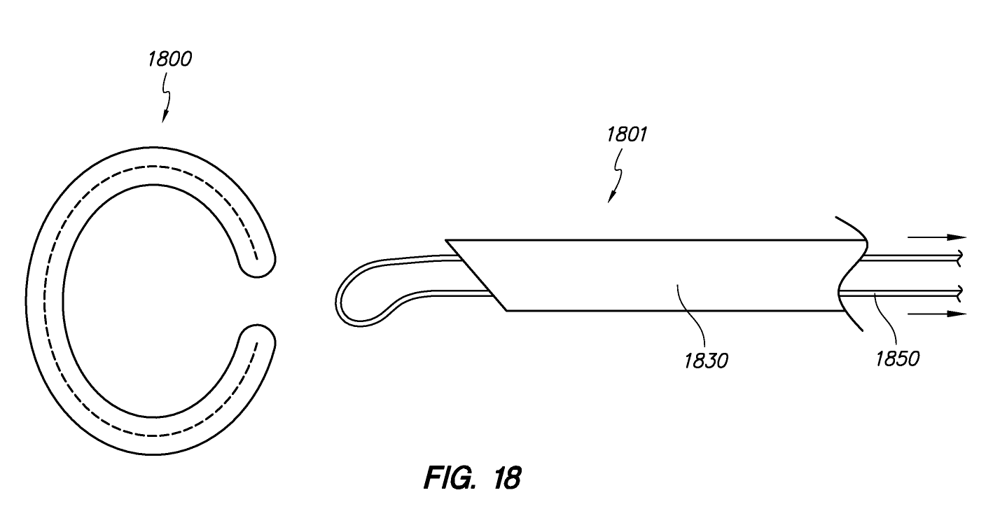

Retrieval of orthopedic devices is also contemplated. For example, one orthopedic device delivery and retrieval system 1801 may be configured to grab or couple to an implantable orthopedic device 1800 and pull it through a cannula 1830 using a snare 1850, as is illustrated in FIG. 18. The orthopedic device delivery and retrieval system 1801 may also be configured to deploy and/or retrieve the implantable orthopedic device 1800. In one embodiment, the cannula 1830 is part of a separate retrieval system with a lumen sufficiently sized and configured to recapture and retrieve a deployed orthopedic device 1800. In various embodiments, the orthopedic device 1800 may have end segments or medial segments along the orthopedic device 1800 articulate layer and/or elongate core, such as is illustrated in FIGS. 5A to 5C. In one embodiment, the orthopedic device 1800 may comprise one or more snare interface points, such as end segments 561a and 562a described with respect to FIGS. 5A and 5B above. For example, end segments 561a and 562a may comprise a ball, sphere, bead, hook, loop or other structure which may be ensnared by a tightened snare 1850 to pull the orthopedic device 1800 out of the joint. In one embodiment, the target structure of the orthopedic device 1800 is radiopaque or has markers for fluoroscopic visualization during the retrieval procedure. The snare 1850 may be attached (not illustrated) to a handle or control device proximal to the cannula 1830. For example, the snare 1850 may be attached to a handle or plunger with can then used to withdraw or pull with respect to the cannula 1830 to tighten the snare 1850 and to pull the orthopedic device 1800 out of the joint and out of the patient's body.

In one embodiment of an orthopedic device retrieval system 1801, the distal end of the cannula 1830 comprises a hook (not illustrated) which can be used to grab or retrieve an orthopedic device. The cannula hook may be actuatable by the doctor by pressing a button or other actuating mechanism to extend and/or rotate the hook into the joint, which then connects or grabs a part of the orthopedic device for retrieval. In an additional embodiment, the button or other actuating mechanism can be released to pull the hook back into place to lock on to the orthopedic device to be recaptured.

In one embodiment of an orthopedic device retrieval system 1801, only an elongate core is retrieved, leaving the articular layer in the joint in a manner similar to that discussed above regarding FIG. 2. Various embodiments of an orthopedic device with a removable core are disclosed in U.S. application Ser. No. ______, entitled “ORTHOPEDIC JOINT DEVICE WITH REMOVABLE CORE”, filed on ______, 2008, which is hereby incorporated by reference in its entirety.

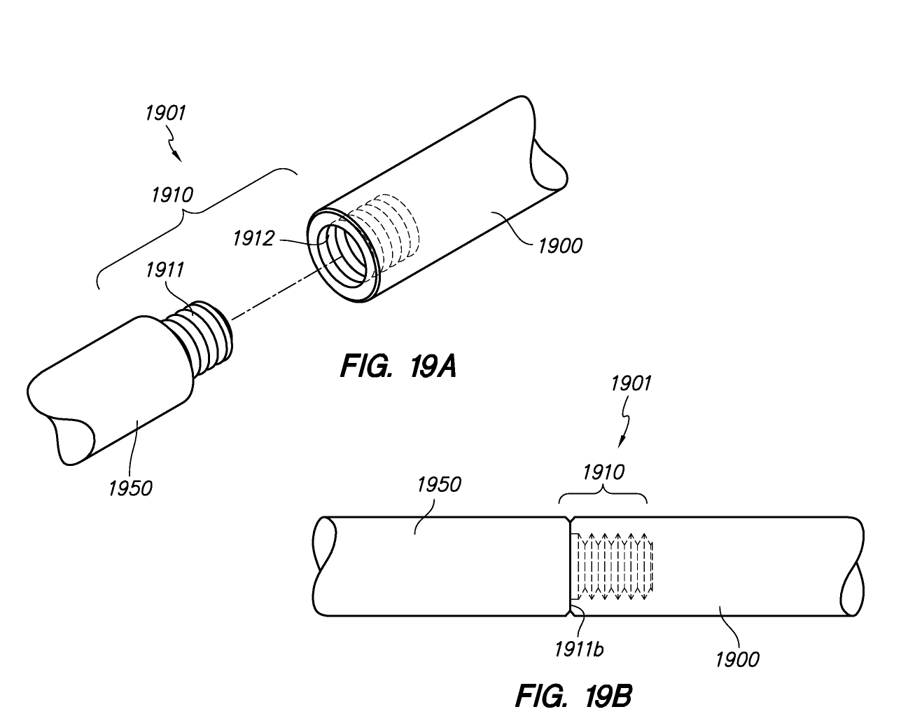

In another example, an orthopedic device retrieval system 1901 may be configured to retrieve an implantable orthopedic device 1900 with a plunger 1950 connectable with a device interface 1910, as shown in FIGS. 19A to 19B. Certain embodiments of devices connectable with device interfaces may allow for the final deployment and/or fine-tuning positioning or re-positioning of the orthopedic device once the orthopedic device is out of the cannula of the delivery system. In one embodiment, the device interface 1910 is a junction with a male threaded section 1911 on the distal end of the plunger 1950 and a female threaded section 1912 on the proximal end of the orthopedic device 1900. In other embodiments, the device interface 1910 may be a junction with a female threaded section 1912 on the distal end of the plunger 1950 and a male threaded section 1911 on the proximal end of the orthopedic device 1900. In one embodiment, the minor diameter of the threads of the male threaded section 1911 may generally be the same or correspond to the outer diameter of the plunger or orthopedic device. In one embodiment, the major diameter of the threads of the male threaded section 1911b may be less than the outer diameter of the plunger or orthopedic device which may provide uniform contact with the orthopedic device 1900.

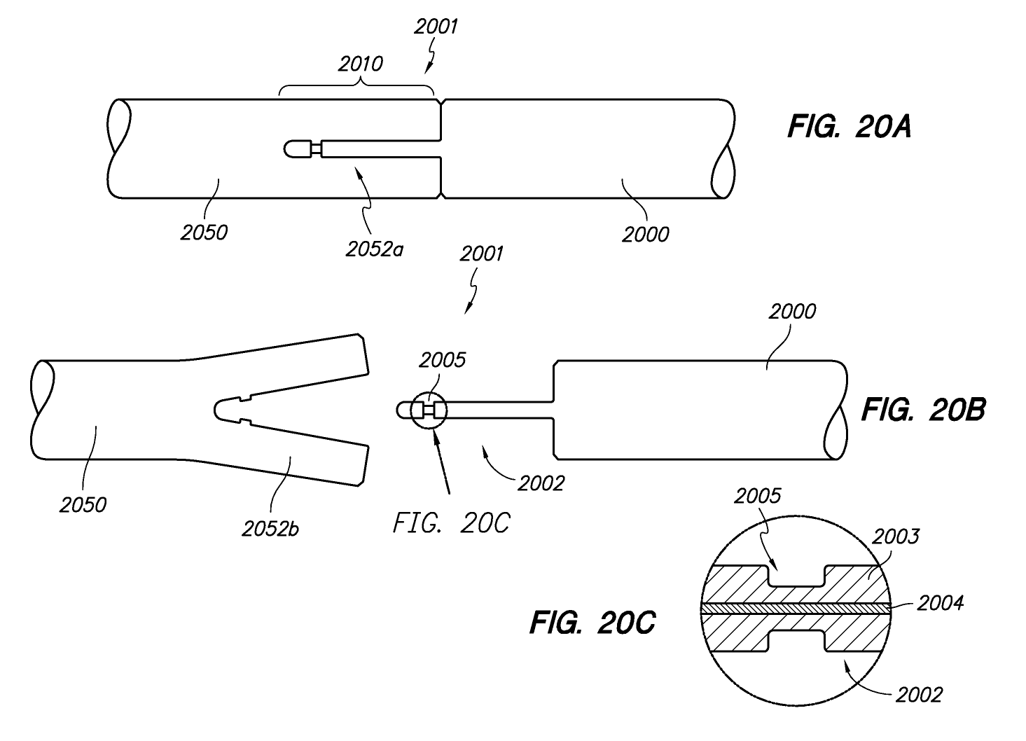

In still another example of an orthopedic device retrieval system, an implantable orthopedic device 2000 may be removed from its location using a plunger 2050 connectable with a device interface 2010, as is shown in FIGS. 20A to 20C. The device interface 2010 may be a junction with closed jaws 2052a at a distal end of the plunger 2050 and a jaw interface 2002 on the proximal end of the orthopedic device 2000. The jaw interface 2002 comprises a groove or recess 2005 which may facilitate grasping or locking of the jaw interface 2002. The groove or recess 2005 can be a linear, circumferential, or other feature for grasping with the jaws. In various embodiments, the jaw interface 2002 comprises a portion of an articular layer 2003, a portion of an elongate core 2004, or, as illustrated in FIG. 20C, both a portion of an articular layer 2003 and a portion of an elongate core 2004. In one embodiment, the jaw interface 2002 comprises a portion of the elongate core 2004, the elongate core 2004 is exposed at the jaw interface 2002. The closed jaws 2052a may be actuated to open jaws 2052b to release the orthopedic device 2000 into a joint. Conversely, the open jaws 2052b may be actuated to closed jaws 2052a to recapture the orthopedic device 2000 from the joint. In one embodiment, the jaws 2052a and 2052b may be spring loaded or otherwise biased to the open jaws 2052a configuration, the closed jaws 2052b configuration or a configuration therebetween. In alternative embodiments, the device interface 2010 may comprise a solenoid, linkage, ring mechanism, push-pin, snap-fit, and ball-detent interface. In one embodiment, the device interface 2010 may be an electrolyte junction whereby the application of energy, such as electricity, causes the junction to dissolve thereby breaking the junction between the plunger 2050 and the orthopedic device 2000.

In various embodiments, an orthopedic device delivery system may be configured to modify the shape or configuration of an orthopedic device between two, three, or more configurations. As shown generally in FIGS. 10A to 10C, one embodiment of the orthopedic device may comprise a needle pre-loaded with an orthopedic device, the orthopedic device may be held in a first configuration (such as a substantially straightened configuration) while stored in a delivery device and then actuated to deliver the orthopedic device in to a patient, where the device changes into a second configuration (such an arcuate or rectilinear configuration). In one embodiment, an orthopedic device delivery system can be configured to modify the shape or configuration of an orthopedic device between three or more configurations: a first configuration in which the orthopedic device is stored in the orthopedic device delivery system, a second intermediate configuration in which the orthopedic device is advanced through a lumen of a cannula in the orthopedic device delivery system in to a patient, and a third configuration in which the deployed orthopedic device is in its proper delivered orientation in the body of the patient. In one embodiment, the first and third configurations may be the same or similar configurations, wherein the first configuration is configured to reduce stress or strain on the orthopedic device while it is being stored by approximating, mimicking, or taking on the identical configuration of the third configuration as deployed in a patient's body to serve its function as an implant. For example, certain embodiments of delivery systems may contain a pre-loaded delivery device wherein an orthopedic device is held in or near its normal, non-straightened configuration, which in various embodiments may include curved, round, or rectilinear configurations that the would be found in the patient's body. The orthopedic delivery device can then be delivered in its proper orientation into the body. In some examples, retaining an orthopedic device in or near its normal, non-straightened configuration may reduce strain on the orthopedic device. In one embodiment, an orthopedic device may be removed from the body of patient by moving the orthopedic device from a deployed configuration in situ to a fourth configuration in a retrieval system. The fourth configuration may be the same or similar to an intermediate or storage system (corresponding to the second intermediate or first storage configurations described above).

Various embodiments of device loaders, loading device, or cassettes can be used to hold orthopedic devices in a first, non-straightened configuration while ensuring proper orientation for delivery of the orthopedic device to the body of the patient. In one embodiment, a loading device provides for minimally invasive delivery in a directed orientation to a joint of a patient. In one embodiment, the delivery orientation of an orthopedic device is provided by a loading device that orients the orthopedic device such that a grip on an orthopedic device delivery system holds the orthopedic devices in a second substantially straightened configuration in a lumen of a needle, catheter or cannula such that plane or orientation such that the orthopedic device exits a lumen into a joint in an orientation or plane that is substantially parallel to a plane between the articulating surfaces of the bone and/or cartilage in a joint. FIGS. 12A to 15B illustrates an embodiment of proper orientation of the delivery of an orthopedic device to a joint in a patient, wherein a plane for device delivery is represented in side view in FIGS. 12A, 13A, 14A and 15A corresponding to the plane of the drawing in FIGS. 12B, 13B, 14B and 15B.

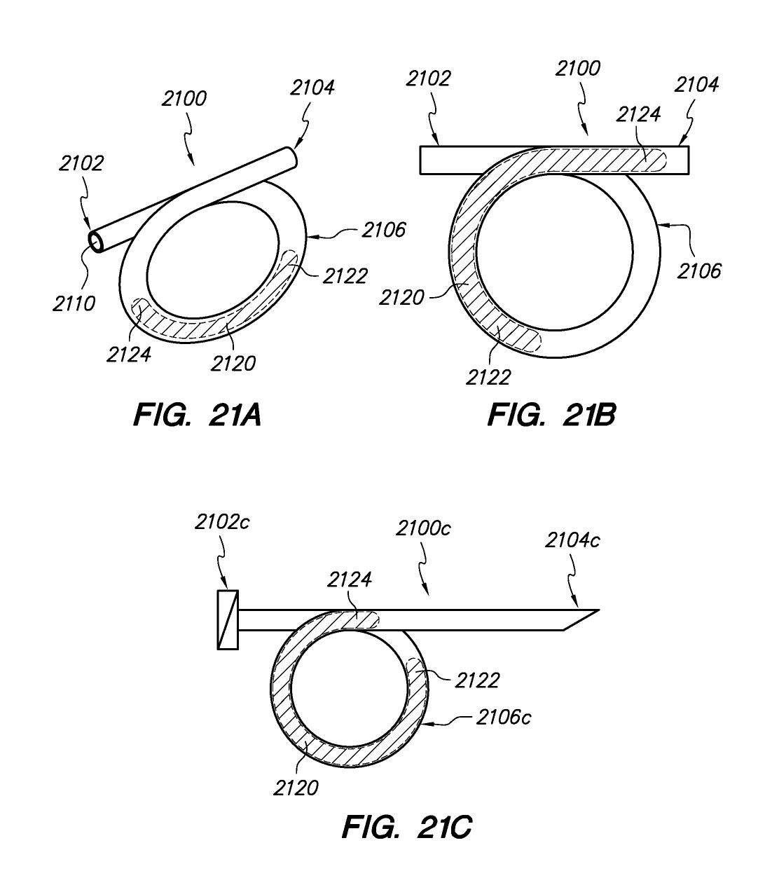

Various embodiments of an orthopedic device delivery system may comprise a loading device (also called a device loader or cassette described below in relation at least to FIGS. 21A to 28E and 31A to 40B) that holds one or more orthopedic devices in a non-straightened configuration until the orthopedic device is substantially straightened for delivery through the lumen of a cannula, catheter or needle into a delivery site in the patient's body. In various embodiments, loading devices may comprise a channel sized to be larger than an outer dimension of the orthopedic device. Although the various examples disclosed herein may reference a single orthopedic device, any of the loading device embodiments may be loaded or pre-loaded with one or more orthopedic devices. Some exemplary loading device may be loadable with an attachable/detachable cassette or loader clip or carrier in which the one or more orthopedic devices can be sequentially deployed using an advancement delivery and/or retrieval mechanism. In various embodiments, the loading device, cassette, loader clip, or carrier may be selectively attached to a delivery system.

FIGS. 21A and 21B illustrate one embodiment of a loading device 2100 comprising a proximal end 2102, a distal end 2104 and a loop 2106 with a channel 2110 (or lumen) extending there through. The loader 2100 may similar to a circular or rounded tube that in one embodiment has the proximal end 2102 and distal ends 2104 continue past one another after they have complete a 360 degree revolution. The channel 2110 may sized to be slightly larger than the outer dimension of the orthopedic device 2120 and may configured to allow the orthopedic device 2120 to be slidably advanced distally into the patient or a needle, or to be slidably retracted proximally. In various embodiments, one or both of the proximal end 2102 and distal ends 2104 may comprise an attachment interface, such as a connector. In one non-limiting example, the attachment interface can be a Luer connector, wherein the proximal end 2102 would connect to a deployment handle or similar structure, and the distal end 2104 would connect to a needle. For example, the proximal end 2102 Luer connector could be a female Luer configured to attach to a male Luer on a proximal device. The distal end 2104 Luer connector could be a male Luer connector configured to attach to a female Luer on a distal needle. In another embodiment, two or more loaders 2100 could be connected in series in order to deploy two or more orthopedic devices 2120. Advancement of the orthopedic devices 2120 through the one or more loaders 2100 may be accomplished using a flexible plunger (not illustrated) that is long enough to advance the orthopedic device 2120 out of the one or more loaders 2100. In various embodiments, a plunger (not illustrated here) may be used to move the orthopedic device from its base or native non-straightened configuration to a more straightened or slightly curved configuration in a needle for delivery to a joint.

FIG. 21C illustrates another embodiment of an orthopedic device delivery system comprises a loading device 2100c that holds one or more orthopedic devices 2120 in a non-straightened configuration until the orthopedic device 2120 is straightened for delivery through the lumen of a cannula or needle 2104c into the delivery site in the patient's body. The loading device 2100c may comprise a proximal end 2102c, a distal end needle 2104c and a loop 2106c with a lumen (or channel) extending there through. The loader 2100c may have a connector at its proximal end 2102c attachable to a handle, plunger, advancing structure or other loader structure. The needle 2104c may be an extension of the distal end of the loader 2100c. In one embodiment, the loader 2100c tube is rigid so an operator is able to handle it without flexing or spreading as a plunger, pusher, or advancing mechanism advances the orthopedic device 2120 through the loader 2100c.

In one embodiment of a loader, the loader may be made of a material different or dissimilar from the prosthesis or orthopedic device to avoid sticking or jamming or cross linking during a sterilization cycle. In one embodiment, a metal such as stainless steel could be used with a friction reducing layer, such as a Teflon liner or coating.

The shape of the lumen extending through the loader 2100c may be configured to orient the orthopedic device 2120 is a desired proper orientation for implantation into the body. In one embodiment, the lumen can be circular to accommodate a circular cross section orthopedic device. The overall arcuate configuration of the orthopedic device may orient the orthopedic device within the loader with a specific orientation. In other embodiments, the lumen of the loader can have a specific cross-sectional shape, key or configuration at one or more points along the loader, or along the entire length of the loader, to orient the orthopedic device in a specific orientation for delivery or retrieval.

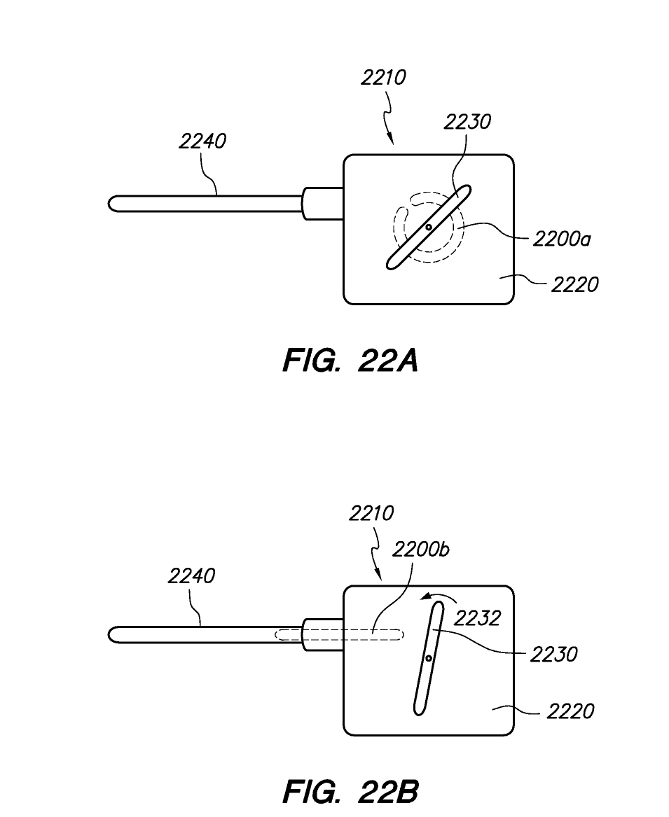

One embodiment of an orthopedic device delivery system 2210 comprises a loading device 2220 (e.g. a device loader or cassette) that holds one or more orthopedic devices 2200a in a non-straightened configuration until the orthopedic device 2200a is straightened for delivery through the lumen of a cannula 2240 or needle into the delivery site in the patient's body. In one embodiment, as illustrated in FIGS. 22A and 22B, the orthopedic device delivery system 2210 comprises a loading device 2200 (cassette) that contains and/or stores one or more orthopedic devices 2200a in a curved configuration. The loading device 2220 can be single use, disposable, or re-usable. The loading device 2220 may be removably attachable to any previously described delivery or retrieval system, including embodiments with plungers, etc. or with any embodiment described in conjunction with FIGS. 10A to 15C. As illustrated, the embodiment of the loading device 2220 may be removably attachable to a needle or cannula using any attachment configurations, such as a snap fit, lock, threaded engagement, or form fit. In one embodiment, the loading device 2220 may comprise an advancement mechanism, such as by spring loading or manual advancement, such as using a knob 2230 or other actuator mechanism to advance the device. When the knob 2230 is rotated or actuated, the orthopedic device 2200a is loaded into the lumen of the needle 2240 in a specific, proper orientation, and changes configuration to a more straightened form as shown with orthopedic device 2200b. The various embodiments of loading devices can be used with any of the embodiments of the orthopedic devices described herein.

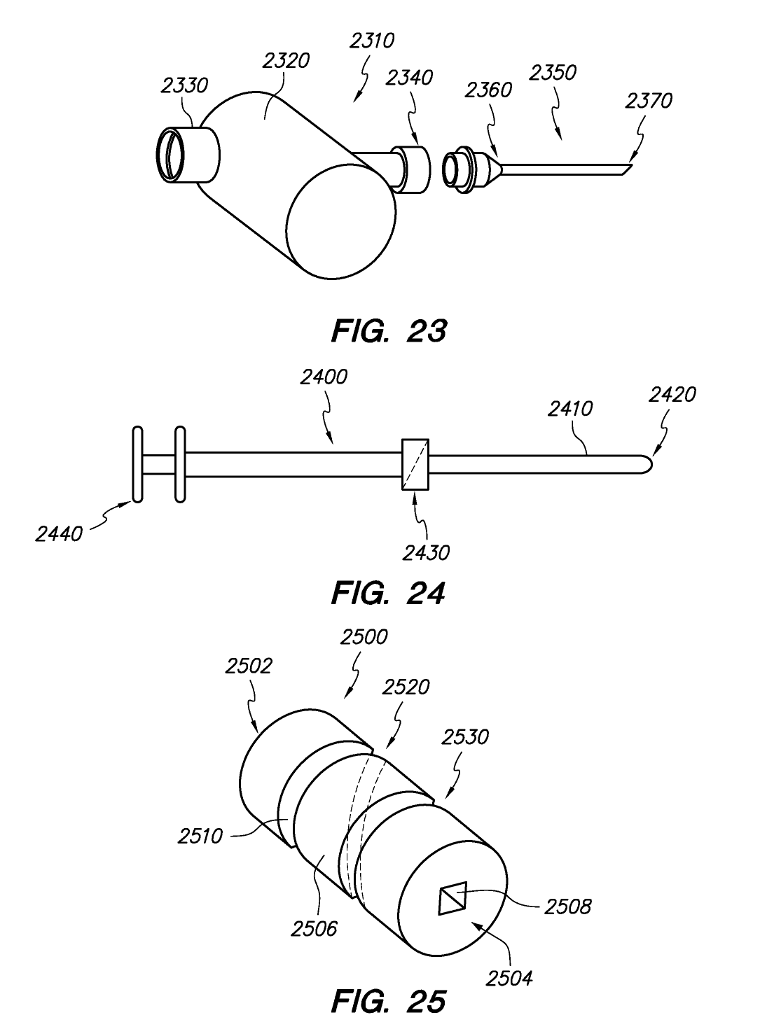

One embodiment of an orthopedic device delivery system 2310 comprises a cassette 2320 with a proximal interface 2330 and a distal interface 2340. The cassette 2320 can hold one or more orthopedic devices in a curved, rounded or rectilinear configuration until the orthopedic device is straightened for delivery through the lumen of a needle 2350 or needle into the delivery site in the patient's body. As illustrated in FIG. 23, one embodiment of an orthopedic device delivery system comprises a proximal interface 2330 that can mechanically and releasably connect to an orthopedic device advancement mechanism such as a plunger in a handle. A distal interface 2340 can mechanically and/or releasably connect to any embodiment of needle or cannula described herein. A needle 2350 may have a distal end 2370 for insertion into a joint, and a proximal interface 2360 which connects to the distal interface 2340 of the cassette 2320.

Various embodiments of an orthopedic device advancement mechanism, such as a plunger in a handle, may be employed to move the orthopedic device proximally or distally depending on the interface between the plunger and the orthopedic device. Various interfaces are discussed above. In various embodiments, the handle may be used to move the device into the delivery needle from a cassette. In certain embodiments, the plunger itself is flexible and has the same or similar diameter as the orthopedic device. The plunger may also be of a specific or fixed length such that it advances the orthopedic device to its particular position within the needle. One embodiment of a plunger 2400 is illustrated in FIG. 24, comprising a proximal end 2440 and a distal end 2410. The proximal end 2440 may be manually or mechanically advanced, and in one embodiment, may be similar to the proximal end of a plunger for use in a hypodermic syringe. In one embodiment, the proximal end 2440 and the distal end 2410 may be removably attachable at an interface 2430, while in other embodiments, the proximal end 2440 and the distal end 2410 are permanently attached. In one embodiment, the distal end 2410 is a flexible member configured to fit within a lumen of a needle to slidably advance an orthopedic device through the lumen. In various embodiments the distal end 2410 may be rigid or bendable and may have a distal tip 2420 of the distal end 2410 that is blunt and/or releasably attachable to the orthopedic device.

In one embodiment, a cassette barrel 2500 may be configured to couple with a cassette and engage the orthopedic device in such a way as to position the device in the proper orientation for implantation or extraction. FIG. 25 illustrates one embodiment of an interior component of a cassette in an orthopedic device delivery system. In one embodiment, the cassette barrel 2500 comprises a first side 2502 and a second side 2504 with a generally or substantially cylindrical surface 2506 in between. A groove 2510 on the cylindrical surface 2506 may be used to contain and guide the orthopedic device and/or a flexible plunger or a plunger tip 2410 as described above. In one embodiment, the plunger slides or travels in the helical groove 2510 shown in the “barrel.” In one embodiment, the orthopedic device is loaded into the helical pitch or groove 2510. In one embodiment, the proximal end of the orthopedic device matches the entry site of the handle connector 2330 corresponding to a portion of the groove labeled 2520. The distal end of the orthopedic device may be aligned with the delivery needle connection 2340 corresponding to a portion of the groove labeled 2530. The helical pitch of the groove 2510 may be deep enough to accept the full diameter (cross-section) of the orthopedic device. For instance, if the device is a 2 mm device the groove 2510 width and/or depth can be a little larger than 2 mm. In one embodiment, the groove 2510 also accepts the full diameter (cross-section) of at least a portion of the plunger 2400, such as the distal portion 2410, which travels within the groove 2510 to push the proximal end of the orthopedic device distally.

In one embodiment, the overall barrel 2500 diameter within the groove 2510 can be larger, smaller or equal to the normal shape or diameter of a rounded (or non-straightened) configuration of the orthopedic device. The groove 2510 can be used to hold the orthopedic device in the cassette 2320 at or near its round (normal) shape. Once the orthopedic device is pushed or pulled from the cassette 2320 into a cannula or needle such as needle 2350, the orthopedic device can assume a straight or slightly curved shape. In one embodiment the helical groove 2510 is configured to hold the orthopedic device in its normal non-straightened shape, with the ends of the orthopedic device offset so it can be pushed at its proximal end to advance its distal end.

In one embodiment, the groove 2510 may be configured to aid in the saline flush for lubrication of the orthopedic device. The configuration may include micro grooves along the walls of the groove 2510 itself. In one embodiment, the system could be flushed with sterile saline prior to orthopedic device delivery through the handle connector. Additionally, the groove 2510 in the barrel 2500 may have one or a plurality of micro grooves along its length allowing for a substance, such as silicone, to be flushed when the saline is injected.

In one embodiment, the barrel 2500 is contained by the outer housing 2320. In one embodiment the barrel 2500 is keyed to assure proper orientation within the outer housing 2320. The first side 2502 and/or the second side 2504 may have a key slot 2508 at or near an axis. In one embodiment, the cassette barrel 2500 is contained within the cassette housing with an external handle or knob connected to the key slot 2508 in order to rotate the cassette barrel 2500, thereby advancing or retracting an orthopedic device. In another embodiment, the cassette barrel 2500 may held by the cassette housing with an interlocking key feature on the inside of the cassette housing which locks the key slot 2508 so that the cassette barrel 2500 does not rotate. Note that the illustrated square “key” configuration shown at the center of the barrel (or other mechanical interfit configuration) may be used if the barrel is a separate component to the cassette housing assembly and would has a particular orientation when assembled to the cassette. In another embodiment, the cassette 2320 can be comprised of an integrated housing and barrel structure, where the handle/plunger and cassette can be one-piece (e.g. permanently attached or formed into a single structure). Various needles can be attached depending on the desired size, indication, and orientation intended for the implant.

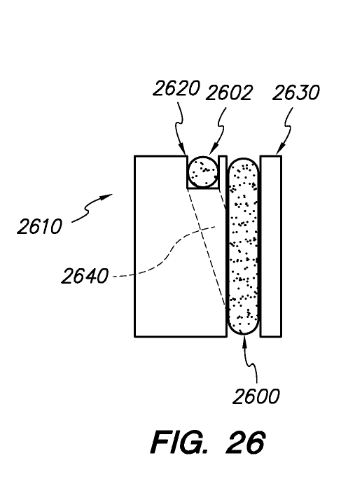

Another view of a barrel 2610, which can be similar to the barrel 2500, is shown in FIG. 26. In one embodiment, a groove 2620 can be configured to house or guide an orthopedic device 2600 with a proximal portion 2602. A portion of the groove can be slanted 2640, curved, straight 2630, spiral, helical or some other shape to ensure the orientation of the orthopedic device 2600 is proper for delivery to the patient. In one embodiment a plunger, as described above, can be housed in the groove 2610 for slidably advancing or retracting the orthopedic device 2600. In various embodiments, the groove 2610 may be oversized to house the orthopedic device or plunger, or may have a square or rounded cross sectional shape or any other configuration. The groove 2610 may also be configured to direct the orthopedic device 2600 along a path which terminates at a feature which guides (straightens) the orthopedic device 2600 for insertion into the delivery needle.

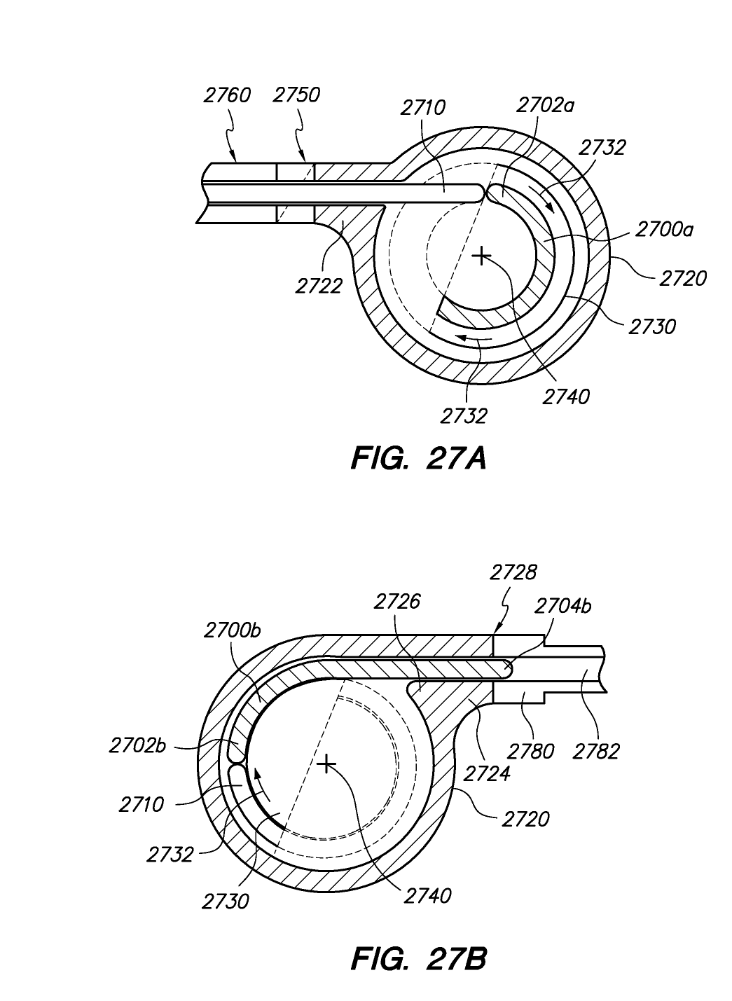

As described above, in one embodiment, a flexible plunger can be used to advance or retract an orthopedic device through a cassette and into a straight or straightened configuration for delivery through a needle. FIGS. 27A and 27B are partial cut-away schematic side views of one embodiment of the distal advancement of an orthopedic device 2700a and 2700b from a cassette 2720 and into a needle 2780 that is fixedly or removably attachable at a connection 2728 near the distal end 2724 of the cassette 2720. A plunger 2710 may be advanced through a lumen in a handle or advance mechanism 2670. The advance mechanism 2760 and the cassette 2720 may be fixedly or removably attachable at a connection 2750 near the proximal end 2722 of the cassette 2720. The plunger 2710 may be advanced into a groove along a barrel 2730 with a center 2740 to advance the proximal end 2702a of the orthopedic device 2700a (which is also shown as proximal end 2702b of the orthopedic device 2700b in FIG. 27B) in a direction 2732. The distal end 2704b of the orthopedic device 2700b may be advanced over an alignment ledge 2726 near the distal end 2724 of the cassette 2720. As the distal end 2704b of the orthopedic device 2700b advances in the lumen 2782 of the needle 2780, the orthopedic device 2700b may straighten out into a slight curve or a straight line configuration, for example. In various embodiments there could be more than one orthopedic device per cassette or multiple cassettes joined together.

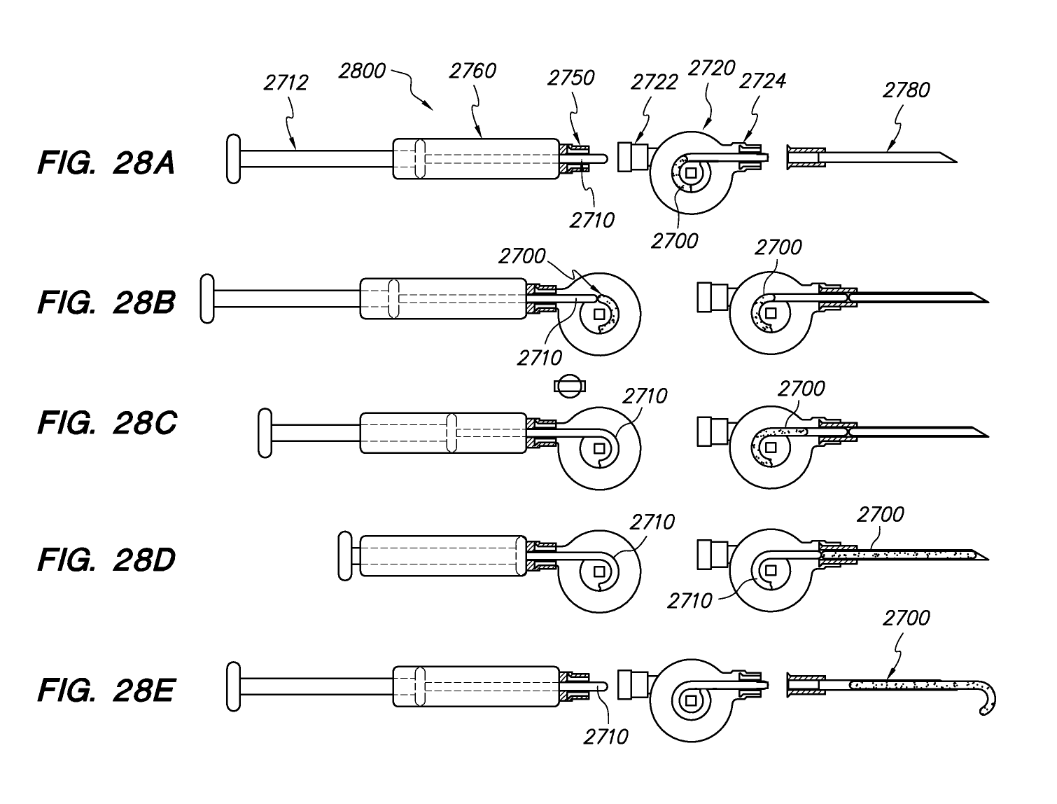

One embodiment of an orthopedic device delivery system 2800 comprises a cassette, a barrel and a plunger that is shown in FIGS. 28A to 28E, illustrating the advancement of an orthopedic device 2700 using a plunger advancer 2712 in a plunger body 2760 having a plunger luer connector 2750 and a plunger distal portion 2710. The cassette body 2720 has a proximal plunger luer connector 2722 and a distal delivery needle luer connector 2724 that is removably attachable to a delivery needle 2780. As the plunger advancer 2712 moves distally, the flexible plunger distal portion 2710 advances into the cassette 2720 and bends around to push the orthopedic device 2700 out of the cassette 2720 and into the needle 2780. The distal tip of the plunger distal portion 2710 advances forward pushing the proximal end of the orthopedic device 2700 along a groove provided in a cassette barrel as shown in FIGS. 23 to 27. The plunger 2712 advances the device 2700 completely into the needle 2780 to a predetermined depth. In one embodiment the device 2700 is detached from the plunger distal portion 2710 (with any of the embodiments of attachment mechanisms described herein, such as with FIG. 19 or 20) and the needle 2780 is withdrawn from the implant delivery site.

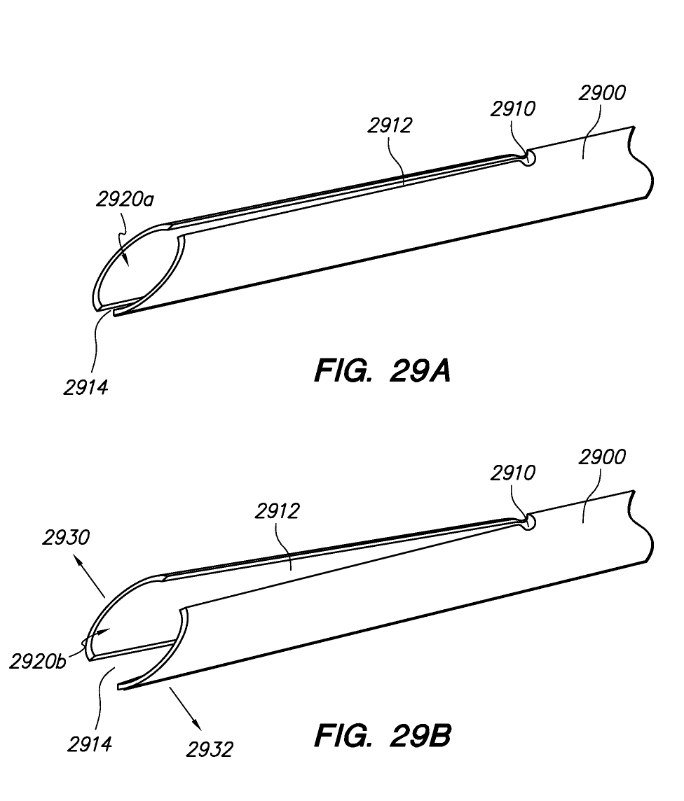

In one embodiment, a slotted needle 2900 that can be used with any of the orthopedic delivery device systems described may be used to spread the bones of a joint apart as the delivery device is advanced into the joint. In the embodiment illustrated in FIGS. 29A and 29B, the slotted needle 2900 has a stress relief 2910 and at least a first slot 2912. In the depicted embodiment, the slotted needle 2900 also has a second slot 2914. In one embodiment, the lumen or bore of the needle may or may not be undersized relative to the prosthesis or orthopedic device. The needle may be advanced into the joint at a small diameter. Then, when the prosthesis is advanced through the bore, it forces the split barrel of the needle outwards. This outward force may be sufficient to influence/spread bone or tissue outwardly from the centerline of the needle. In the illustrated embodiment, the needle lumen expands from a first configuration 2920a to a second configuration 2920b, where the sides of the needle at the distal end of the slotted needle 2900 can be moved apart in directions indicated by arrows 2930 and 2932. The distal slotted end of the needle can also be narrowed so that it slips in between the bones. Then, as the orthopedic device is advanced distally, it urges (or pushes) the bones of the joint apart. In one embodiment, a radiopaque strip or marker can be provided on one or both edges of the slot for orientation. In various embodiments, one or more slots 2912 and/or 2914 may have a length of about 0 to about 100 mm, may have a slot width from 0.001 thousandths of an inch to 0.025 thousandths of an inch, may have a slot width that can vary from slot to slot or along the individual slot, may be straight, can be curved, may be spiral, and/or may have a proximal end of the slot that is provided with a pivot to allow for springing apart without cracking.

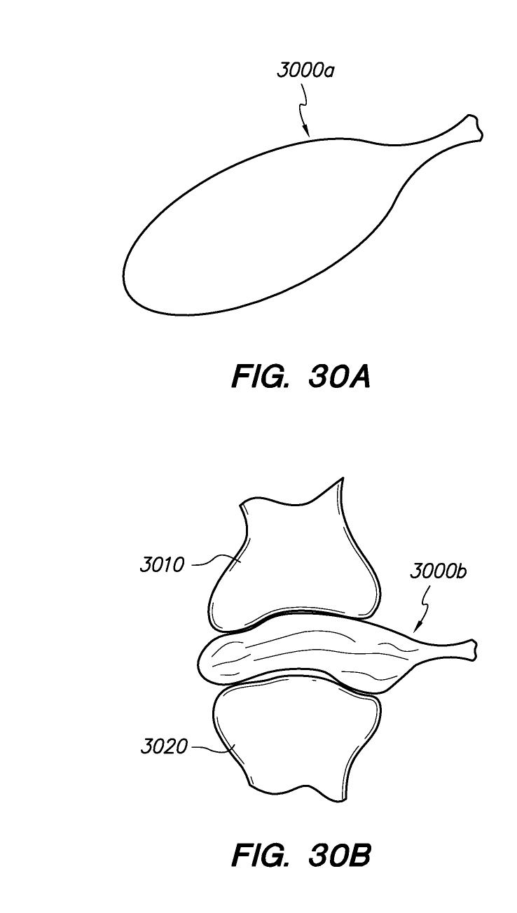

In one embodiment, an orthopedic delivery device system may comprise a balloon 3000a that may be used to spread the bones of a joint apart as the delivery device is advanced into the joint. In the embodiment illustrated in FIGS. 30A and 30B, a balloon may be configured for use with specific joints, such as finger joints, can be combined with any of the devices or systems described herein. The balloon 3000 (shown in one embodiment at least partially inflated in 3000a and deflated in 3000b) can be a high pressure balloon and may be delivered through a cannula or needle, or be attached to the end of a needle. The balloon 3000b may be inserted between the bones and inflated using techniques similar to traditional angioplasty techniques, for example. In one embodiment, a spoon or football shape would allow for easy insertion through a large bore needle as intended for the procedure. Once inflated, the balloon 3000a may assume the unique shape of the bones and may also be used stretch the joint capsule. In one embodiment, the balloon 3000 may have a small profile when inserted, for example about 5 French or less (e.g. about 0.067 inches or about 1.67 mm or less in diameter). The balloon 3000 may be inflated or expanded up to about 15 French (e.g. up to about 0.197 inches or about 5 mm in diameter). In one embodiment, the balloon 3000 may be pleated or folded. In one embodiment, the balloon 3000 may be removed or may remain in place during device delivery, and can undergo partial or complete inflation or deflation during the device delivery.

In one embodiment of an orthopedic device delivery system, a sizing template for joint evaluation and device size may be provided to facilitate the determination of the implant size. In other embodiments, a separate sizing instrument may be used before selection of an orthopedic device and/or an orthopedic device delivery system.

One embodiment of an orthopedic device delivery system comprises a delivery handle and a loading device that can at least partially straighten an implantable device or implant, then eject the implantable device or implant in a controlled and defined plane into a joint of the body of a patient. The delivery handle may be configured to advance a plunger (or push rod or advance rod) as previously described herein, and may be shaped so that the orientation of any of the embodiments of delivery channels, needles, cannulas, and similar structures are directed for a particular alignment and orientation for implantation or extraction. One embodiment of a loading device may be configured to advance an orthopedic implant into the delivery channel, needle, cannula, or similar structures. For example, a cannula may be configured to be fixed to a handle so that the cannula cannot move with respect to the handle, thereby providing a particular orientation of the implant with the delivery device. In one embodiment, the cannula may be locked in place and fixed with respect to a handle, and in another embodiment, the cannula can be permanently fixed to the handle.

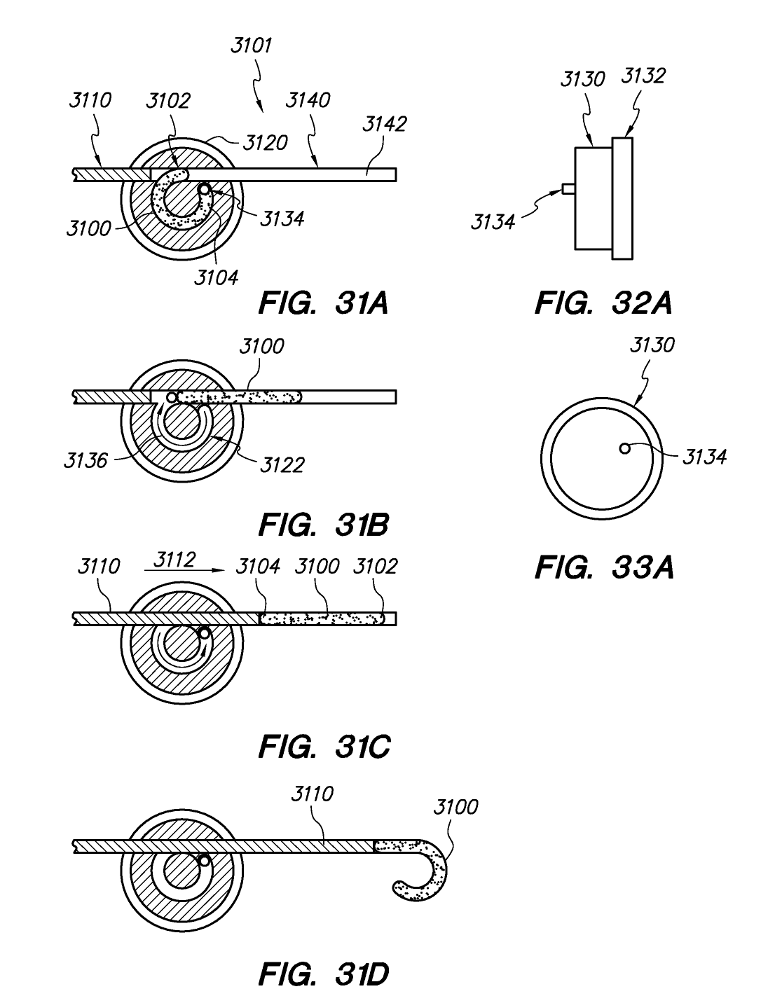

Various embodiments of an orthopedic device delivery system may comprise a handle, delivery mechanism (e.g. a plunger, push rod, or advance rod) and a loading device (e.g. a device loader or cassette) that holds one or more orthopedic devices in a non-straightened configuration until the orthopedic device is straightened for delivery through the lumen of a cannula into the delivery site in the patient's body. Certain embodiments of this type of orthopedic device delivery system are illustrated in FIGS. 31A to 41C. The handle, which can be held by a medical practitioner, may provide linear translation (forwards or backwards) of the plunger. In various non-limiting embodiments, the translation of the plunger may be accomplished by a number of different mechanisms or structures, such as a rotating knob, a trigger, an indexing mechanism (similar to a mechanical pencil), a hand gun, a ratcheting type mechanism, a screw type mechanism, and/or a rack and pinion. The delivery mechanism, or plunger, can be flexible, semi-rigid or rigid, as previously disclosed above, and may be configured to move an orthopedic device through a lumen in a cannula for delivery or retrieval to or from a patient's body.

In one embodiment, as illustrated in FIGS. 31A to 31D, an orthopedic device delivery system 3101 comprises a plunger 3110, a loading device 3120 (that can also be called a cassette) and a cannula 3140. The loading device 3120 comprises a storage delivery channel 3122 that contains and/or stores one or more orthopedic devices 3100 in a native, non-straightened configuration. In one embodiment a natural configuration of an orthopedic device is arcuate, or a curved configuration. The orientation of the orthopedic device 3100 for delivery into a patient may be set by configuring the system 3101 such that the orthopedic device 3100 exits the cannula 3140 in an orientation or plane substantially parallel to an articular bone surface in a joint. The orthopedic device 3100 may comprise a distal end 3102 and a proximal end 3104. The orthopedic device delivery system 3101 may at least partially straighten the orthopedic device 3100 for accurate delivery by assuring stability and proper orientation upon deployment. The loading device 3120 may be single use, disposable, or re-usable. The loading device 3120 may be removably attachable to any previously described delivery or retrieval system, including embodiments with plungers, etc. or with any embodiment described in conjunction with FIGS. 10A to 15C and 21A to 30B. As illustrated in FIGS. 31A to 31D, the embodiment of the loading device 3120 may be fixed to a cannula 3140 using any attachment configurations, such as permanent fixation, bonding, a snap fit, lock, threaded engagement, form fit, bonding, or mechanical locking mechanism to fix the orientation of the cannula 3140 with respect to the loading device 3120 for proper alignment and orientation for delivery or retrieval of an orthopedic device 3100. In one embodiment, the loading device 3120 comprises an advancement mechanism, such as by spring loading or manual advancement, such as using a knob 3130 (one embodiment is illustrated in alternate views in FIGS. 32A and 33A) to advance the orthopedic device 3100. One embodiment of a knob 3130 comprises an actuation surface 3132 for rotation or manipulation to move the knob 3130 and a delivery pin 3134. The delivery pin 3134 may be fixed to the knob 3130. Rotation or actuation of the knob 3130 moves the delivery pin 3134 within the delivery channel 3122, resulting in the movement of the proximal end 3104 of the orthopedic device 3100 into a lumen 3142 of the cannula 3140. In the illustrated embodiment, rotation of the knob 3130 moves the delivery pin 3134 in a clockwise motion indicated by arrow 3136, but in other configurations, the delivery pin may move in a counterclockwise motion.

FIG. 31A illustrates the device in its loaded, unstrained or reduced strain state. In various embodiments, the distal end 3102 of the orthopedic device 3100 may rest within the delivery channel 3122 or protrude in to the lumen 3142 of the cannula 3142. Operation of the loading device 3120 brings the orthopedic device 3100 from the delivery channel 3122 into a position to be actuated by the plunger 3110 through the lumen 3142 of the cannula 3140 for delivery into the patient. The delivery channel 3122 may be in communication with the lumen 3142 of the cannula 3140. The distal 3102 or leading end of the orthopedic device 3100 may be positioned in or near the entrance to the lumen 3142 of the cannula 3140 and the proximal end 3104 may rest against the delivery pin 3134.

When the knob 3130 is rotated or actuated as illustrated in FIGS. 31B to 31C, the orthopedic device 3100 is loaded into the lumen 3142 of the cannula 3140 in a specific orientation, and changes in configuration to a more straightened form inside the lumen of the cannula 3140. The various embodiments of loading devices can be used with any of the embodiments of the orthopedic devices described herein. As the knob 3130 is rotated clockwise, it forces the delivery pin 3134 against the proximal end 3104 and pushes the orthopedic device 3100 clockwise through the delivery channel 3122 to the lumen 3142 of the cannula 3140 where the orthopedic device 3100 is straightened. The orthopedic device 3100 is then moved from its normal arcuate configuration in to a substantially straightened configuration while maintaining its proper orientation for delivery. The knob 3130 can be spring loaded to return to its initial position when the actuation surface 3132 is released. FIGS. 31C and 31D illustrate the plunger 3110 being advanced distally along the arrow marked 3112 within the lumen 3142 of the cannula 3140 to advance the orthopedic device 3100 out of the cannula 3140 and into the patient in an orientation for proper delivery. One embodiment of the orthopedic device delivery system 3101 provides for the orthopedic device 3100 to exit the cannula 3140 in a plane that is substantially the same as the original loaded state within the delivery channel 3122 to help provide a particular deployment orientation. As the orthopedic device 3100 exits the lumen 3142 of the cannula 3140, the orthopedic device 3100 can return to its arcuate configuration, in a clockwise direction and in a plane that is substantially the same or parallel to the plane of the delivery channel 3122 within the a loading device 3120. With the proper orientation of the orthopedic device delivery system 3101, the orthopedic device 3100 can be delivered in a plane substantially parallel to a plane between the articulating bony or cartilaginous surfaces within a joint.

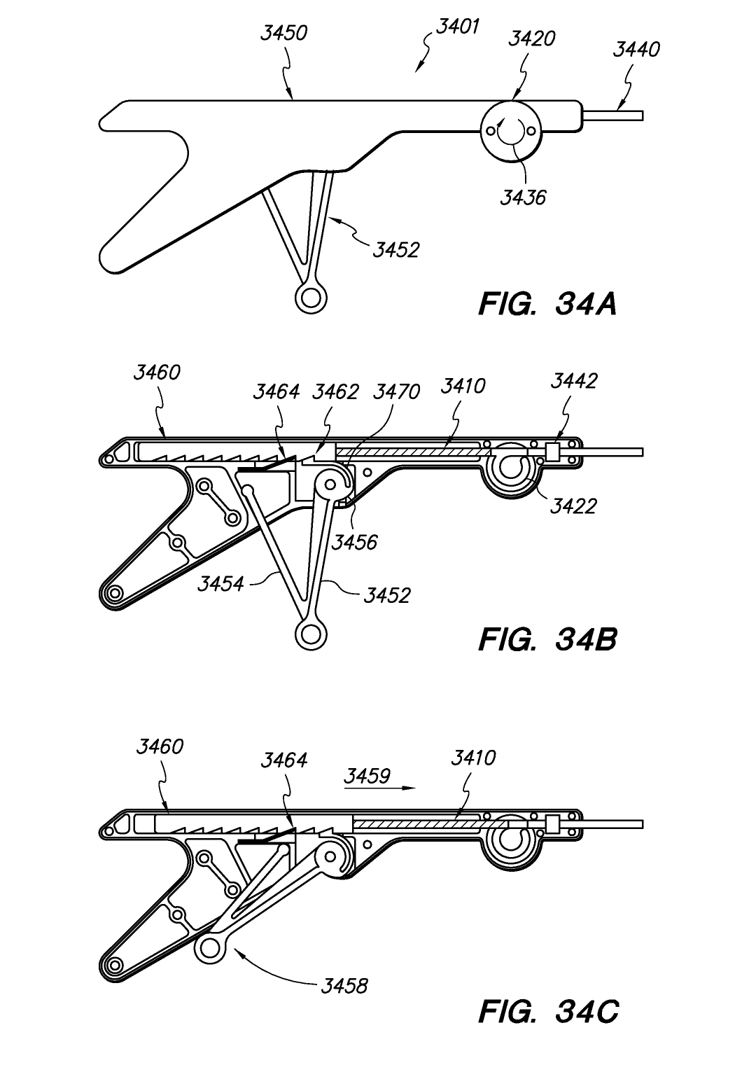

In one embodiment, as illustrated in FIGS. 34A to 34C, an orthopedic device delivery system 3401 comprises a cannula 3440, a loading device 3420 with delivery channel 3422, and a handle 3450 with a pistol grip configuration. The handle 3450 comprises a trigger 3452 configured to advance the plunger 3410 with a rack and pinion linear advancement mechanism. In one embodiment, the one-piece, trigger 3452 comprises a trigger head 3456 and a trigger return 3454. Movement of the trigger 3452 in the direction indicated by arrow 3458 engages the rack 3460. The rack 3460 comprises at least a first ratchet head 3462 and additional ratchet heads, or teeth. A ratchet 3464 is configured to engage the ratchet heads 3462 along the rack 3460 to prevent the rack 3460 from moving backwards during advancement of the plunger 3410. The trigger 3452 engages the rack 3460 at the first ratchet head 3462. When the trigger 3452 is pulled back, the ratchet arm 3470 winds down around the trigger head 3456 and pulls the ratchet tooth 3462 along with the rack 3460 distally in the direction of arrow 3459 to linearly advance the plunger 3410. The construction of the ratchet arm 3470 to the trigger head 3456 is such that the cantilever provides a spring force and resists downward deflection. Upon release of the trigger 3452, the ratchet arm 3470 releases its potential energy and returns the trigger 3452 to a neutral position (forward as illustrated in FIGS. 34A and 34B). As the trigger 3452 returns, the ratchet head 3462 advances distally along arrow 3459 and the ratchet arm 3470 biases down until the ratchet arm 3470 can spring back into the next tooth, or ratchet head 3462. In one embodiment, the cannula 3440 may be locked to the handle 3450 with a cannula lock 3442. In one embodiment, the cannula 3440 and cannula lock 3442 may be integrally formed.

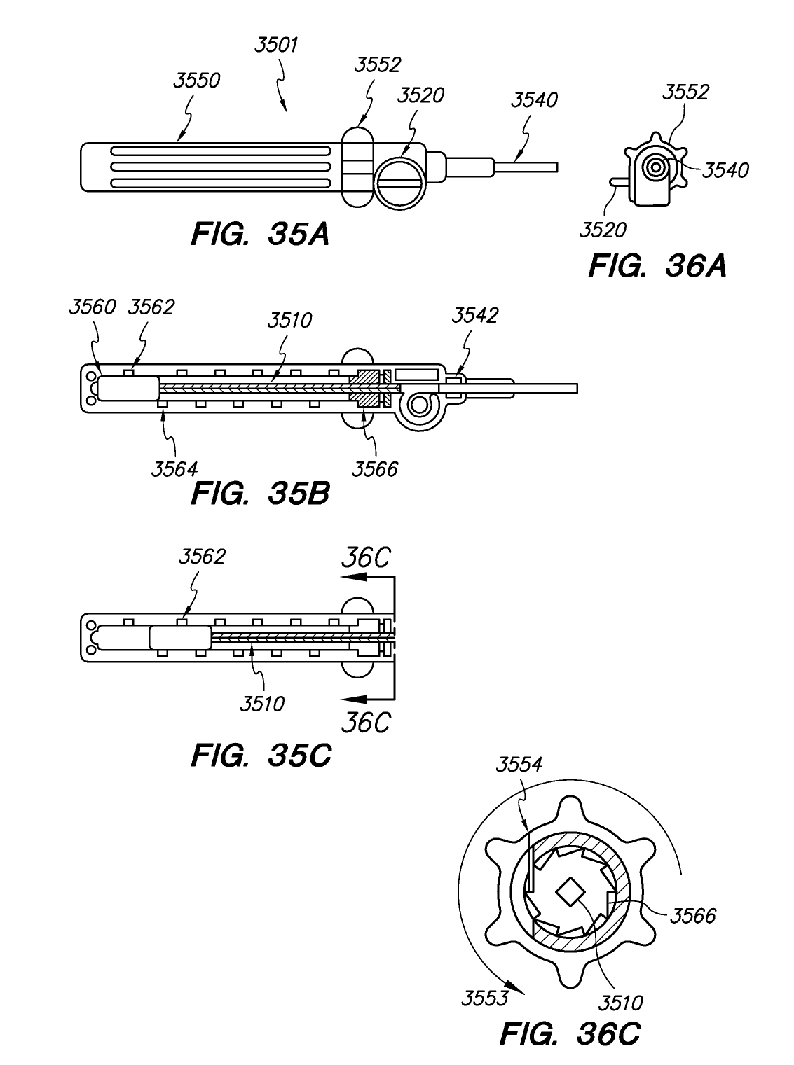

In one embodiment, as illustrated in FIGS. 35A to 35C and FIG. 36A, an orthopedic device delivery system 3501 comprises a cannula 3540, a loading device 3520, a delivery knob 3552 and a handle 3550. The cannula 3540 may be similar to previously described cannulas, and in the illustrated embodiment, includes a cannula lock 3542. The loading device 3420 may be similar to embodiments of the loading device knob described above. The delivery knob 3552 interacts with a follower 3560 and a follower pin 3562. The follower 3560 may be attached to a non-circular cross-section push rod 3510. The push rod 3510 may have a square cross-section, but it may also have any cross-sectional shape that allows it to rotationally engage the inside of the delivery knob 3552 while still free to slidably actuate or move axially through the axis of the delivery knob 3552. Rotation of the delivery knob 3552 rotates the push rod 3510, as viewed in FIG. 36A or 36C, and causes the follower pin 3562 to move through a helical track 3564 disposed inside the handle 3550 around the follower 3560. This results in axial movement of the follower 3560 and push rod 3510 to advance the orthopedic device out of the lumen of the cannula 3540.

FIG. 36C illustrates an embodiment of drive system for a follower 3560. The push rod 3510 may comprise a cross-section shape that is square or some shape other than round, and slides axially through the ratchet drive 3566. The ratchet drive 3566 can spin freely inside the handle 3550. The delivery knob 3552 comprises a ratchet pawl 3554 that engages the ratchet drive 3566 and is secured to the handle 3550 for rotational actuation. As the delivery knob 3552 is turned in the direction of the arrow marked 3553, the rotation indexes the ratchet drive 3566 counter-clockwise as viewed from the end of the orthopedic device 3501 and as illustrated in FIG. 36C. Because of non-circular shape of the push rod 3510 and the complementary shape of the hole through the axis of the ratchet drive 3566, the rotation of the delivery knob 3552 also turns with the ratchet drive 3566. This causes the push rod 3510 to rotate as well. When the push rod 3510 rotates, it twists the follower 3560 through the track 3564 via the follower pin 3562. Since the push rod 3510 is not axially fixed, the push rod 3510 moves distally as the follower 3560 is spun. The delivery knob 3552 may be manually returned by twisting it in the opposite direction. This causes the ratchet pawl 3554 to disengage and subsequently fall into the next tooth in the ratchet drive 3566. The process can repeat itself until the follower 3560 abuts against the proximal end of the ratchet drive 3566.

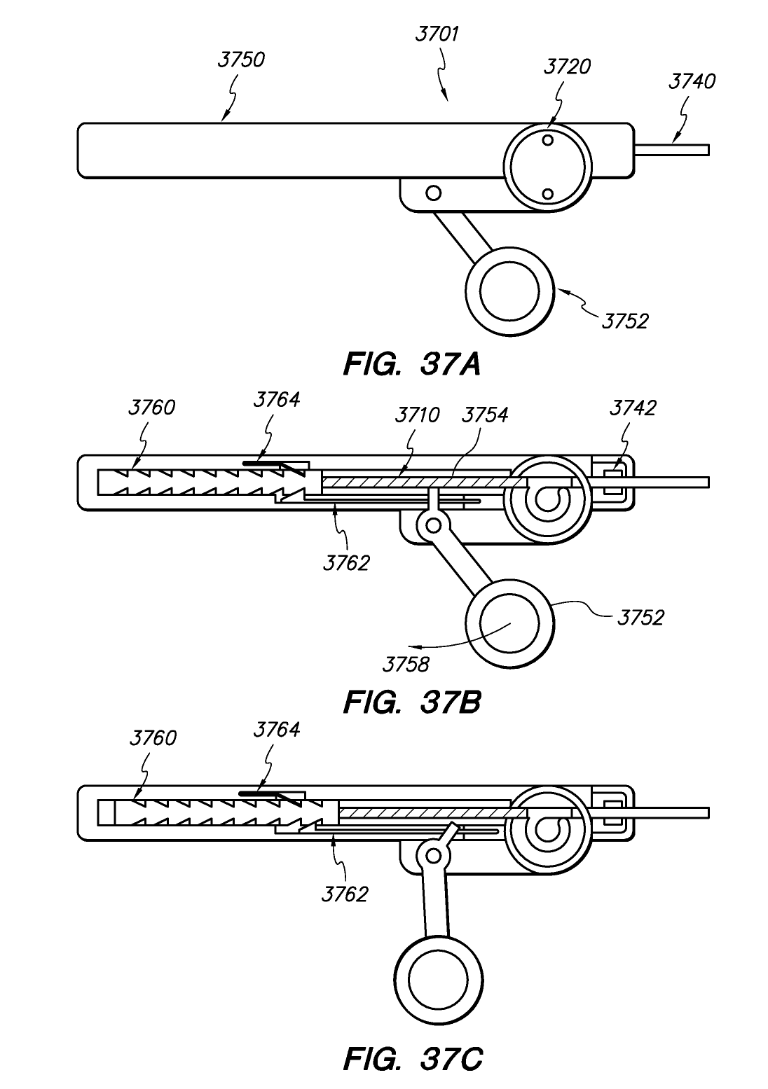

In one embodiment, illustrated in FIGS. 37A to 37C, an orthopedic device delivery system 3701 comprises a cannula 3740, a loading device 3720, a handle 3750 and a finger-loop trigger 3752. The cannula 3740 may be similar to previously described cannulas, and in the illustrated embodiment includes a cannula lock 3742. The handle 3750 may comprise a trigger 3752 configured to advance the push rod 3410 with a rack and pinion linear advancement mechanism. The rack 3760 has teeth on both sides as illustrated in FIGS. 37B and 37C. The secondary ratchet 3764 engages the top set of teeth and keeps the rack 3760 from sliding back when the primary ratchet 3762 is returned to its starting position. The trigger 3752 may have a trigger pawl 3754 that engages the primary ratchet 3762. The primary ratchet 3762 has at least one through hole or slot for the trigger pawl 3754 to fit within. As the trigger 3752 is pulled back in the direction indicated by arrow 3758, the trigger 3752 causes the trigger pawl 3754 to pivot forward in the distal direction pushing the slot in the primary ratchet 3762 forward in the distal direction. This in turn causes the primary ratchet 3762 to move forward and move the rack 3760 forwards. The secondary ratchet 3764 then engages the next tooth. When the trigger 3752 is moved to the forward or starting position, it moves the primary ratchet 3762 backwards via the trigger pawl 3754 to engage the next tooth.

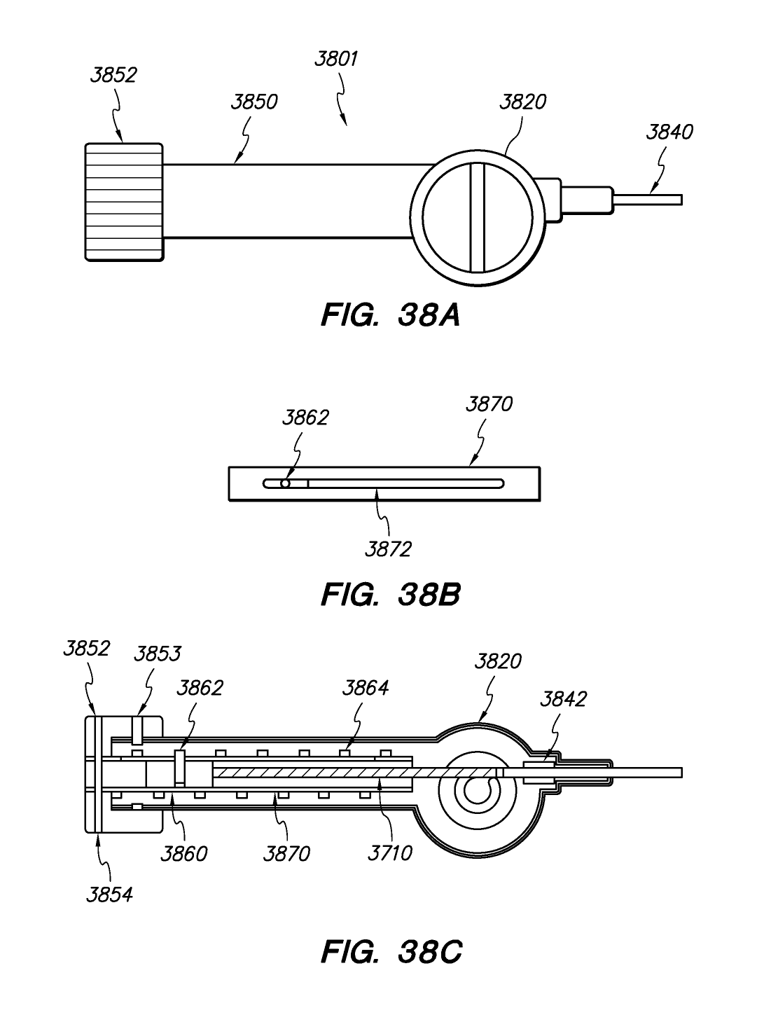

In one embodiment, illustrated in FIGS. 38A to 38C, an orthopedic device delivery system 3801 comprises a cannula 3840, a loading device 3820, a proximal delivery knob 3852 and a handle 3850. The cannula 3840 may be similar to previously described cannulas, and in the illustrated embodiment includes a cannula lock 3842. The loading device 3820 may be similar to embodiments of the loading device knob described above. The proximal delivery knob 3852 may be attached to a rotating advance tube 3870 that drives a follower 3860 through a track 3864. The advance tube 3870 has a slot 3872 along at least a portion of the length of the advance tube 3870. The slot 3872 has a width that is slightly oversized to the diameter of the follower pin 3862. Rotation of the delivery knob 3852 rotates the advance tube 3870, which pushes the follower pin 3862 through the helical track 3864 that runs along at least a length of an interior channel in the handle 3850. The follower pin 3862 is attached to the follower 3860, which is connected to the push rod 3810. Rotation of the delivery knob 3852 causes the follower 3860 to advance along the track 3864 which results in axial movement of the follower 3860 and push rod 3810 to advance the orthopedic device out of the lumen of the cannula 3840. The knob 3852 is attached to the advance tube 3870 with an advance lock 3854. In one embodiment, the advance lock 3854 is a dowel pin. The dowel pin may ride in a groove in the body of the handle 3850. Rotating the knob 3852 in the opposite direction causes the follower pin 3862 to move proximally, or backwards, along the track 3864 causing the follower 3860 and the push rod 3810 to move proximally, or backwards, as well.

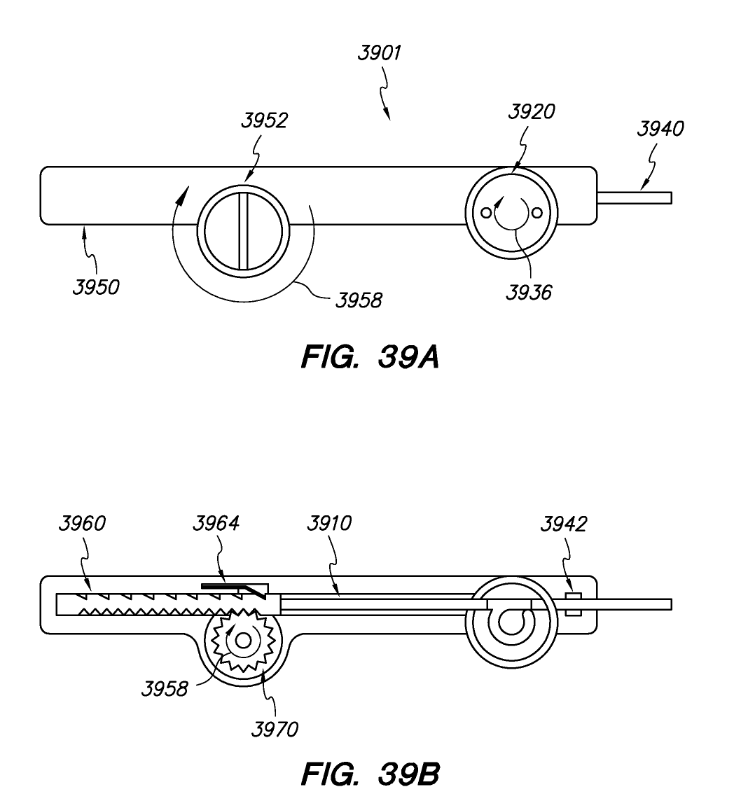

In one embodiment, illustrated in FIGS. 39A to 39B, an orthopedic device delivery system 3901 comprises a cannula 3940, a loading device 3920, a delivery knob 3952 and a handle 3950. The cannula 3940 may be similar to previously described cannulas, and in the embodiment illustrated includes a cannula lock 3942. The loading device 3920 may be similar to embodiments of the loading device knob described above, and loads an orthopedic device into the cannula 3940 when the loading device 3920 is rotated in a direction indicated by the arrow referenced in FIG. 39A as 3936. In various embodiments, the delivery knob 3952 may be positioned proximally and to a side of the orthopedic device delivery system 3901, and may be on the same side as the loading device 3920. When rotated in a direction indicated by the arrow 3958, the delivery knob 3952 provides axial movement to a push rod 3910 which pushes the orthopedic device through the cannula 3940 and into a patient. The delivery knob 3952 engages the rack 3960 by way of a drive wheel 3970, which has teeth that engage corresponding teeth on the rack 3960. A ratchet 3964 keeps the rack 3960 from moving backwards (or proximally) when the drive wheel 3970 is not turned. In one non-illustrated embodiment, the ratchet 3964 could be a part of the housing for the drive wheel 3970.

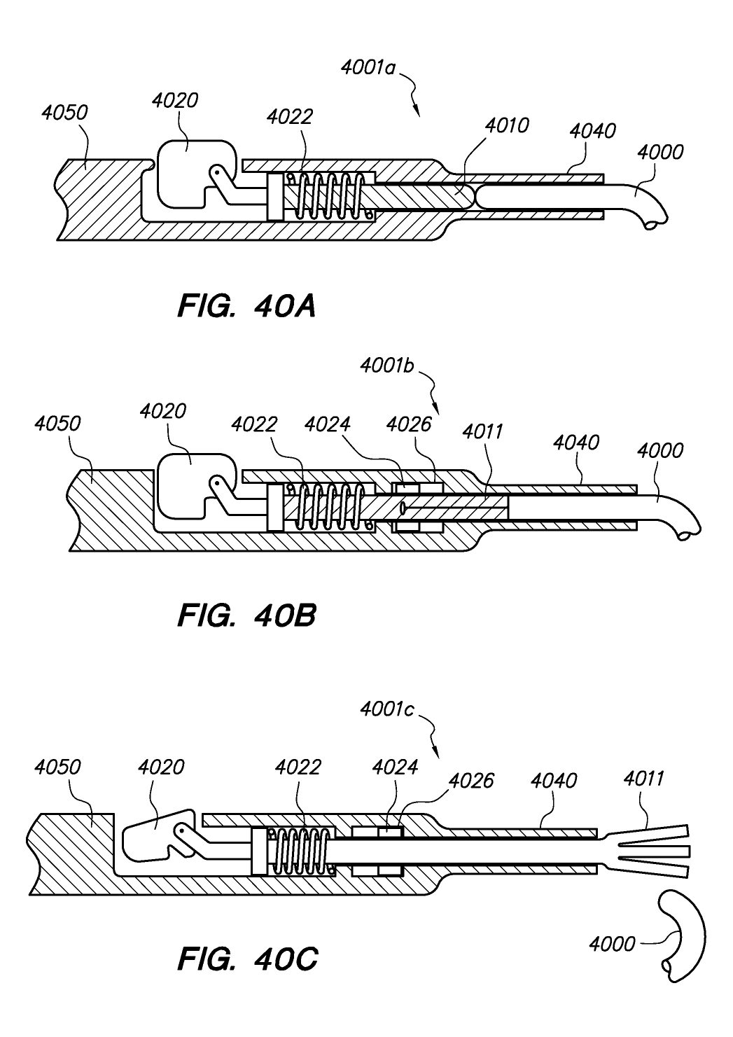

In one embodiment, as illustrated in FIG. 40A, an orthopedic device delivery system 4001 comprises a cannula 4040, a handle 4050 and a push-button 4020 to axially advance an orthopedic device 4000 distally though the lumen of the cannula 4040 into a patient. The orthopedic device delivery system 4001 may be configured for one-handed actuation, using a trigger-type mechanism. Other one-handed actuation mechanisms include rotation of a knob with a finger or thumb, or actuation of a trigger with a single hand. In one embodiment, an orthopedic device delivery system 4001a uses a mechanically actuated push rod 4010 that is in contact with the proximal end of the orthopedic device 4000. The push rod 4010 may be used to advance the orthopedic device 4000 through the cannula 4040. As depicted in FIGS. 40B and 40C, one embodiment of the delivery system 4000b and 4000c comprises a push rod 4011 connectable to an orthopedic device 4000 that can be moved proximally and/or distally though the lumen of the cannula 4040 and into a patient. Although the push rod 4011 with a connection is illustrated with a push-button orthopedic device delivery system, as shown with orthopedic device delivery system 4001a, it can be used with any other actuator mechanism or other orthopedic device delivery system described herein. Although not illustrated in FIGS. 40A to 40C, the axial advancement mechanism can be used in conjunction with any of the embodiments of loading devices, including loading knobs and cassettes described above. In one embodiment an orthopedic device delivery system 4001b comprises a push rod 4011 that includes a detachable connection between the push rod 4011 and the orthopedic device 4000, such as a releasable collet mechanism for manipulation of the orthopedic device 4000 within the patient prior to release of the orthopedic device 4000. A spring 4022 may be used to return the push rod 4010 or 4011 to a proximal position. A sleeve 4024 in an opening 4026 can allow opening and closing of the collet attachment in 4011. Pressing the push button 4020 advances the orthopedic device 4000 distally along the cannula 4040. In one embodiment, the collet attachment permits manipulation, positioning, repositioning, release, or re-capture and/or removal of the orthopedic device within the joint, if necessary. In one embodiment, the push rod 4011 is releasably attachable to the proximal end of the orthopedic device and can release the orthopedic device or recapture it. In one embodiment, the sleeve 4024 can be actuated by the user with a switch or other mechanism to release the orthopedic device 4000 from the push rod 4011 by allowing the distal end of the push rod 4011 to expand when the sleeve 4024 is in a proximal position, as shown in FIG. 40B and FIG. 40C. When the sleeve 4024 is in a distal position (not illustrated), the orthopedic device is held in the push rod 4011. In other embodiments, the sleeve can be closer to the distal end of the push rod 4011.

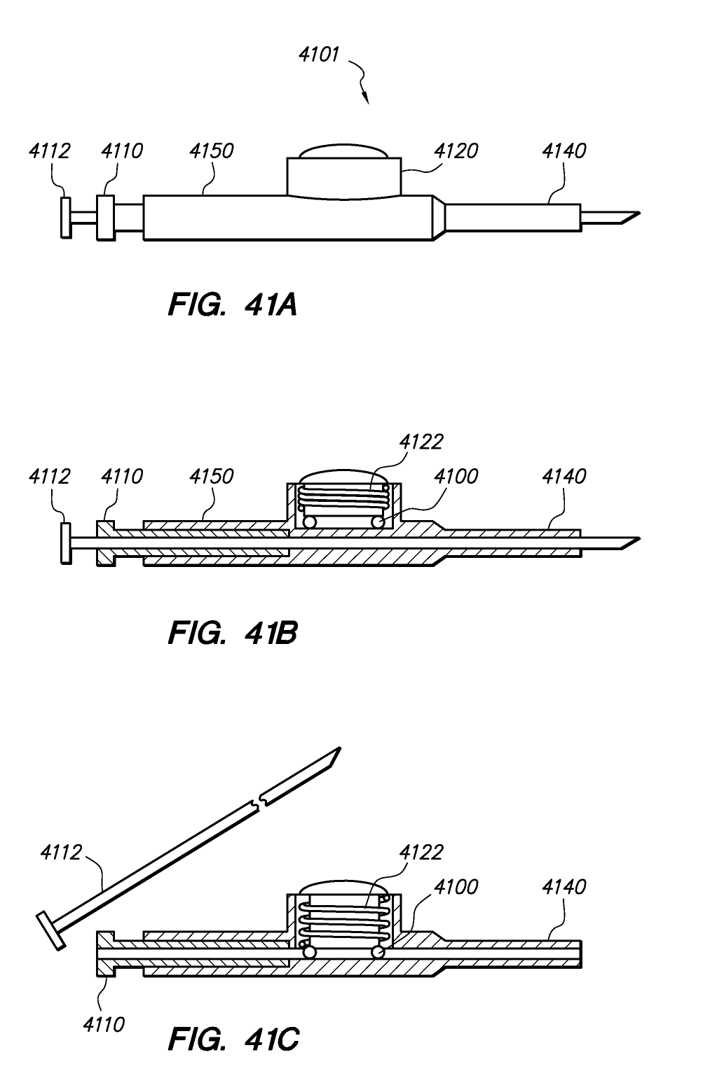

In one embodiment, as illustrated in FIGS. 41A to 41C, an orthopedic device delivery system 4101 comprises a cannula 4140, a handle 4150, a loading device 4120 and a removable tissue piercing device 4112. In one embodiment the loading device 4120 stores one or more orthopedic devices 4000 that can be actuated in line with the lumen of the cannula 4140 with a mechanism 4122, such as a spring, that will align the orthopedic device 4000 for delivery once a removable tissue piercing device 4112, such as a trocar (including solid or tubular trocars), is removed from the device after piercing tissue in a patient to access a delivery site. When the tissue piercing device 4112 is removed proximally out of the orthopedic device delivery system 4101 as shown in FIG. 41C, the loading device 4120 moves the orthopedic devices 4000 for loading into the cannula 4140.

In some of the examples described herein, the orthopedic device may be delivered to an implantation site using a delivery system that axially moves or slides the orthopedic device along it longitudinal axis, e.g. the movement pathway of the orthopedic device is generally co-axial with the longitudinal axis of the orthopedic device. In other examples, however, the orthopedic device may be delivered using a movement axis that is transverse or otherwise non-axially oriented with the longitudinal axis of the device.

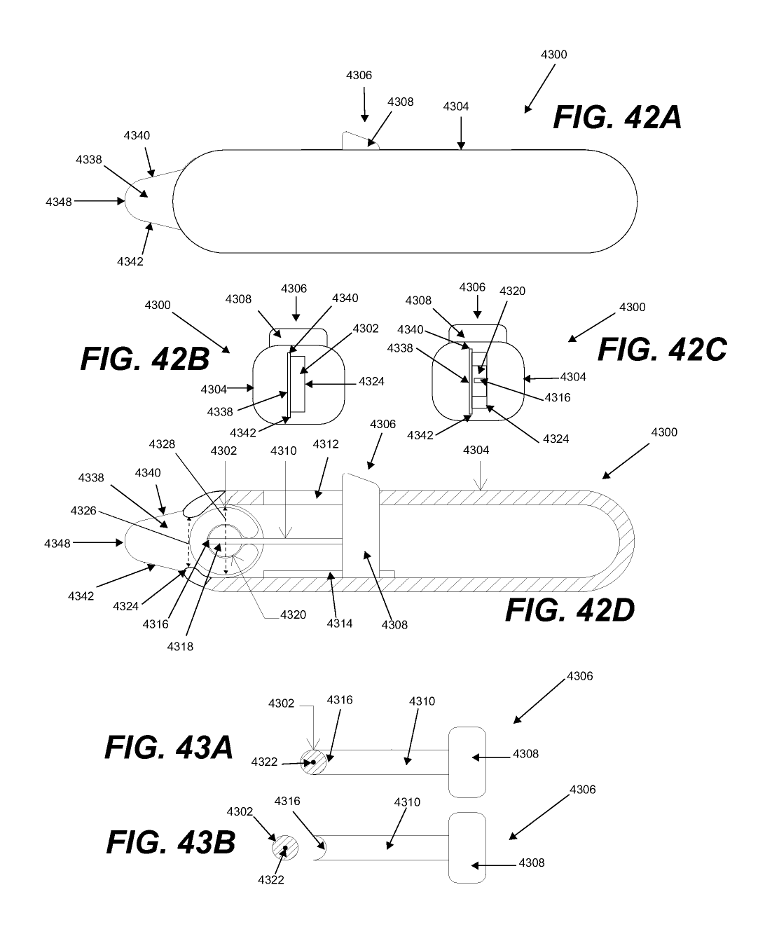

FIGS. 42A to 42D, for example, depict a delivery system 4300 for an elongate orthopedic device 4302, wherein the delivery system 4300 comprises a housing 4304 with an actuating assembly 4306 which may be used to displace the orthopedic device 4302 out of the system 4300. The actuating assembly 4306 may comprise a slidable member 4308 with a push member 4310. The movement and/or alignment of the slidable member 4308 may be limited by the opening 4312 from which the slidable member 4308 protrudes from the housing 4304, and/or by a groove or recess 4314 with which the slidable member 4308 may form a sliding interfit. In other examples, the actuating assembly may have other configurations which may include knobs or levers which rotate or pivot, for example. The actuating assembly may also optionally comprise locking interfaces or bias members (e.g. springs) to facilitate manipulation of the orthopedic device.

In this particular example, the push member 4310 comprises a linear structure with a generally rectangular axial cross-sectional shape. In other examples, the push member may comprise a non-linear configuration or a branched configuration with multiple ends. The orthopedic device 4302 in FIG. 42D is oriented in the same plane as the orientation of the slidable member 4308, but in other examples, the orthopedic device may be oriented at a different angle. The push member 4310 in FIG. 42D is configured to pass through a groove or channel 4318 of a holding member 4320, such that when the slidable member 4308 is in its retracted position, the distal end 4316 of the push member 4310 is contacting the inner curvature of the orthopedic device 4302, but in other examples, the distal end 4316 may be spaced away from surface of the orthopedic device 4302. As depicted in FIGS. 43A and 43B, the distal end 4316 of the push member 4310 may have a complementary shape to the region of the orthopedic device 4302 that it contacts, such as a concave shape that is complementary to the convex surface of the orthopedic device 4302. In other examples, the distal end of the push member and/or the orthopedic device may be configured so that the distal end may push against the core member 4322 of the orthopedic device 4302. In some instances, the orthopedic device may have an exposed core, such as the various examples in FIGS. 7A to 7C, but in other instances, the distal end of the push member may be configured to pierce through the articular layer of the orthopedic device to contact the core member.

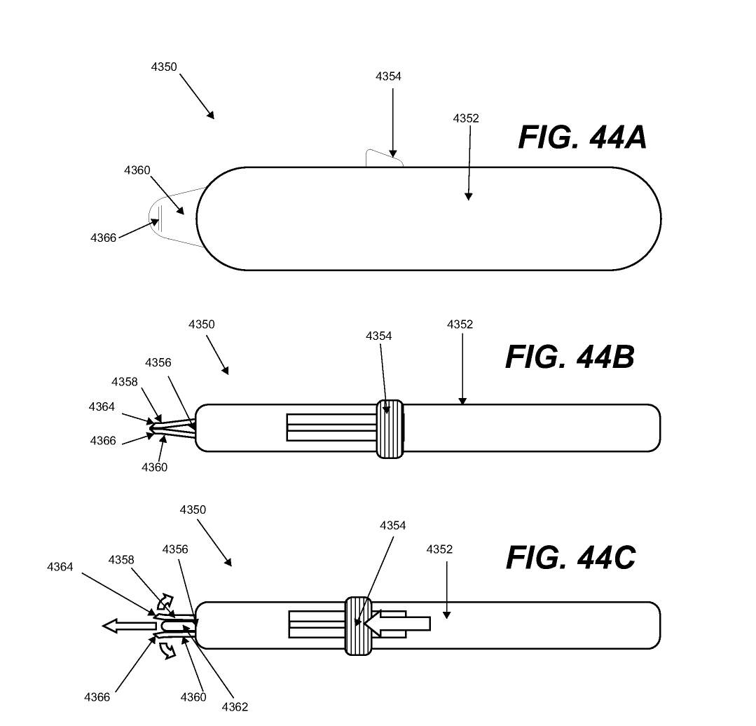

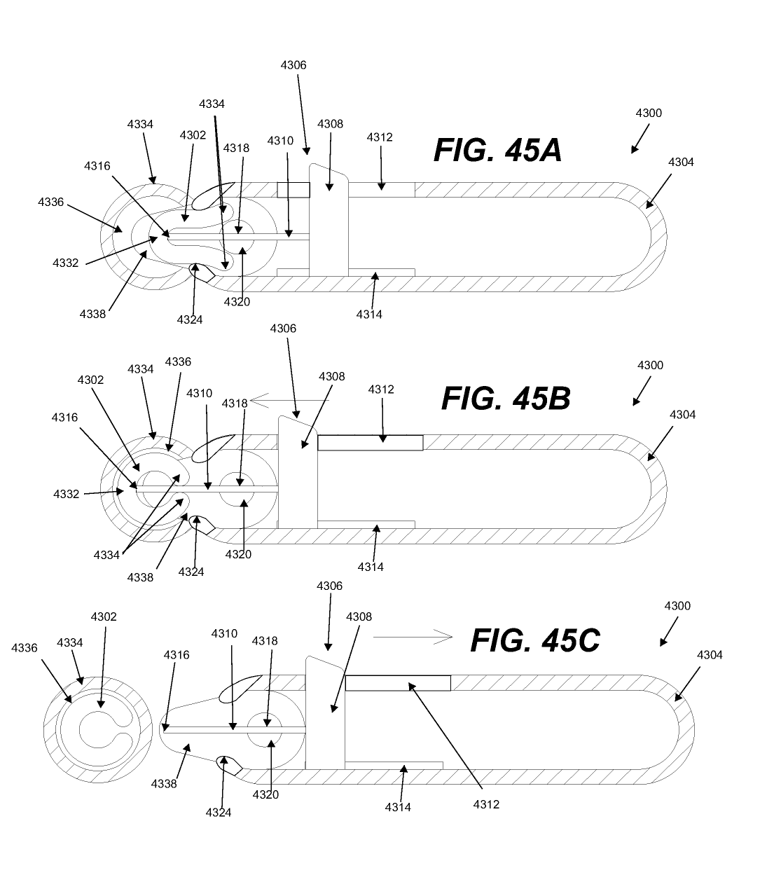

Referring back to FIG. 42D, during delivery, the orthopedic device 4302 may pass through a delivery opening 4324 which may be configured to compress or deform the orthopedic device 4302. In this example, the larger transverse dimension 4326 of the delivery opening 4324 is smaller than the larger transverse dimension 4328 of the orthopedic device 4302 along its movement pathway. As depicted in FIGS. 45A, and 45B, as the slidable member 4308 is actuated and moved from its retracted position in FIG. 44A toward its extended position in FIG. 45B, the push member 4310 exerts force on the midbody 4330 of the orthopedic device 4302 along a direction that intersects or is orthogonal to the arcuate longitudinal axis of the orthopedic device 4302. As the midbody 4330 is pushed out, the ends 4332 of the orthopedic device 4302 slide around and off the mounting member 4320 and the midbody 4330 is compressed to a smaller profile by the delivery opening 4324. The smaller profile may permit the use of a smaller incision or opening in the joint capsule 4334 to deliver the orthopedic device 4302 into the joint space 4336.

As illustrated in FIGS. 42A through 42D, the delivery system 4300 may comprise an optional tongue 4338, guide or blade about the delivery opening 4324. In some examples, the tongue 4338 may be used to position or align the delivery opening 4324 with an opening through the joint capsule 4334 or into the joint space 4336. In some instances, the tongue may also be used to maintain the orientation of the orthopedic device 4302 as it exits the delivery opening 4324. In still other examples, the tongue 4338 may be used as a retractor during an implantation procedure to support the opening in the joint capsule and/or to push apart the bony surfaces of a joint. The tongue 4338 may be rigid, semi-rigid or a flexible, and may or may not comprise an articulation with the housing 4304, such as a hinge joint. In some examples, the tongue 4338 may comprise a beveled, sharpened or cutting edge and/or tip that may be used to form and/or widen an incision or tissue opening. As illustrated, the sides 4340 and 4342 of the tongue 4338 may have a tapered configuration, but in other examples, may have a generally parallel or divergent configuration. The sides 4340 and 4342 of the tongue 4338 may also be curved or angled away from the plane of the tongue to provide additional guidance. In some examples, one or more portions of the tongue may be angled or otherwise oriented toward or away from the delivery opening, or may be generally perpendicular to the axial cross-sectional plane of the delivery opening. The distal tip 4348 of the tongue may be rounded squared or pointed, for example, and may optionally comprise a tissue engaging structure such as a barb structure. Although the delivery system 4300 depicted in FIGS. 42A to 44D comprises a delivery opening 4324 having a slot-like configuration or an opening having a first transverse dimension greater than a second transverse dimension, in other examples, the delivery opening may be circular, square or other more symmetrically shaped opening about the delivery axis.

In some examples, two or more tongue or guide structures may be provided about the delivery opening. For example, in FIGS. 44A to 44C, the delivery system 4350 comprises a housing 4352 with an actuating assembly 4354, a delivery opening 4356 and two guide structures 4358 and 4360. The two guide structures 4358 and 4360 may have the same or different configuration, and may be located symmetrically or asymmetrically with respect to the delivery opening 4356. In some examples, one or both guide structures may be configured to move or deflect, such that the guide structures comprise a first configuration with a reduced spacing or cross-sectional profile, and a second configuration with an increased spacing or cross-sectional profile. In the first configuration with the reduced spacing, the guide structures may or may not be contacting each other. In other examples, one guide structure may be movable or flexible while the other guide structure has a fixed position. The guide structures may or may not be biased to either the first configuration or the second configuration. In some examples, the spacing between the guide structures may be directly user controlled by an actuating mechanism (e.g. pull wires), but in other embodiments, the guide structures may be biased to the first configuration but are separated or spread apart when the orthopedic device passes between them. A guide structure may be biased by a spring mechanism, or may comprise a flexible material. In the particular embodiment depicted in FIGS. 44A to 44C, the guide structures 4358 and 4360 comprise a semi-rigid material and are fixed with respect to the housing 4352 and configured to contact each other until the orthopedic device 4362 contacts and pushes by the guide structures 4358 and 4360. As the orthopedic device 4362 is pushed through, the distal sections 4364 and 4366 of the guide structures 4358 and 4360 are able to flex apart, permitting passage of the orthopedic device 4362. As shown in FIGS. 44A to 44C, the distal sections 4364 and/or 4366 may be optionally angled with respect to the rest of the guide structures 4358 and 4360. In some instances, angling may further reduce the cross-sectional profile of the guide structures 4358 and 4360 to facilitate insertion of the guide structures 4358 and 4360 into narrower spaces, and may also facilitate passage of the orthopedic device 4362 providing increased distal retraction. As one or more guide structures 4358 and 4360 are separated, adjoining tissues may also be retracted or displaced away from the orthopedic device 4362.

As mentioned previously, the delivery systems described herein may be configured to implant orthopedic devices of various sizes shapes to a variety of joints. In the implantation procedure depicted in FIGS. 45A to 45C, the delivery system 4300 may be used to implant the orthopedic device 4302 into an interphalangeal joint. In this particular example, a delivery system 4300 pre-loaded with an arcuate orthopedic device 4302 is removed from its sterile packaging. The joint is palpated or otherwise identified, with or without traction or other joint manipulation (e.g. flexion, extension). The skin region about the patient's affected joint is prepped and optionally draped in the usual sterile fashion, and local, regional or general anesthesia is achieved. An anesthetic such as Marcaine, or other type of fluid such as sterilized water or a contrast agent, may be injected into the joint to provide analgesia and/or cause joint expansion. An arthrotomy incision into the joint space is made through the joint capsule 4334 to access the joint space 4336. In some embodiments, the arthrotomy incision may be performed using a stab or cut incision from a trocar or a scalpel, or by an optional delivery system comprising a tongue member with a cutting edge or incision mechanism. In some examples, the tapered shape of the tongue member enlarges at least one dimension of the incision as the tongue member is inserted through the opening or pathway into the joint space. The joint space 4336 may be optionally irrigated, and any osteophytes and/or loose cartilaginous material may be removed. In some examples, the housing of the delivery system may optionally comprise a luer connector and a tubing system in communication with the delivery opening to facilitate irrigation or removal of material. In some specific examples, a portion of the tubing system may comprise one or more lumens joined or integrated with the distal end of one or more guide structures. One or more lumens of the tubing system may also be joined or integrated with the distal end of the push member.

Referring back to FIG. 45B, once the orthopedic device 4302 has been seated in the joint space 4336, the tongue member 4338 may be withdrawn from the joint space and the incision or opening to the joint space may be closed by sutures, adhesives, or other closure procedures or devices. The slidable member 4308 and the push member 4316 may or may not be retracted back into the housing 4303 before the tongue member 4338 is withdrawn.

It will be understood that the foregoing is only illustrative of the principles of the invention, and that various modifications, alterations, and combinations can be made by those skilled in the art without departing from the scope and spirit of the invention. Any of the embodiments of the various orthopedic devices disclosed herein can include features described by any other orthopedic devices or combination of orthopedic devices herein. For example, at least the following orthopedic device as indicated by reference numbers may have features that can be combined or interchanged with other orthopedic devices. Furthermore, any of the embodiment of the various orthopedic device delivery and/or retrieval systems can be used with any of the orthopedic devices disclosed, and can include features described by any other orthopedic device delivery and/or retrieval systems or combination of orthopedic device delivery and/or retrieval systems herein. For example, at least the following orthopedic device, orthopedic device delivery and/or retrieval systems as indicated by reference numbers may have features that can be combined or interchanged with other orthopedic device delivery and/or retrieval systems. Accordingly, it is not intended that the invention be limited, except as by the appended claims. For all of the embodiments described above, the steps of the methods need not be performed sequentially.

What is claimed as new and desired to be protected by Letters Patent of the United States is:

1. A joint treatment system, comprising:
a delivery instrument comprising a housing, a housing cavity, a delivery opening in communication with the housing cavity, a slidable actuator joined to a push member, and a tongue member protruding from the housing in proximity to the delivery opening, wherein the push member has a movement path; and
an non-linear orthopedic device located in the housing cavity, the non-linear orthopedic device comprising a first end, a second end, a non-linear body therebetween, wherein the non-linear orthopedic device is oriented in the housing cavity so that the non-linear body is in closer proximity to the delivery opening than both the first and second ends.

2. The joint treatment system of claim 1, wherein the delivery instrument further comprises a mounting member located in the housing cavity between the delivery opening and at least a portion of the slidable actuator.

3. The joint treatment system of claim 2, wherein the non-linear orthopedic device is mounted on the mounting member.

4. The joint treatment system of claim 2, wherein the push member is configured to pass through at least a portion of the mounting member.

5. The joint treatment system of claim 1, wherein the non-linear orthopedic device has a transverse dimension relative to the movement path of the push member that is greater than a corresponding transverse dimension of the delivery opening.

6. The joint treatment system of claim 1, wherein the push member comprises a distal end with a complementary shape to a surface of the non-linear orthopedic device.

7. The joint treatment system of claim 1, wherein the non-linear orthopedic device has a first position and a second position, wherein the second position is closer to the delivery opening.

8. The joint treatment system of claim 1, wherein the tongue member has a tapered configuration.

9. The joint treatment system of claim 1, wherein the tongue comprises a blunt tip section having a maximum transverse dimension that is smaller than the larger transverse dimension of the orthopedic device.

10. The joint treatment system of claim 1, wherein the delivery instrument comprises at least two tongue members.

11. The joint treatment system of claim 10, wherein at least one of the tongue members is biased toward another tongue member.

12. The joint treatment system of claim 11, wherein at least two of the tongue members are biased into contact each other.

13. The joint treatment system of claim 1, wherein the tongue member comprises a cutting structure.

14. The joint treatment system of claim 10, wherein the tongue member is configured to displace away from the delivery opening by the non-linear orthopedic device.

15. The joint treatment system of claim 1, wherein the non-linear orthopedic device is an arcuate orthopedic device.

16. A joint treatment system, comprising:
a delivery instrument comprising a movable actuating assembly and a movement passage having a cross-sectional area with minimum first dimension and a second dimension transverse to the first dimension; and
an non-linear elongate orthopedic device releasably coupled to the delivery instrument, the non-linear elongate orthopedic device having a first end, a second end, and a non-linear body therebetween and a non-linear longitudinal axis between the first and second ends;
wherein the non-linear elongate orthopedic device is oriented with respect to the delivery instrument such that a portion of the non-linear longitudinal axis is transverse to the movement passage of the movable actuating assembly.

17. The joint treatment system of claim 16, wherein the delivery instrument further comprises a housing with a housing cavity and a delivery opening in communication with the housing cavity.

18. The joint treatment system of claim 17, wherein the cross-sectional area with the minimum first dimension and the second dimension transverse to the first dimension is located at the delivery opening.

19. The joint treatment system of claim 17, wherein the portion of the non-linear longitudinal axis of the orthopedic device that is transverse to the movement passage of the movable actuating assembly is located between the movable actuating assembly and the delivery opening.

20. The joint treatment system of claim 17, wherein the delivery opening is a tapered delivery opening.

21. The joint treatment system of claim 16, wherein the movable actuating assembly comprises a slidable member and a push member.

22. The joint treatment system of claim 16, wherein the delivery instrument further comprises a first guide member distal to the cross-sectional area with the minimum first dimension and the second dimension transverse to the first dimension.

23. The joint treatment system of claim 22, wherein the first guide member is a flat guide member.

24. The joint treatment system of claim 23, wherein the flat guide member is a flexible flat guide member.

25. The joint treatment system of claim 23, wherein the flat guide member comprises a tongue member with a tapered configuration and a rounded distal tip.

26. The joint treatment system of claim 16, wherein the delivery instrument further comprises a mounting member between at least a portion of the movable actuating assembly and the cross-sectional area with the minimum first dimension and the second dimension transverse to the first dimension.

27. The joint treatment system of claim 26, wherein the non-linear elongate orthopedic device is releasably mounted on the mounting member.

28. The joint treatment system of claim 26, wherein at least a portion of the movable actuating assembly movably positionable through the mounting member.

29. The joint treatment system of claim 22, wherein the delivery instrument further comprises a second guide member about the delivery opening.

30. The joint treatment system of claim 29, wherein at least one of the first and second guide members are at least partially biased toward the other guide member.

31. The joint treatment system of claim 30, wherein at least one of the first and second guide members are configured to separate from the other guide member with passage of the orthopedic device between the first and second guide members.

32. A method for treating a joint, comprising:
positioning an orthopedic device about a joint, wherein orthopedic device comprises a first end, a second end, and a non-linear body therebetween and a non-linear longitudinal axis between the first and second ends; and
pushing against the non-linear body such that at least a portion of the non-linear body enters the joint before the first and seconds ends.

33. The method of claim 32, wherein positioning the orthopedic device about the joint comprises positioning a delivery system containing the orthopedic device about the joint.

34. The method of claim 33, further comprising inserting a guide assembly of the delivery system into tissue surrounding the joint.

35. The method of claim 33, further comprising bending the guide assembly of the delivery system.

36. The method of claim 35, wherein the bending occurs while the guide assembly is at least partially located within the joint space.

37. The method of claim 35, further comprising increasing access to the joint space along at least one dimension of tissue adjacent to the joint space while inserting the guide assembly.

38. The method of claim 35, wherein bending the guide assembly comprises bending a first guide member of the guide assembly.

39. The method of claim 38, wherein bending a first guide member comprises flexing the first guide member away from a second guide member of the guide assembly.

40. The method of claim 39, further comprising bending the second guide member from the first guide member.

41. The method of claim 38, wherein bending the first guide member occurs while forcing the non-linear body against a surface of the first guide member.

42. The method of claim 32, wherein pushing against the non-linear body comprises pushing against an inner surface of the non-linear body.

43. The method of claim 34, further comprising withdrawing the guide member from the tissue surrounding the joint.
